(19) United States
(12) Patent Application Publication
(10) Pub. No.: US 2020/0369685 A1
(43) Pub. Date: Nov. 26, 2020

(54) BORONIC ESTER PRODRUGS AND USES THEREOF

(71) Applicants: Massachusetts Institute of Technology, Cambridge, MA (US); Dana-Farber Cancer Institute, Inc., Boston, MA (US)

(72) Inventors: Jeremiah A. Johnson, Boston, MA (US); Paiman Ghoroghchian, Boston, MA (US); Yivan Jiang, Revere, MA (US); Hung Vanthanh Nguyen, Cambridge, MA (US); Irene Ghobrial, Boston, MA (US); Alexandre Detappe, Boston, MA (US)

(21) Appl. No.: 16/825,269

(22) Filed: Mar. 20, 2020

Related U.S. Application Data
(60) Provisional application No. 62/850,492, filed on May 20, 2019.

(51) Int. Cl. C07F 5/02; C08L 85/04; A61K 47/59

(52) U.S. Cl. CPC C07F 5/02; A61K 47/59; C08L 85/04

(57) ABSTRACT

Disclosed herein are compounds of Formula (I) or (II). The compounds include an agent (e.g., pharmaceutical agent, cosmetic agent, or nutraceutical agent) through a linker that includes a boronic ester moiety in the backbone of the linker. The compounds may be monomers. Also provided are polymers prepared by polymerizing the monomers. The polymers may be useful for delivering the agent to a subject, tissue, biological sample, or a cell. Also provided are methods of preparing the polymers, compositions and kits comprising the polymers, and methods of use (e.g., use in delivering the agent, treating a disease, preventing a disease, diagnosing a disease) involving the polymers or compositions. The structure of the boronic ester moiety may be fine tuned so that the properties related to delivery to a subject, biological sample, tissue, or cell may be fine tuned.

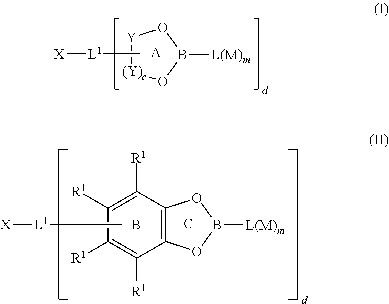

### RELATED APPLICATIONS

The present application claims priority under 35 U.S.C. § 119(e) to U.S. provisional application, U.S. Ser. No. 62/850,492, filed May 20, 2019, which is incorporated herein by reference.

### GOVERNMENT SUPPORT

This invention was made with government support under grant number RO1 CA220468 awarded by the National Institutes of Health. The government has certain rights in the invention.

### BACKGROUND OF THE INVENTION

Delivery of agents (e.g., drugs) via nanoparticles or other delivery vehicles has been previously investigated. The linker between the vehicle and pharmaceutical agent is important for successful delivery with a balance existing between stability and deliverability. Preferably, the linker is stable under physiological conditions until the vehicle has reached its target destination at which point the linker is cleaved, allowing the agent to be delivered. Linkers may also be utilized to tune potency, efficacy, bioavailability, toxicity, absorption, distribution, metabolism, excretion, tolerability, compliance, and/or a combination thereof, making linker stability and tunability key components for successful delivery.

Previously, delivery systems comprising a covalent linker have been developed for the delivery of chemotherapeutic drugs, such as ixazomib and bortezomib, via functionalization of the drugs through their boronic acid moiety. A challenge associated with these reported delivery systems is poor linker stability in aqueous solutions and/or poor linker tunability. For example MIDA (N-methyliminodiacetic acid, 1) based boronate ligands were found to have poor stability once the secondary amine was functionalized with an alkyl change longer than a methyl group (Ashley, J. C, et al., J. Med. Chem., 2014, 57, 5282). Increased steric hindrance made it more difficult for the nitrogen to coordinate with the boron, leading the ligand to be ineffective at increasing the stability of the boronate functionality in aqueous solutions (PBS, blood, etc.). An additional boronic ester linker based on 2 below was also reported, but it was also found to have poor stability in aqueous solutions due to the boronate moiety (Ashley, J. C, et al., J. Med. Chem., 2014, 57, 5282). A boronic ester of 3 was employed to make a prodrug for ixazomib, a drug used for the treatment of multiple myeloma. Linkers 1, 2, and 3 were employed with bortezomib, a multiple myeloma and mantle cell lymphoma chemotherapy, to make liposomes which have known stability issues in the blood due to dilution past the critical micellar concentration (Ashley. J. C, et al., J. Med. Chem., 2014, 57, 5282). Ultimately, these linkers were not tunable. Linker 3 was unable to be functionalized with other moieties outside of the boronic acid drug (Ashley, J. C, et al., J. Med. Chem., 2014, 57, 5282). Additionally, the boronic ester of 4, the oxaazaborolidine 5, and the diazaborolidine of 6 were previously reported as linkers for bortezomib, but the boronate functionality in each case exhibited stability issues in aqueous solutions and were limited to use with trialkoxysilyl functionality to bind to silica (Pasqua, L, et al., PCT Publication WO 2016/174693 A1, 2016). Additionally, each of these examples exhibited poor biological data and showed little improvement in comparison to free bortezomib and other boronic drugs. Ultimately, there remains a need for improved agent delivery systems.

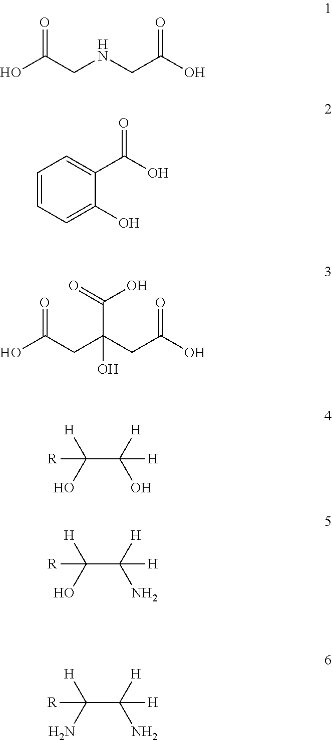

Multiple Myeloma (MM) is a fatal plasma cell dyscrasia that progresses from precursor states of monoclonal gammopathy of undetermined significance (MGUS) and smoldering multiple myeloma (SMM).^{1,2} The incidence of MGUS is about 3% of the general population aged 50 years or older.^{3,4} Proteasome inhibitors (PI) are a standard of care treatment in MM and are often used in combination with an immunomodulator (Lenalidomide, Pomalidomide), and a corticosteroid agent (Dexamethasone).^{5,6} PIs target the ubiquitin proteasome pathway (UPP) which is heavily relied upon due to the increased demand of immunoglobin production in MM. The UPP serves as an important regulator of cellular functions such as cell growth and cell survival across many cell types.^{7,8} While PIs were originally investigational probes to study the catalytic function of the proteasome, they were quickly considered as therapeutic agents for MM due to their tremendous efficacy in ablating tumor growth and inducing apoptosis.⁹ Bortezomib (Btz) soon became the first-in-class PI that was FDA-approved in 2003 for the treatment of MM, and is currently widely used as a front line treatment for MM.¹⁰ Nonetheless, while Btz has been a front-line therapy for over a decade, it still suffers from several drawbacks including high degrees of neurotoxicity, thrombocytopenia, and lymphopenia.¹¹ Btz also suffers from poor drug-like properties including poor water solubility and stability.¹¹ Altogether, these factors severely limit Btz's pharmacokinetic parameters, such as maximum serum concentration and exposure times, which impose a very narrow therapeutic index (TI) that subsequently limit its effectiveness in MM.

To overcome these pitfalls, efforts in drug discovery were aimed at establishing new PI candidates. For example, Carfilzomib (CFZ), a second-generation irreversible PI was developed and subsequently approved by the FDA in 2012.¹² Structurally distinct from its first-generation counterpart, CFZ exhibit higher stability of up to 14 days at room temperature, as well as lower toxicity due to higher target specificity.¹³ Despite slight improvements in patient outcomes in clinical trials, CFZ requires a more cumbersome treatment with long continuous infusion sessions instead of its predecessor's subcutaneous injection route. Another attempt to tackle Btz's drawbacks from a drug discovery standpoint culminated in Ixazomib (Ixa), a structural analog of Btz that was approved in 2015. In this PI, the active site is complexed to a citric acid molecule, allowing the molecule to be administrated orally. While its outperformance over Btz has not been proven in terms of efficacy, its ease of use enabled it to be quickly adopted in clinic. However, all these PIs still lack target specificity and generate undesired toxicities.^{14,15}

As an alternative to medicinal chemistry efforts, drug delivery—specifically via nanotechnology—offers another approach of alleviating Btz's drawbacks without the need of re-discovering entirely new drug candidates. Towards this goal, several attempts at loading free Btz drug or conjugated Btz prodrugs onto drug delivery systems (DDSs) have been examined, ranging from liposome formulations via encapsulation to micelle formations via self-assembly processes.¹⁶⁻²⁹ While incorporating Btz onto a DDS improves its stability and in-vivo pharmacokinetics, the final size of the Btz-loaded vehicle typically falls into the 100-200 nm range for liposomes.¹⁶⁻²² or ˜50 nm for micelles.²³⁻²⁷ This size range is not be optimal for MM therapies, as these sizes cannot effectively penetrate and accumulate in the diseased plasma cells that either reside in the bone marrow niche or are non-vascularized circulating tumor cells.³⁰ Moreover, existing issues with liposome-based DDSs such as poor tissue penetration or burst, uncontrolled payload release remain unsolved.³¹⁻³³ To address these shortcomings, various studies have aimed to exploit the boronic acid moiety on Btz to impose more controlled release and exposure profile via prodrug formation processes.²¹⁻²⁹ Most of these boronic ester prodrugs leverage naturally-occurring binding motifs including catechol/dopamine analogues,^{24,26-29} natural diols like pinanediol²³ and sugar-based^{21,25} compounds. These strategies are limited to the availability of these linker structures; this would in turn prevent this prodrug approach to be fully leveraged via rationally designing linker structures to afford a precise and, desirable in vivo release profile.

### SUMMARY OF THE INVENTION

Previous work by the group has afforded a fully covalent bottlebrush (BBP) and brush-arm star polymer (BASP) delivery platform.³⁴⁻³⁹ Constructed from macromonomers bearing precisely one payload per unit, these polymers are synthesized via ring-opening metathesis polymerization (ROMP), where the feed ratios of monomers readily translate to the final composition of the resulting polymer. Furthermore, the payload release kinetics can be rationally optimized via chemically modifying the linker that conjugate the respective payload to its macromonomer building block.⁴⁰⁻⁴¹ The size of these polymers can also be controlled via systematic variations of the polymer composition and nanoarchitecture, allowing for a wide range of selection depending on the target diseased tissues.³⁷⁻⁴³ This extensive toolbox has thus far allowed us to incorporate a variety of therapeutic agents (or combinations thereof) followed by their subsequent in vivo delivery in solid mouse cancer models.³⁴⁻⁴³

Herein, the attempt to deploy this platform towards MM, a hematological malignancy with liquid tumors that are challenging to target and thus, necessitates further specializations of this unique BBP nanoarchitecture is reported. To achieve this goal, Btz was conjugated onto the macromonomer scaffold via a fully synthetic linker, affording the freedom to tinker with its structure to influence the final release profile. Subsequent ROMP of these monomers afforded polymers that were designed to be ˜10 nm in size, which lies the same regime as therapeutic proteins and is hypothesized to facilitate efficient accumulation and penetration in MM's aforementioned tumor targets.^{30,44} In vitro cell assays revealed Btz-BBP's comparable toxicity compared to its free Btz counterpart, confirming the ability of Btz-BBP to release its payload in the tumor microenvironment. Furthermore, in vivo toxicity of Btz-BBP in healthy BALB/c mice showed a marked enhancement of ≥20-fold maximum tolerated dose (MTD) compared to free Btz, which in turn afforded improvement in efficacy in tumor-bearing mice models. In a subcutaneous model, Btz-BBP outperforms its parent drug, both at the same dose (Btz's MTD), and further improves at Btz-BBP's MTD. Furthermore, in an aggressive orthotopic model where the parent Btz yielded no improvements in treatment outcome. Btz-BBP was able to effectively suppress tumor growth and extend the survival rate in both subcutaneous and orthotopic mouse models of Multiple Myeloma by 2-fold and 3-fold, respectively. Altogether, these results unequivocally validated the DDS design principles and demonstrated an improved TI of this promising PI.

Described herein are compounds that include an agent through a linker that includes a boronic ester moiety in the backbone of the linker. In certain embodiments, the agent is a pharmaceutical agent (e.g., bortezomib), cosmetic agent, or nutraceutical agent. In certain embodiments, the compounds include a polymerization handle and are thus monomers. Also provided are polymers (e.g., homopolymers, copolymers, charged polymers, hydrophilic polymers, linear polymers, branched polymers, brush polymers, bottlebrush polymers) prepared by polymerizing the monomers (e.g., via ring-opening metathesis polymerization, condensation polymerization, addition polymerization, radical polymerization, cationic polymerization, anioinc polymerization). The polymers may be useful for delivering the agent to a subject, tissue, biological sample, or a cell. Also provided are methods of preparing the polymers, compositions and kits comprising the polymers, and methods of use (e.g., use in delivering the agent, treating a disease, preventing a disease, diagnosing a disease) involving the polymers or compositions.

The structure of the boronic ester moiety may be fine tuned so that the properties related to delivery (e.g., stability under physiological conditions) to a subject, biological sample, tissue, or cell may be fine tuned. The polymers with desired properties related to delivery may result in higher potency, higher efficacy, higher bioavailability, less toxicity, higher absorption, larger distribution, faster or slower metabolism, faster or slower excretion, wider therapeutic window of the agent, higher tolerability, higher compliance of the subject, and/or a combination thereof. Without being bound by any particular theory, the bulk of the boronic ester moiety may be fine tuned so that the properties related to delivery may be fine tuned.

In one aspect, a compound of the disclosure is of the Formula (I) or (II):

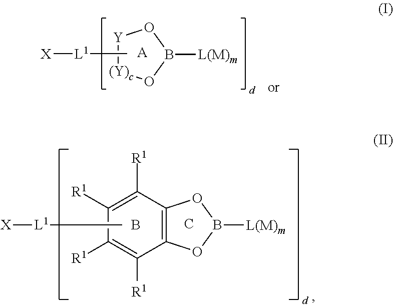

or a salt thereof, wherein X, L¹, Y, c, L, M, m, and dare as described herein.

In certain embodiments, a compound of Formula (I) is of Formula (I-1):

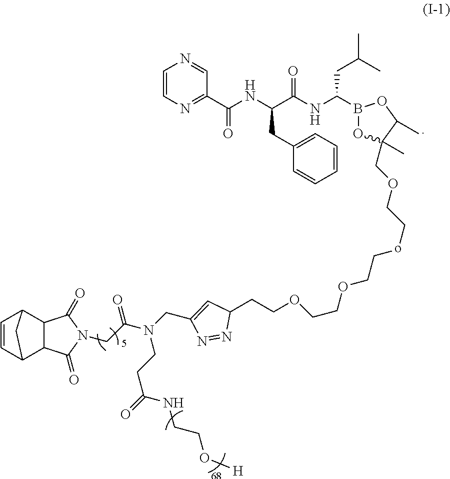

or a salt thereof.

In another aspect, the present disclosure provides methods of preparing polymers comprising polymerizing a monomer described herein. In certain embodiments, the step of polymerizing comprises the presence of a metathesis catalyst.

In another aspect, the present disclosure provides polymers prepared by a method described herein.

In another aspect, the present disclosure provides compositions comprising a polymer described herein and optionally an excipient.

In another aspect, the present disclosure provides kits comprising a polymer or a composition described herein; and instructions for using the polymer or composition.

In another aspect, the present disclosure provides methods of delivering an agent to a biological sample, tissue, or cell, the methods comprising contacting the biological sample, tissue, or cell with a polymer described herein.

In another aspect, the present disclosure provides methods of delivering an agent to a subject in need thereof, the methods comprising administering to the subject in need thereof a polymer described herein.

In another aspect, the present disclosure provides methods of treating a disease in a subject in need thereof, the methods comprising administering to the subject in need thereof a therapeutically effective amount of a polymer or composition described herein, wherein at least one instance of M is a therapeutic agent.

In another aspect, the present disclosure provides methods of preventing a disease in a subject in need thereof, the methods comprising administering to the subject in need thereof a therapeutically effective amount of a polymer or composition described herein, wherein at least one instance of M is a prophylactic agent.

In another aspect, the present disclosure provides methods of diagnosing a disease in a subject in need thereof, the methods comprising administering to the subject in need thereof a therapeutically effective amount of a polymer or composition described herein, wherein at least one instance of M is a diagnostic agent.

In another aspect, the present disclosure provides uses (e.g., uses in the methods described herein) of the polymers, compositions, and kits described herein.

The details of certain embodiments of the invention are set forth in the Detailed Description of Certain Embodiments, as described below. Other features, objects, and advantages of the invention will be apparent from the Definitions. Figures. Examples, and Claims. It should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

FIG. 1 shows the synthesis of Btz-BBP. Bortezomib (Btz) is modified to incorporate a linker containing an azide to form the compound Btz-N₃. Btz-N₃ is then modified through Click chemistry to generate the monomer Btz-MM. Btz-MM is used as a monomer in ring-opening metathesis polymerization (ROMP) to generate the bottlebrush polymer (BBP) Btz-BBP.

FIG. 2 shows a mass spectrum using MALDI-TOF of Btz-BBP.

FIG. 3 shows a GPC graph of Btz-BBP.

FIG. 4 shows a mass spectrum using MALDI of Ixa-BBP.

FIG. 5 shows a GPC graph of Ixa-BBP.

FIG. 6 shows a mass spectrum using MALDI of BtzC-BBP.

FIG. 7 shows a GPC graph of BtzC-BBP.

FIG. 8A shows the in vitro cell viability in MM1S.

FIG. 8B shows in vitro cell viability in KMS11.

FIGS. 9A to 9E show in vivo maximum tolerated dose by survivability wherein each group (n=3-5) was dosed 4 times besides the 20 mg/kg group which was dosed 3 times.

FIG. 9A shows Kaplan-Meier survival curves (n=5) of injected mice.

FIG. 9B shows body weight measurements (n=5) of intravenously injected mice with Btz-BBP at various doses. The dashed line corresponds to the 20% body weight loss toxicity threshold.

FIG. 9C shows metabolic profile of healthy BALB/c mice (n=3) that were treated for 2 weeks (2 injections/week) followed by 2 weeks of rest period (no injections) prior to blood draw and analysis.

FIG. 9D shows complete blood counts of healthy BALB/c mice (n=3) that were treated for 2 weeks (2 injections/week) followed by 2 weeks of rest period (no injections) prior to blood draw and analysis.

FIG. 9E shows white blood cell differential counts of healthy BALB/c mice (n=3) that were treated for 2 weeks (2 injections/week) followed by 2 weeks of rest period (no injections) prior to blood draw and analysis.

FIG. 10A shows evaluation of tumor accumulation and penetration of Btz-BBP (Cy5.5 labelled) by fluorescence microscopy; the tumor was harvested 1 h post-injection (iv). For efficacy evaluation, KMS11 subcutaneous tumor-bearing mice (Btz-resistant) were injected with PBS, Btz, or Btz-BBP (2 injections/week, 4 weeks), starting when largest axis of the tumor reached 5 mm length.

FIG. 10B shows the fold-change individual spider plot of tumor size progressions over the course of the study (n=5). Statistical analysis was performed using a two-tailed t-test between Btz and Btz-BBP groups.

FIG. 10C shows the fold-change tumor volume in SCID KMS11 subcutaneous mouse model wherein the dosing schedule consisted of 1 injection per week. Statistical analysis was performed using a two-tailed t-test between Btz and Btz-BBP groups.

FIG. 10D shows the Kaplan-Meier survival curves in SCID KMS11 subcutaneous mouse model wherein the dosing schedule consisted of 1 injection per week. These reveal enhancements in therapeutic outcomes of Btz-BBP over free Btz, both at the same dose and high dose. Statistical analysis was performed using a Log-Rank test. (*P<0.05, **P<0.01, ***P<0.001).

FIG. 11A shows the fold-change tumor volume in SCID MM.1A subcutaneous mouse model wherein the dosing schedule consisted of 1 injection per week.

FIG. 11B shows the percent survival in SCID MM.1A subcutaneous mouse model wherein the dosing schedule consisted of 1 injection per week.

FIG. 11C shows the individual spider plot of tumor size progressions over the course of the study (n=5). Statistical analysis was performed using a two-tailed t-test between Btz and Btz-BBP groups.

FIG. 12A shows tumors imaged via bioluminescence at day 20 for the control group which did not receive Btz.

FIG. 12B shows tumors imaged via bioluminescence at day 20 for the group which received 0.75 mg/kg of Btz per injection.

FIG. 12C shows tumors imaged via bioluminescence at day 20 for the group which received 18.75 mg/kg of Btz-BBP per injection.

FIG. 12D shows tumors imaged via bioluminescence imaging of MM.1S GFP+/LUC+ disseminated cells at day 0 for the control group which did not receive Btz.

FIG. 12E shows tumors imaged via bioluminescence imaging of MM.1S GFP+/LUC+ disseminated cells at day 0 for the group which received 0.75 mg/kg of Btz per injection.

FIG. 12F shows tumors imaged via bioluminescence imaging of MM.1S GFP+/LUC+ disseminated cells at day 0 for the group which received 18.75 mg/kg of Btz-BBP per injection.

FIG. 13 shows hydrodynamic diameter (Dh) of Btz-BBP as determined by dynamic light scattering (DLS).

FIG. 14 shows body weight monitor (BWM) during the efficacy study of a MM subcutaneous mouse model.

FIG. 15 shows BWM during the efficacy study of a MM orthotopic mouse model.

### DEFINITIONS

For convenience, certain terms employed herein, in the specification, examples and appended claims are collected herein.

Unless otherwise required by context, singular terms shall include pluralities, and plural terms shall include the singular.

The following definitions are more general terms used throughout the present application:

The singular terms “a,” “an” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2% or 1% of a given value or range of values.

Definitions of specific functional groups and chemical terms are described in more detail below. The chemical elements are identified in accordance with the Periodic Table of the Elements. CAS version. Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry. Thomas Sorrell. University Science Books. Sausalito, 1999; Smith and March March's Advanced Organic Chemistry, 5^{th} Edition. John Wiley & Sons. Inc., New York, 2001; Larock. Comprehensive Organic Transformations, VCH Publishers, Inc., New York, 1989; and Carruthers, Some Modern Methods of Organic Synthesis, 3^{rd} Edition. Cambridge University Press, Cambridge, 1987.

Compounds described herein can include one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al. Enantiomers, Racemates and Resolutions (Wiley Interscience, New York, 1981); Wilen et al., Tetrahedron 33:2725 (1977): Eliel, E. L. Stereochemistry of Carbon Compounds (McGraw-Hill, NY, 1962); and Wilen, S. H. Tables of Resolving Agents and Optical Resolutions p, 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The term “alkyl” refers to a radical of a straight-chain or branched saturated hydrocarbon group. In some embodiments, an alkyl group has 1 to 1000 carbon atoms (“C₁-C₁₀₀₀ alkyl”), 1 to 900 carbon atoms (“C₁-C₉₀₀ alkyl”), 1 to 800 carbon atoms (“C₁-C₈₀₀ alkyl”), 1 to 700 carbon atoms (“C₁-C₇₀₀ alkyl”), 1 to 600 carbon atoms (“C₁-C₆₀₀ alkyl”), 1 to 500 carbon atoms (“C₁-C₅₀₀ alkyl”), 1 to 400 carbon atoms (“C₁-C₄₀₀ alkyl”), 1 to 300 carbon atoms (“C₁-C₃₀₀ alkyl”), 1 to 200 carbon atoms (“C₁-C₂₀₀ alkyl”), 1 to 100 carbon atom (“C₁-C₁₀₀ alkyl”). In some embodiments, an alkyl group has 1 to 10 carbon atoms (“C₁-C₁₀ alkyl”), 1 to 9 carbon atoms (“C₁-C₉ alkyl”), 1 to 8 carbon atoms (“C₁-C₈ alkyl”), 1 to 7 carbon atoms (“C₁-C₇ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), 1 to 2 carbon atoms (“C₁-C₂ alkyl”), or 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents.

The term “alkenyl” refers to a radical of a straight-chain or branched hydrocarbon group having from 2 to 1000 carbon atoms and one or more carbon-carbon double bonds (e.g., 1, 2, 3, or 4 double bonds). In some embodiments, an alkenyl group has 2 to 1000 carbon atoms (“C₂-C₁₀₀₀ alkenyl”), 2 to 900 carbon atoms (“C₂-C₉₀₀ alkenyl”), 2 to 800 carbon atoms (“C₂-C₈₀₀ alkenyl”), 2 to 700 carbon atoms (“C₂-C₇₀₀ alkenyl”), 2 to 600 carbon atoms (“C₂-C₆₀₀ alkenyl”) 2 to 500 carbon atoms (“C₂-C₅₀₀ alkenyl”), 2 to 400 carbon atoms (“C₂-C₄₀₀ alkenyl”), 2 to 300 carbon atoms (“C₂-C₃₀₀ alkenyl”), 2 to 200 carbon atoms (“C₂-C₂₀₀ alkenyl”), 2 to 100 carbon atom (“C₂-C₁₀₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified (e.g., —CH═CHCH₃,

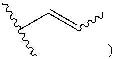

may be in the (E)- or (Z)-configuration.

The term “alkynyl” refers to a radical of a straight-chain or branched hydrocarbon group having from 2 to 1000 carbon atoms and one or more carbon-carbon triple bonds (e.g., 1, 2, 3, or 4 triple bonds). In some embodiments, an alkynyl group has 2 to 1000 carbon atoms (“C₂-C₁₀₀₀ alkynyl”), 2 to 900 carbon atoms (“C₂-C₉₀₀ alkynyl”), 2 to 800 carbon atoms (“C₂-C₈₀₀ alkynyl”), 2 to 700 carbon atoms (“C₂-C₇₀₀ alkynyl”), 2 to 600 carbon atoms (“C₂-C₆₀₀ alkynyl”), 2 to 500 carbon atoms (“C₂-C₅₀₀ alkynyl”), 2 to 400 carbon atoms (“C₂-C₄₀₀ alkynyl”), 2 to 300 carbon atoms (“C₂-C₃₀₀ alkynyl”), 2 to 200 carbon atoms (“C₂-C₂₀₀ alkynyl”), 2 to 100 carbon atom (“C₂-C₁₀₀ alkynyl”). In some embodiments, an alkynyl group has 2 to 9 carbon atoms (“C₂₋₉ alkynyl”), 2 to 8 carbon atoms (“C₂₋₈ alkynyl”), 2 to 7 carbon atoms (“C₂₋₇ alkynyl”), 2 to 6 carbon atoms (“C₂₋₆ alkynyl”), 2 to 5 carbon atoms (“C₂₋₅ alkynyl”), 2 to 4 carbon atoms (“C₂₋₄ alkynyl”), 2 to 3 carbon atoms (“C₂₋₃ alkynyl”), or 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Unless otherwise specified, each instance of an alkynyl group is independently unsubstituted (an “unsubstituted alkynyl”) or substituted (a “substituted alkynyl”) with one or more substituents.

The term “heteroalkyl” refers to an alkyl group which further includes at least one heteroatom (e.g., 1, 2, 3, or 4 heteroatoms) selected from oxygen, nitrogen, phosphorus, or sulfur within (i.e., inserted between adjacent carbon atoms of) and/or placed at one or more terminal position(s) of the parent chain. In certain embodiments, a heteroalkyl group refers to a saturated group having from 1 to 1000 carbon atoms and 1 or more heteroatoms within the parent chain (“C₁-C₁₀₀₀ heteroalkyl”), 1 to 900 carbon atoms and 1 or more heteroatoms within the parent chain (“C₁-C₉₀₀ heteroalkyl”), 1 to 800 carbon atoms and 1 or more heteroatoms within the parent chain (“C₁-C₈₀₀ heteroalkyl”), 1 to 700 carbon atoms and 1 or more heteroatoms within the parent chain (“C₁-C₇₀₀ heteroalkyl”), 1 to 600 carbon atoms and 1 or more heteroatoms within the parent chain (“C₁-C₆₀₀ heteroalkyl”), 1 to 500 carbon atoms and 1 or more heteroatoms within the parent chain (“C₁-C₅₀₀ heteroalkyl”), 1 to 400 carbon atoms and 1 or more heteroatoms within the parent chain (“C₁-C₄₀₀ heteroalkyl”), 1 to 300 carbon atoms and 1 or more heteroatoms within the parent chain (“C₁-C₃₀₀ heteroalkyl”), 1 to 200 carbon atoms and 1 or more heteroatoms within the parent chain (“C₁-C₂₀₀ heteroalkyl”), or 1 to 100 carbon atoms and 1 or more heteroatoms within the parent chain (“C₁-C₁₀₀ heteroalkyl”). In certain embodiments, a heteroalkyl group refers to a saturated group having from 1 to 10 carbon atoms and 1 or more heteroatoms within the parent chain (“C₁-C₁₀ heteroalkyl”), 1 to 9 carbon atoms and 1 or more heteroatoms within the parent chain (“C₁-C₉ heteroalkyl”), 1 to 8 carbon atoms and 1 or more heteroatoms within the parent chain (“C₁-C₈ heteroalkyl”), 1 to 7 carbon atoms and 1 or more heteroatoms within the parent chain (“C₁-C₇ heteroalkyl”), 1 to 6 carbon atoms and 1 or more heteroatoms within the parent chain (“C₁-C₆ heteroalkyl”), 1 to 5 carbon atoms and 1 or more heteroatoms within the parent chain (“C₁-C₅ heteroalkyl”), 1 to 4 carbon atoms and 1 or more heteroatoms within the parent chain (“C₁-C₄ heteroalkyl”), 1 to 3 carbon atoms and 1 or more heteroatoms within the parent chain (“C₁-C₃ heteroalkyl”), 1 to 2 carbon atoms and 1 heteroatom within the parent chain (“C₁-C₂ heteroalkyl”), or 1 carbon atom and 1 heteroatom (“C₁ heteroalkyl”). Unless otherwise specified, each instance of a heteroalkyl group is independently unsubstituted (an “unsubstituted heteroalkyl”) or substituted (a “substituted heteroalkyl”) with one or more substituents.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a saturated group having from 1 to 1000 carbon atoms and 1 or more heteroatoms within the parent chain (“heteroC₁-C₁₀₀₀ alkenyl”), 1 to 900 carbon atoms and 1 or more heteroatoms within the parent chain (“heteroC₁-C₉₀₀ alkenyl”), 1 to 800 carbon atoms and 1 or more heteroatoms within the parent chain (“heteroC₁-C₈₀₀ alkenyl”), 1 to 700 carbon atoms and 1 or more heteroatoms within the parent chain (“heteroC₁-C₇₀₀ alkenyl”), 1 to 600 carbon atoms and 1 or more heteroatoms within the parent chain (“heteroC₁-C₆₀₀ alkenyl”), 1 to 500 carbon atoms and 1 or more heteroatoms within the parent chain (“heteroC₁-C₅₀₀ alkenyl”), 1 to 400 carbon atoms and 1 or more heteroatoms within the parent chain (“heteroC₁-C₄₀₀ alkenyl”), 1 to 300 carbon atoms and 1 or more heteroatoms within the parent chain (“heteroC₁-C₃₀₀ alkenyl”), 1 to 200 carbon atoms and 1 or more heteroatoms within the parent chain (“heteroC₁-C₂₀₀ alkenyl”), or 1 to 100 carbon atoms and 1 or more heteroatoms within the parent chain (“heteroC₁-C₁₀₀ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a saturated group having from 1 to 1000 carbon atoms and 1 or more heteroatoms within the parent chain (“heteroC₁-C₁₀₀₀ alkynyl”), 1 to 900 carbon atoms and 1 or more heteroatoms within the parent chain (“heteroC₁-C₉₀₀ alkynyl”), 1 to 800 carbon atoms and 1 or more heteroatoms within the parent chain (“heteroC₁-C₈₀₀ alkynyl”), 1 to 700 carbon atoms and 1 or more heteroatoms within the parent chain (“heteroC₁-C₇₀₀ alkynyl), 1 to 600 carbon atoms and 1 or more heteroatoms within the parent chain (“heteroC₁-C₆₀₀ alkynyl”), 1 to 500 carbon atoms and 1 or more heteroatoms within the parent chain (“heteroC₁-C₅₀₀ alkynyl”), 1 to 400 carbon atoms and 1 or more heteroatoms within the parent chain (“heteroC₁-C₄₀₀ alkynyl”), 1 to 300 carbon atoms and 1 or more heteroatoms within the parent chain (“heteroC₁-C₃₀₀ alkynyl”), 1 to 200 carbon atoms and 1 or more heteroatoms within the parent chain (“heteroC₁-C₂₀₀ alkynyl”), or 1 to 100 carbon atoms and 1 or more heteroatoms within the parent chain (“heteroC₁-C₁₀₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” or “cycloalkyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”), 3 to 7 ring carbon atoms (“C₃₋₇ carbocyclyl”), 3 to 6 ring carbon atoms (“C₃₋₆ carbocyclyl”), 4 to 6 ring carbon atoms (“C₄₋₆ carbocyclyl”), 5 to 6 ring carbon atoms (“C₅₋₆ carbocyclyl”), or 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₅ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, phosphorus, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents.

In some embodiments, a heterocyclyl group is a 5-10 membered non-aromatic ring system having ring carbon atoms and 1-4 ring heteroatoms, wherein each heteroatom is independently selected from nitrogen, oxygen, phosphorus, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, phosphorus, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, phosphorus, and sulfur (“5-6 membered heterocyclyl”). In some embodiments, the 5-6 membered heterocyclyl has 1-3 ring heteroatoms selected from nitrogen, oxygen, phosphorus, and sulfur. In some embodiments, the 5-6 membered heterocyclyl has 1-2 ring heteroatoms selected from nitrogen, oxygen, phosphorus, and sulfur. In some embodiments, the 5-6 membered heterocyclyl has 1 ring heteroatom selected from nitrogen, oxygen, phosphorus, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl, and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group be monovalent or may have more than one point of attachment to another moiety (e.g., it may be divalent, trivalent, etc), although the valency may be specified directly in the name of the group. For example, “triazoldiyl” and “triazolylene” refer to a divalent triazolyl moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As understood from the above, alkyl, alkenyl, alkynyl, carbocyclyl, aryl, and heteroaryl groups are, in certain embodiments, optionally substituted. Optionally substituted refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Affixing the suffix “ene” to a group indicates the group is a polyvalent (e.g., bivalent, trivalent, tetravalent, or pentavalent) moiety. In certain embodiments, affixing the suffix “ene” to a group indicates the group is a bivalent moiety (e.g., carbocyclene refers to a carbocyclic ring which is bivalent (e.g., C₆ alkyl-carbocyclyl-C₆ alkyl)).

Exemplary carbon atom substituents include, but are not limited to, halogen, —CN, —NO₂, —N₃, —SO₂H, —SO₃H, —OH, —OR^{aa}, —ON(R^{bb})₂—N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂.═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{cc}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{d} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OC₂R^{ee}, —C(═O)N(R^{ee})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OS₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R⁹ is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —C₂H, —C₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁-6 alkyl)₂, —OP(═O)(C₁₋₆ alkyl)₂, —OP(═O)(OC₁₋₆ alkyl)₂. C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; and

each instance of X⁻ is a counterion.

In certain embodiments, the carbon atom substituents are independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted C₁₋₆ alkyl, —OR^{aa}, —SR^{aa}, —N(R^{bb})₂, —CN, —SCN, or —NO₂.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{cc}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative. N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylidencamine, N-p-methoxybenzylideneamine, N-diphenylmethyleneamine, N-[(2-pyridyl)mesityl]methyleneamine, N—(N′,N′-dimethylaminomethylene)amine. N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{cc}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl. (phenyldimethylsilyl)methoxymethyl (SMOM), benzyloxymethyl (BOM), p-methoxybenzyloxymethyl (PMBM). (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{cc})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis. T. W. Greene and P. G. M. Wuts, 3^{rd} edition, John Wiley & Sons, 1999, incorporated herein by reference.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “thiol” or “thio” refers to the group —SH.

The term “amine” or “amino” refers to the group —NH— or —NH₂.

As used herein, the term “polyethylene glycol” or “PEG” refers to an ethylene glycol polymer that contains about 20 to about 2,000,000 linked monomers, typically about 50-1,000 linked monomers, usually about 100-300. Polyethylene glycols include ethylene glycol polymer containing various numbers of linked monomers, e.g., PEG20, PEG30, PEG40, PEG60, PEG80, PEG100, PEG115, PEG200, PEG300, PEG400, PEG500, PEG600, PEG1000, PEG1500, PEG2000, PEG3350, PEG4000, PEG4600, PEG5000, PEG6000, PEG8000, PEG11000, PEG12000, PEG2000000, and any mixtures thereof.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example. Berge et al, describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

“Click chemistry” refers to a chemical approach to conjugation introduced by Sharpless in 2001 and describes chemistry tailored to generate substances quickly and reliably by joining units together. See, e.g., Kolb, Finn and Sharpless Angewandte Chemie International Edition 200140, 2004-2021; Evans. Australian Journal of Chemistry 2007 60, 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition reactions); and Diels-Alder reactions (e.g., tetrazine [4+2] cycloaddition). Examples of click chemistry reactions and click-chemistry handles can be found in, e.g., Kolb. H. C.; Finn. M. G, and Sharpless, K. B. Angew. Chem. Int. Ed. 2001, 40, 2004-2021. Kolb. H. C, and Sharless, K. B. Drug Disc. Today, 2003, 8, 112-1137; Rostovtsev, V. V.; Green L. G.; Fokin, V. V, and Shrapless, K. B. Angew. Chem. Int. Ed. 2002, 41, 2596-2599; Tomoe, C. W.; Christensen, C, and Meldal, M. J. Org. Chem, 2002, 67, 3057-3064. Wang, Q, et al., J. Am. Chem. Soc. 2003, 125, 3192-3193; Lee. L. V, et al., J. Am. Chem. Soc. 2003 125, 9588-9589; Lewis. W. G, et al., Angew. Chem. Int. Ed. 2002, 41, 1053-41057; Manetsch, R, et al., J. Am. Chem. Soc. 2004, 126, 12809-12818; Mocharla, V. P, et al., Angew. Chem., Int. Ed. 2005, 44, 116-120.

Any methods known in the art of bioconjugation can be used (e.g., click chemistry reactions). For example, the nanoparticle may comprise a click chemistry handle on its outer shell, which can react with a click chemistry handle on a targeting agent, thereby covalently linking the nanoparticle with the targeting agent. In certain embodiments, the one or more nanoparticles are conjugated to the targeting agent via click chemistry, and therefore the linker comprises a moiety derived from a click chemistry reaction (e.g., triazole, diazole, diazine, sulfide bond, maleimide ring, succinimide ring, ester, amide).

The term “average molecular weight” may encompass the number average molecular weight (M), weight average molecular weight (M_{w}), higher average molecular weight (M_{z} or M_{z}+1), GPC/SEC (gel permeation chromatography/size-exclusion chromatography)-determined average molecular weight (Mₚ), and viscosity average molecular weight (Mᵥ).

The term “average hydrodynamic diameter” (D_{H}) as used herein refers to the average size of a conjugate or particle. The average hydrodynamic diameter may or may not encompass the solvation layers of conjugate or particle, and may be determined through a number of methods including dynamic light scattering, electron microscopy (e.g., scanning electron microscopy, transmission electron microscopy), atomic force microscopy, and X-ray diffraction. The hydrodynamic diameter measured by dynamic light scattering (DLS) is defined as “the size of a hypothetical hard sphere that diffuses in the same fashion as that of the particle being measured”. In practice though, particles or macromolecules in solution are non-spherical, dynamic (tumbling), and solvated. Because of this, the diameter calculated from the diffusional properties of the particle will be indicative of the apparent size of the dynamic hydrated/solvated particle. Hence the terminology, Hydrodynamic diameter. The hydrodynamic diameter, or Stokes diameter, therefore is that of a sphere that has the same translational diffusion coefficient as the particle being measured, assuming a hydration layer surrounding the particle or molecule. The measured data in a dynamic light scattering (DLS) experiment is the correlation curve which should be a smooth, single exponential decay function for a mono-size particle dispersion (Chu. B., Annual Review of Physical Chemistry, 1970, 21, 145-174). Embodied within the correlation curve is all of the information regarding the diffusion of particles within the sample being measured. By fitting the correlation curve to an exponential function, the diffusion coefficient (D) can be calculated (D is proportional to the lifetime of the exponential decay). With the diffusion coefficient (D) now known, the hydrodynamic diameter can be calculated by using a variation of the Stokes-Einstein equation. For a polydisperse sample this curve is a sum of exponential decays.

The term “average polydispersity” (PDI) as used herein refers to a measure of the distribution of molecular size in a mixture, e.g., as determined by a chromatographic method, such as gel permeation chromatography or size exclusion chromatography, or through dynamic light scattering. Polydispersity (PDI) is a measure of the distribution of molecular mass in a given polymer. Polydispersity is calculated by: PDI=M_{w}/Mₙ (Stepto, R. F. T., et al., Pure Appl. Chem., 2009, 81.351-353). Mₙ is more sensitive to molecules of low molecular mass, while M, is more sensitive to molecules of high molecular mass. The dispersity indicates the distribution of individual molecular masses in a bath of polymers. D has a value equal to or greater than 1.

As used herein, the term “agent” means a molecule, group of molecules, complex or substance administered to an organism for diagnostic, therapeutic, preventative medical, or veterinary purposes. In certain embodiments, the agent is a pharmaceutical agent (e.g., a therapeutic agent, a diagnostic agent, or a prophylactic agent). In certain embodiments, the compounds, conjugates, or particles disclosed herein comprise an agent(s), e.g., a first therapeutic agent (e.g., at least one (including, e.g., at least two, at least three). In some embodiments, the BASP-compositions (e.g., compounds, conjugates, or particles) can further comprise a second therapeutic agent, a targeting moiety, a diagnostic moiety, e.g., as described herein. The agent(s) can be coupled to the conjugate or particle. In other embodiments, the agent(s) can be associated with a conjugate or particle. In some embodiments, a first agent can be coupled to the conjugate or particle, and a second agent, targeting moiety, and/or diagnostic moiety can be non-covalently associated with the conjugate or particle. Any of the agents disclosed herein can be used in the compounds, conjugates, particles and other compositions and methods disclosed herein.

As used herein, the term “therapeutic agent” includes an agent that is capable of providing a local or systemic biological, physiological, or therapeutic effect in the biological system to which it is applied. For example, a therapeutic agent can act to control tumor growth, control infection or inflammation, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism.

An agent, e.g., a therapeutic agent, can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof.

In some embodiments, the agent is in the form of a prodrug. The term “prodrug” refer to a compound that becomes active, e.g., by solvolysis, reduction, oxidation, or under physiological conditions, to provide a pharmaceutically active compound, e.g., in vivo. A prodrug can include a derivative of a pharmaceutically active compound, such as, for example, to form an ester by reaction of the acid, or acid anhydride, or mixed anhydrides moieties of the prodrug moiety with the hydroxyl moiety of the pharmaceutical active compound, or to form an amide prepared by the acid, or acid anhydride, or mixed anhydrides moieties of the prodrug moiety with a substituted or unsubstituted amine of the pharmaceutically active compound. Simple aliphatic or aromatic esters, amides, and anhydrides derived from acidic groups may comprise prodrugs. In some embodiments, the conjugate or particle described herein incorporates one therapeutic agent or prodrug thereof. In some embodiments, the conjugate or particle described herein incorporates more than one therapeutic agents or prodrugs.

In some embodiments, the agent, e.g., a therapeutic agent, a small molecule. As used herein, the term “small molecule” can refer to compounds that are “natural product-like.” However, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary agents, e.g., a therapeutic agents, in the BBP-compositions include, but are not limited to, those found in Harrison's Principles of Internal Medicine, 13th Edition, Eds. T. R. Harrison et al., McGraw-Hill N.Y., NY; Physicians' Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

In some embodiments, exemplary therapeutic agents in the BBP-compositions include, but are not limited to, one or more of the agents listed in Paragraph [0148] of U.S. Pat. No. 9,381,253, incorporated by reference herein.

In other embodiments, exemplary therapeutic agents in the BBP-compositions include, but are not limited to, one or more of the therapeutic agents listed in WO 2013/169739, including the anti-hypertensive and/or a collagen modifying agents (“AHCM”) disclosed, e.g., in Paragraphs 40-49, 283, 286-295; the microenviroment modulators disclosed, e.g., in Paragraphs 113-121, of WO 2013/169739, incorporated herein by reference. In some embodiments, the BBP-composition comprising the AHCM and/or the microenvironment modulator causes one or more of: reduces solid stress (e.g., growth-induced solid stress in tumors); decreases tumor fibrosis; reduces interstitial hypertension or interstitial fluid pressure (IFP); increases interstitial tumor transport: increases tumor or vessel perfusion; increases vascular diameters and/or enlarges compressed or collapsed blood vessels; reduces or depletes one or more of: cancer cells, or stromal cells (e.g., tumor associated fibroblasts or immune cells): decreases the level or production of extracellular matrix components, such as fibers (e.g., collagen, procollagen), and/or polysaccharides (e.g., glycosaminoglycans such as hyaluronan or hyaluronic acid); decreases the level or production of collagen or procollagen; decreases the level or production of hyaluronic acid; increases tumor oxygenation; decreases tumor hypoxia; decreases tumor acidosis; enables immune cell infiltration; decreases immunosuppression; increases antitumor immunity; decreases the production of cancer stem cells (also referred to herein as tumor-initiating cells); or enhances the efficacy (e.g., penetration or diffusion), of the therapy, e.g., the cancer therapy (e.g., radiation, photodynamic therapy, chemotherapeutics, and immunotherapies) in a tumor or tumor vasculature, in the subject.

Agents, e.g., therapeutic agents, include the herein disclosed categories and specific examples. It is not intended that the category be limited by the specific examples. Those of ordinary skill in the art will recognize also numerous other compounds that fall within the categories and that are useful according to the present disclosure.

Examples of therapeutic agents include, but are not limited to, antimicrobial agents, analgesics, antiinflammatory agents, counterirritants, coagulation modifying agents, diuretics, sympathomimetics, anorexics, antacids and other gastrointestinal agents; antiparasitics, antidepressants, anti-hypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anti-cancer properties, or a combination thereof. Other suitable therapeutic agents include contraceptives and vitamins as well as micro- and macronutrients. Still other examples include antiinfectives such as antibiotics and antiviral agents; analgesics and analgesic combinations; anorexics; antiheimintics; antiarthritics; antiasthmatic agents; anticonvulsants; antidepressants; antidiuretic agents; antidiarrleals; antihistamines; antiinflammatory agents; antimigraine preparations antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; anti-hypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

In certain instances, the diagnostic agent is an imaging agent or contrast agent. The terms “imaging agent” and “contrast agent” refer to a substance used to enhance the contrast of structures or fluids within the body in medical imaging. It is commonly used to enhance the visibility of blood vessels and the gastrointestinal tract in medical imaging.

The term “ring-opening metathesis polymerization (ROMP)” refers to a type of olefin metathesis chain-growth polymerization that is driven by the relief of ring strain in cyclic olefins (e.g. norbornene or cyclopentene). The catalysts used in the ROMP reaction include RuCl₃/alcohol mixture, bis(cyclopentadienyl)dimethylzirconium(IV), dichloro[1,3-bis(2,6-isopropylphenyl)-2-imidazolidinylidene](benzylidene)(tricyclohexylphosphine)ruthenium(II), dichloro[1,3-Bis(2-methylphenyl)-2-imidazolidinylidene](benzylidene)(tricyclohexylphosphine) ruthenium(II), dichloro[1,3-bis(2,4,6-trimethylphenyl)-2-imidazolidinylidene][3-(2-pyridinyl)propylidene]ruthenium(II), dichloro(3-methyl-2-butenylidene)bis (tricyclopentylphosphine)ruthenium(I), dichloro[1,3-bis(2-methylphenyl)-2-imidazolidinylidene](2-isopropoxyphenylmethylene)ruthenium(II) (Grubbs C571), dichloro(benzylidene)bis(tricyclohexylphosphine)ruthenium(II) (Grubbs I), dichloro[1,3-bis(2,4,6-trimethylphenyl)-2-imidazolidinylidene](benzylidene)(tricyclohexylphosphine) ruthenium(II) (Grubbs II), and dichloro[1,3-bis(2,4,6-trimethylphenyl)-2-imidazolidinylidene](benzylidene)bis(3-bromopyridine)ruthenium(II) (Grubbs III).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal.

The term “administer,” “administering,” or “administration” refers to implanting, absorbing, ingesting, injecting, inhaling, or otherwise introducing a compound described herein, or a composition thereof, in or on a subject.

The terms “treatment,” “treat.” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease.” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “ratiometric” refers to the situation where C₁ⁱ is substantially equal to C₀ⁱ, wherein C₀ⁱ refers to the ratio of the amount of a first agent before the first agent is delivered to a subject, tissue, or cell, to the total amount of two or more agents (including the first agent) before the two or more agents are delivered to the subject, tissue, or cell; and C₁ⁱ refers to the ratio of the amount of the first agent that is delivered to the subject, tissue, or cell, to the total amount of the two or more agents (including the first agent) that are delivered to the subject, tissue, or cell. In certain embodiments, the delivery of each one of the two or more agents is ratiometric.

The term “orthogonal” refers to the situation where a first agent and a second agent, each of which is included in a BBP described herein, is independently released from the BBP. In certain embodiments, under condition A, the first agent, but not the second agent, is released from the BBP. For example, an orthogonal release or orthogonal delivery of the first and second agents includes: under condition A, the first agent, but not the second agent, is released from the BBP: under condition B, the second agent, but not the first agent, is released from the BBP. The release or delivery of the first and second agents is not orthogonal when, for example, under condition C, both the first and second agents are released from the BBP.

The disclosure is not intended to be limited in any manner by the above exemplary listing of substituents. Additional terms may be defined in other sections of this disclosure.

### DETAILED DESCRIPTION OF CERTAIN EMBODIMENTS

Described herein are compounds (e.g., monomers) that include an agent through a linker that includes a boronic ester moiety in the backbone of the linker. Also provided are polymers prepared by polymerizing the monomers. The polymers may be useful for delivering the agent to a subject, tissue, biological sample, or a cell. In certain embodiments, the polymers are bottlebrush polymers (BBPs). Bottlebrush polymers have found widespread applications in fields ranging from drug delivery and molecular imaging to novel materials and stimuli responsive networks.¹⁻³ Graft-through ring-opening metathesis polymerization (ROMP) offers distinct advantages over other bottlebrush synthesis methods.^{4,5} The fast-initiating Grubbs 3^{rd} generation catalyst (G3-Cat) has been shown to sustain propagation of polymer chain reactions with exceptionally high tolerance towards a wide range of sterically-hindered multivalent monomers (e.g., macromonomers (MMs)), reaching high degrees of polymerization and low dispersity values, even at low millimolar concentrations.^{6,7} Furthermore, using G3-Cat, it is possible to control composition, morphology, and size of final macromolecules, allowing the preparation of remarkable polymeric architectures, such as bottlebrush polymers and star polymers.⁷⁻¹¹ Due to the high packing density of their side-chains, the backbones of bottlebrush polymers may be rigid and adopt extended morphologies with minimal side-chain entanglement.⁶

Also provided are methods of preparing the polymers, compositions and kits comprising the polymers, and methods of use (e.g., use in delivering the agent, treating a disease, preventing a disease, diagnosing a disease) involving the polymers or compositions.

### Compounds, e.g., Monomers

In one aspect, the present disclosure provides compounds. In certain embodiments, the compound is of the formula:

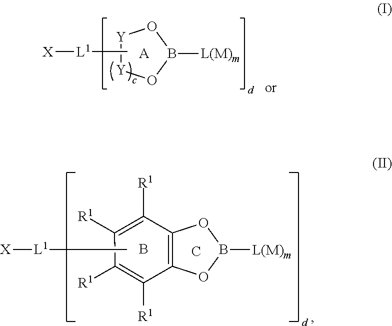

or a salt thereof, wherein:
X is a polymerization handle;
L¹ is a substituted or unsubstituted linker, wherein the backbone of L¹ comprises two or more atoms;
each instance of Y is independently —C(R¹)₂—;
each instance of R¹ is independently absent, hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or two instances of R are joined to form substituted or unsubstituted carbocyclyl or substituted or unsubstituted heterocyclyl;

each instance of R^{a} is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;
each instance of L is independently a bond or a substituted or unsubstituted linker, wherein the atom in the backbone of L attached to Ring A or Ring C is carbon;
each instance of M is independently an agent;
each instance of m is independently an integer between 1 and 10, inclusive;
each instance of c is independently an integer between 1 and 2, inclusive; and
d is an integer between 1 and 10, inclusive.

In certain embodiments, the compound is of Formula (I), or a salt thereof. In certain embodiments, the compound is of Formula (II), or a salt thereof.

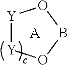

### is Ring A,

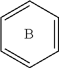

### is Ring B,

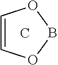

### is Ring C.

In certain embodiments, at least one instance of R¹ in Formula (I) is a carbon-bound moiety.

In certain embodiments, the compound includes a polymerization handle as the moiety X. When the compound includes a polymerization handle as the moiety X, the compound is a monomer. In certain embodiments, X comprises one or more functional groups selected from the group consisting of alkene, vinyl halide, alkyne, amine, —N₃, carboxylic acid, non-aromatic alcohol, and aldehyde. In some embodiments, X comprises one or more functional groups selected from the group consisting of —N₃, —NH₂, —C(═O)OH, —OH, and —C(═O)H. In certain embodiments, X comprises one or more functional groups selected from the group consisting of —NH₂, —C(═O)OH, and —C(═O)H. In some embodiments, X comprises a only one functional group (e.g., alkene, vinyl halide, alkyne, amine, —N₃, carboxylic acid, non-aromatic alcohol, aldehyde, —NH₂, —C(═O)OH, —OH, or —C(═O)H). In certain embodiments. X comprises two or more functional groups (e.g., two or more of alkene, vinyl halide, alkyne, amine, —N₃, carboxylic acid, non-aromatic alcohol, aldehyde, —NH₂, —C(═O)OH, —OH, or —C(═O)H, or a combination thereof). In certain embodiments, X comprises —N₃. In certain embodiments, X is —N₃. In certain embodiments, X is an addition polymerization handle. In some embodiments, X comprises a functional group selected from the group consisting of alkene, alkyne, or vinyl halide. In some embodiments, X comprises alkene. In some embodiments, X comprises alkyne. In certain embodiments. X is a condensation polymerization handle. In some embodiments, X comprises a functional group selected from the group consisting of amine (e.g., primary amine, secondary amine), carboxylic acid, and non-aromatic alcohol (e.g., alkyl alcohol). In some embodiments, X comprises two non-aromatic alcohols, two carboxylic acids, or two amines. In certain embodiments, X is a metathesis polymerization handle. In certain embodiments, X is a ring-opening metathesis polymerization handle. In some embodiments, X comprises substituted or unsubstituted, partially unsaturated carbocyclyl. In some embodiments, X comprises substituted or unsubstituted, partially unsaturated heterocyclyl. In certain embodiments. X is a radical polymerization handle. In certain embodiments, X is a cationic polymerization handle. In certain embodiments. X is an anionic polymerization handle. In certain embodiments, X comprises substituted or unsubstituted styrene, substituted or unsubstituted acrylate, substituted or unsubstituted methacrylate, substituted or unsubstituted cyclooctene, or substituted or unsubstituted maleimide. In certain embodiments, X comprises an alkyl acrylate, hydroxyalkyl acrylate, haloalkyl acrylate, polymethacrylate, alkyl methacrylate, hydroxyalkyl methacrylate, or haloalkyl methacrylate. In certain embodiments, X comprises ethylene, tetrafluoroethylene, propylene, isobutylene, styrene, acrylonitrile, vinyl chloride, methyl acrylate, methyl methacrylate, butadiene, chloroprene, cis-1,4-isoprene, or trans-1,4-isoprene. In certain embodiments, X comprises ethylene. In certain embodiments, X comprises styrene. In certain embodiments, X comprises methyl methacrylate. In certain embodiments, X comprises an amide, aramide, ester, carbonate, or silicone.

In certain embodiments, X does not comprise a hydroxy-(substituted or unsubstituted phenyl) group. In certain embodiments, X does not comprise a hydroxy-(substituted phenyl) group.

In certain embodiments, X is of the formula:

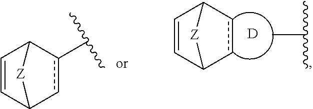

wherein:
Z is C(R^{P})₂ or O;
each instance of R^{P} is independently hydrogen, halogen, or substituted or unsubstituted, C₁₋₆ alkyl;
[symbol] is a single or double bond; and

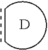

is Ring D, wherein Ring D is a substituted or unsubstituted, monocyclic carbocyclic ring, substituted or unsubstituted, monocyclic heterocyclic ring, substituted or unsubstituted, monocyclic aryl ring, or substituted or unsubstituted, monocyclic heteroaryl ring.

In certain embodiments. Z is O. In certain embodiments Z is C(R^{P})₂. In certain embodiments Z is CH₂. In certain embodiments Z is C(CH₃)₂.

In certain embodiments, each instance of R^{P} is hydrogen. In some embodiments, at least one instance of R^{P} is hydrogen. In certain embodiments, each instance of R^{P} is halogen. In some embodiments, at least one instance of R^{P} is halogen. In certain embodiments, at least one instance of R^{P} is unsubstituted, C₁₋₆ alkyl. In some embodiments, at least one instance of R^{P} is substituted, C₁₋₆ alkyl. In certain embodiments, each instance of R^{P} is unsubstituted methyl. In some embodiments, at least one instance of R^{P} is unsubstituted methyl.

In some embodiments, [symbol] is a single bond. In certain embodiments, [symbol] is a double bond.

In certain embodiments, Ring D is a substituted or unsubstituted, monocyclic carbocyclic ring. In some embodiments, Ring D is an unsubstituted monocyclic heterocyclic ring. In some embodiments, Ring D is a substituted monocyclic heterocyclic ring. In certain embodiments, Ring D is a 5-membered nitrogen containing ring. In some embodiments, Ring D is a 5-membered nitrogen containing ring substituted with oxo (═O). In some embodiments, Ring D is a 5-membered nitrogen containing ring substituted with two instances of oxo.

In some embodiments, X is of the formula

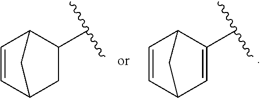 [SMILES: *C1CC2C=CC1C2 ; *C1=CC2C=CC1C2]

In some embodiments, X is of the formula

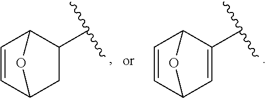 [SMILES: *C1CC2C=CC1O2 ; *C1=CC2C=CC1O2]

In certain embodiments, X is of the formula

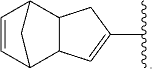 [SMILES: *C1=CC2C3C=CC(C3)C2C1]

In some embodiments, X is of the formula

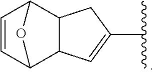 [SMILES: *C1=CC2C3C=CC(O3)C2C1]

In certain embodiments, X is of the formula:

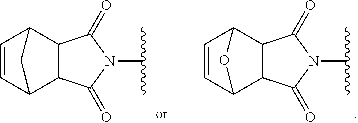 [SMILES: *N1C(=O)C2C3C=CC(C3)C2C1=O ; *N1C(=O)C2C3C=CC(O3)C2C1=O]

In some embodiments, X is of the formula:

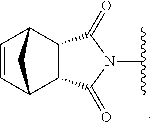 [SMILES: *N1C(=O)[C@@H]2[C@H](C1=O)[C@@H]1C=C[C@H]2C1]

In some embodiments, X is of the formula:

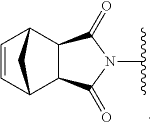 [SMILES: *N1C(=O)[C@@H]2[C@H](C1=O)[C@H]1C=C[C@@H]2C1]

In certain embodiments. X is of the formula:

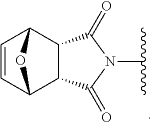 [SMILES: *N1C(=O)[C@@H]2[C@H](C1=O)[C@@H]1C=C[C@H]2O1]

In some embodiments, X is of the formula:

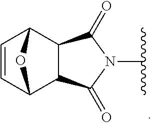

The compound described herein includes the linker L¹. In certain embodiments, L¹ is unsubstituted or substituted, C₂₋₃₀₀ (e.g., C₁₀₋₄₀) alkylene. In certain embodiments, L¹ is unsubstituted or substituted, C₂₋₃₀₀ (e.g., C₁₀₋₄₀) alkenylene. In certain embodiments, L¹ is unsubstituted or substituted, C₂₋₃₀₀ (e.g., C₁₀₋₄₀) alkynylene. In certain embodiments, L¹ is unsubstituted or substituted, C₂₋₃₀₀ (e.g., C₁₀₋₄₀) heteroalkylene. In certain embodiments. L¹ is unsubstituted or substituted, C₂₋₃₀₀ heteroalkenylene. In certain embodiments, L¹ is unsubstituted or substituted, C₂₋₃₀₀ (e.g., C₁₀₋₄₀) heteroalkynylene.

In certain embodiments, no backbone carbon atoms of L¹ are replaced with substituted or unsubstituted carbocyclene, substituted or unsubstituted heterocyclene, substituted or unsubstituted arylene, or substituted or unsubstituted heteroarylene. In some embodiments, 1 or more backbone carbon atoms of L¹ are independently replaced with a substituted or unsubstituted heteroarylene. In some embodiments, 1 or more backbone carbon atoms of L¹ are independently replaced with a substituted or unsubstituted arylene. In some embodiments, one or more backbone carbon atom of L¹ is independently replaces by a substituted or unsubstituted heteroarylene and a substituted or unsubstituted arylene.

In certain embodiments. L¹ is unsubstituted or substituted, C₂₋₃₀₀ (e.g., C₁₀₋₄₀) alkylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments, L¹ is unsubstituted or substituted, C₂₋₃₀₀ (e.g., C₁₀₋₄₀) alkenylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments. L¹ is unsubstituted or substituted, C₂₋₃₀ (e.g., C₁₀₋₄₀) alkynylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments. L¹ is unsubstituted or substituted, C₂₋₃₀₀ (e.g., C₁₀₋₄₀) heteroalkylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments. L¹ is unsubstituted or substituted, C₂₋₃₀₀ (e.g., C₁₀₋₄₀) heteroalkenylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments, L¹ is unsubstituted or substituted, C₂₋₃₀₀ (e.g., C₁₀₋₄₀) heteroalkynylene, wherein 0, 1, 2, 3, or more backbone carbon atoms thereof are independently replaced with substituted or unsubstituted carbocyclene, substituted or unsubstituted heterocyclene, substituted or unsubstituted arylene, or substituted or unsubstituted heteroarylene. In certain embodiments. L¹ is unsubstituted or substituted, C₂₋₃₀₀ (e.g., C₁₀₋₄₀) alkylene, wherein at least one instance of the backbone carbon atom thereof is replaced with substituted or unsubstituted heteroarylene

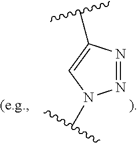 [SMILES: *c1cn(*)nn1]

In certain embodiments, L¹ is unsubstituted or substituted, C₂₋₃₀₀ (e.g., C₁₀₋₄₀) alkenylene, wherein at least one instance of the backbone carbon atom thereof is replaced with substituted or unsubstituted heteroarylene

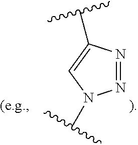 [SMILES: *c1cn(*)nn1]

In certain embodiments, L¹ is unsubstituted or substituted, C₂₋₃₀₀ (e.g., C₁₀₋₄₀) alkynylene, wherein at least one instance of the backbone carbon atom thereof is replaced with substituted or unsubstituted heteroarylene

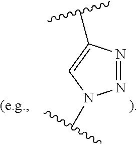 [SMILES: *c1cn(*)nn1]

In certain embodiments, L¹ is unsubstituted or substituted, C₂₋₃₀₀ (e.g., C₁₀₋₄₀) heteroalkylene, wherein at least one instance of the backbone carbon atom thereof is replaced with substituted or unsubstituted heteroarylene

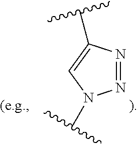 [SMILES: *c1cn(*)nn1]

In certain embodiments, L¹ is unsubstituted or substituted, C₂₋₃₀₀ (e.g., C₁₀₋₄₀) heteroalkenylene, wherein at least one instance of the backbone carbon atom thereof is replaced with substituted or unsubstituted heteroarylene

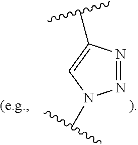 [SMILES: *c1cn(*)nn1]

In certain embodiments. L¹ is unsubstituted or substituted, C₂₋₃₀₀ (e.g., C₁₀₋₄₀) heteroalkynylene, wherein at least one instance of the backbone carbon atom thereof is replaced with substituted or unsubstituted heteroarylene

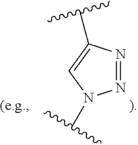 [SMILES: *c1cn(*)nn1]

In certain embodiments. L¹ is substituted or unsubstituted, C₁₀₋₄₀ alkylene, or substituted or unsubstituted, C₁₀₋₄₀ heteroalkylene, wherein at least one instance of the backbone carbon atom thereof is replaced with

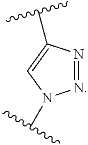 [SMILES: *c1cn(*)nn1]

In some embodiments, L¹ is substituted with substituted or unsubstituted, C₂₋₂₀₀ alkylene or substituted or unsubstituted, C₂₋₂₀₀ heteroalkylene. In certain embodiments. L¹ is substituted with substituted, C₂₋₂₀₀ heteroalkylene. In certain embodiments, L¹ is substituted with C₂₋₂₀₀ heteroalkylene comprising at least one nitrogen atom and at least one oxygen atom. In certain embodiments, L¹ is substituted with C₂₋₂₀₀ heteroalkylene comprising at least one nitrogen atom and one or more oxygen atom. In certain embodiments, L¹ is substituted with oxo-substituted, C₂₋₂₀₀ heteroalkylene comprising at least one nitrogen atom and at least one oxygen atom. In certain embodiments, L¹ is substituted with oxo-substituted, C₂₋₂₀₀ heteroalkylene comprising at least one nitrogen atom and one or more oxygen atom.

In certain embodiments, L¹ comprises a polymer. In some embodiments, L¹ comprises substituted or unsubstituted polyethylene. In some embodiments, L¹ comprises unsubstituted polyethylene. In some embodiments, L¹ comprises substituted or unsubstituted polystyrene. In some embodiments, L¹ comprises PEG. In certain embodiments. L¹ comprises a polymer (e.g., PEG) with a weight-average molecular weight between 200 and 500, between 500 and 1,000, between 0,000 and 2,000, between 2,000 and 5,000, between 5,000 and 10,000, or between 10,000 and 50,000, inclusive, g/mol. In some embodiments, L¹ comprises a polymer (e.g., PEG) with the weight average molecular weight between 1,000 and 5,000, inclusive, g/mol. In some embodiments, L¹ comprises a polymer (e.g., PEG) with the weight average molecular weight between 2,000 and 5,000, inclusive, g/mol.

The compound of any one of the preceeding claims, or salt thereof, wherein L¹ is of the formula:

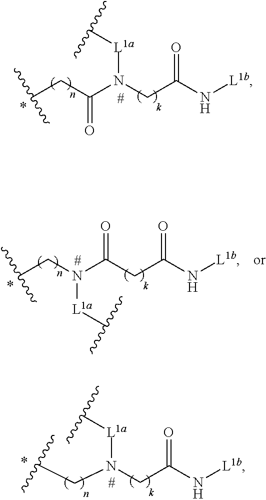

wherein:
n is an integer between 0 and 12 inclusive;
k is an integer between 1 and 12 inclusive;
L^{1a} is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or C₂₋₂₀₀ heteroalkynylene, wherein:
optionally one or more backbone carbon atoms in each instance of the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclene, substituted or unsubstituted heterocyclene, substituted or unsubstituted arylene, or substituted or unsubstituted heteroarylene;
optionally one or more backbone heteroatoms in each instance of the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclene, substituted or unsubstituted heterocyclene, substituted or unsubstituted arylene, or substituted or unsubstituted heteroarylene;
L^{1b} is substituted or unsubstituted, C₁₋₂₀₀ alkyl, substituted or unsubstituted, C₂₋₂₀₀ alkenyl, substituted or unsubstituted, C₂₋₂₀₀ alkynyl, substituted or unsubstituted, C₂₋₂₀₀ heteroalkyl, substituted or unsubstituted, C₂₋₂₀₀ heteroalkenyl, C₂₋₂₀₀ heteroalkynyl, substituted or unsubstituted carbocyclyl, substituted or unsubstituted heterocyclyl, substituted or unsubstituted aryl, substituted or unsubstituted heteroaryl, substituted or unsubstituted acyl, a nitrogen protecting group, a polymer, a peptide, or a protein: and
the attachment point labeled with “*” is attached to X and the other attachment point is attached to Ring A of Formula I or Ring B of Formula II.

In certain embodiments, L¹ is of the formula:

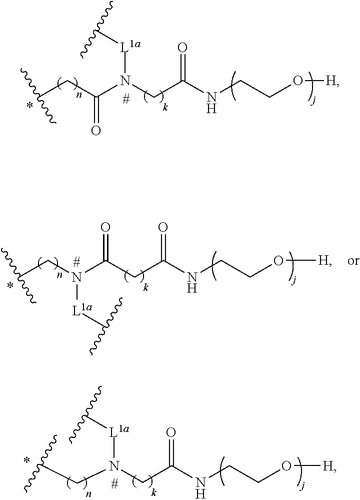

wherein:
n is an integer between 0 and 12 inclusive;
k is an integer between 1 and 12 inclusive;
j is an integer between 0 and 300 inclusive; and
L^{1a} is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or C₂₋₂₀₀ heteroalkynylene, wherein:
optionally one or more backbone carbon atoms in each instance of the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclene, substituted or unsubstituted heterocyclene, substituted or unsubstituted arylene, or substituted or unsubstituted heteroarylene;
optionally one or more backbone heteroatoms in each instance of the substituted or unsubstituted, C₂₋₂₀₀ heteroalkylene, substituted or unsubstituted C₂₋₂₀₀ heteroalkenylene, and substituted or unsubstituted, C₂₋₂₀₀ heteroalkynylene are independently replaced with substituted or unsubstituted carbocyclene, substituted or unsubstituted heterocyclene, substituted or unsubstituted arylene, or substituted or unsubstituted heteroarylene; and
the attachment point labeled with “*” is attached to X and the other attachment point is attached to Ring A of Formula I or Ring B of Formula II.

In certain embodiments. L is of the formula

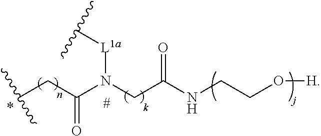

In some embodiments, L is of the formula

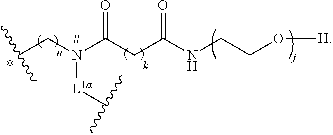

In certain embodiments. L¹ is of the formula

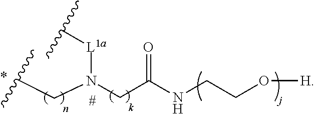

In certain embodiments. L¹ is of the formula:

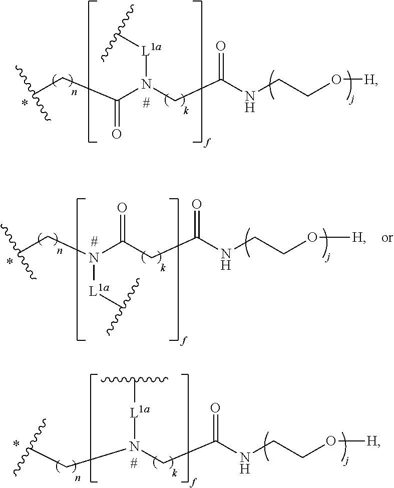

wherein:
f is an integer between 1 and 10 inclusive; and
the attachment point labeled with “*” is attached to X and the other attachment point is attached to Ring A of Formula I or Ring B of Formula II.

In some embodiments, n is 3. In certain embodiments, n is 4. In some embodiments, n is 5. In some embodiments, n is 6.

In some embodiments, at least one instance of k is 1. In some embodiments, at least one instance of k is 2. In some embodiments, at least one instance of k is 3 or 4.

In some embodiments, j is between 10 and 200, inclusive. In some embodiments, j is between 20 and 150, inclusive. In some embodiments, j is between 40 and 100, inclusive. In some embodiments, j is between 60 and 80, inclusive.

In some embodiments, f is 1. In some embodiments, f is 2. In some embodiments, f is 3.

In some embodiments, n is 4, each instance of k is 1, j is between 60 and 80, inclusive, and f is 1. In some embodiments, n is 5, each instance of k is 1, j is between 60 and 80, inclusive, and f is 1. In some embodiments, n is 4, each instance of k is 2, j is between 60 and 80, inclusive, and f is 1. In some embodiments, n is 5, each instance of k is 2, j is between 60 and 80, inclusive, and f is 1.

In some embodiments, L is of the formula

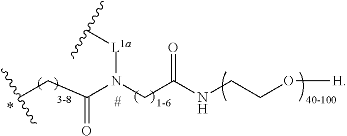

In some embodiments, L¹ is of the formula

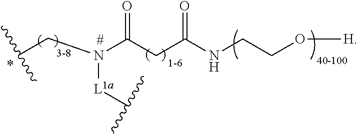

In some embodiments, L¹ is of the formula

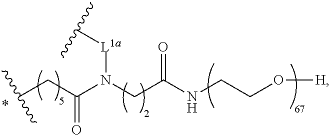

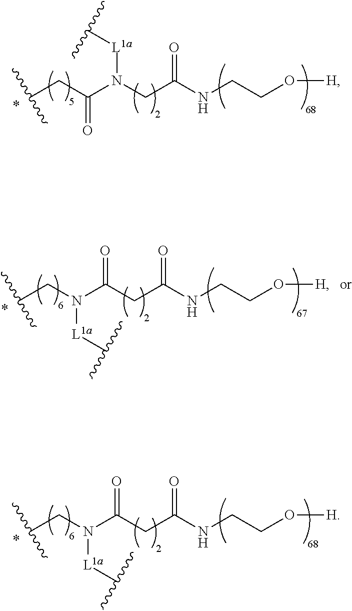

In some embodiments, at least one instance of L^{1a} is substituted, C₂₋₂₀₀ alkylene. In some embodiments, at least one instance of L^{1a} is substituted or unsubstituted, C₂₋₁₀₀ alkylene. In some embodiments, at least one instance of L^{1a} is substituted or unsubstituted, C₂₋₁₀₀ alkylene wherein one or more backbone carbon atoms are independently replaced with an oxygen atom, substituted or unsubstituted nitrogen atom (e.g., —N(H)—, —N(CH₃)—), substituted or unsubstituted heteroarylene, or substituted or unsubstituted arylene. In some embodiments, at least one instance of L^{1a} is substituted or unsubstituted, C₂₋₁₀₀ alkylene wherein one or more backbone carbon atoms are replaced with an oxygen atom, one or more backbone carbon atoms are replaced with substituted or unsubstituted nitrogen atom (e.g., —N(H)—, —N(CH₃)—), one or more backbone carbon atoms are replaced with substituted or unsubstituted heteroarylene, or one or more backbone carbon atoms are replaced with substituted or unsubstituted arylene. In some embodiments, at least one instance of L is substituted or unsubstituted. C₂₋₁₀₀ alkylene wherein one or more backbone carbon atoms are replaced with substituted or unsubstituted heteroarylene. In some embodiments, at least one instance of L^{1a} is substituted or unsubstituted. C₂₋₁₀₀ alkylene wherein one or more backbone carbon atoms are replaced with an oxygen atom and one or more backbone carbon atoms are replaced with substituted or unsubstituted heteroarylene. In some embodiments, at least one instance of L is an oxo-substituted C₂₋₁₀₀ alkylene wherein one or more backbone carbon atoms are replaced with an oxygen atom, one or more backbone carbon atoms are replaced with substituted or unsubstituted nitrogen (e.g., —N(H)—, —N(CH₃)—), one or more backbone carbon atoms are replaced with substituted or unsubstituted heteroarylene, or one or more backbone carbon atoms are replaced with substituted or unsubstituted arylene. In some embodiments, at least one instance of L is substituted or unsubstituted, C₂₋₁₀₀ alkylene wherein one or more backbone carbon atoms are replaced with an oxygen atom, one or more backbone carbon atoms are replaced with substituted or unsubstituted heteroarylene, or one or more backbone carbon atoms are replaced with substituted or unsubstituted arylene.

In some embodiments, at least one instance of L^{1a} is substituted, C₂₋₂₀₀ heteroalkylene. In some embodiments, at least one instance of L is substituted or unsubstituted, C₂₋₁₀₀ heteroalkylene. In some embodiments, at least one instance of L is substituted or unsubstituted, C₂₋₁₀₀ heteroalkylene wherein one or more backbone carbon atoms are replaced with substituted or unsubstituted arylene. In some embodiments, at least one instance of L^{1a} is substituted or unsubstituted, C₂₋₂₀₀ (e.g., C₂₋₁₀₀) heteroalkylene wherein one or more backbone carbon atoms are replaced with substituted or unsubstituted heteroarylene. In certain embodiments, at least one instance of L^{1a} is a substituted or unsubstituted, C₂₋₂₀₀ (e.g., C₂₋₁₀₀) heteroalkylene wherein one or more backbone carbon atoms are replaced with

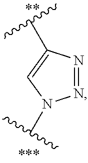 [SMILES: *c1cn(*)nn1]

wherein “**” is attached closer to the nitrogen atom labeled “#”, and “***” is attached closer to either Ring A of Formula I or Ring B of Formula II. In certain embodiments, at least one instance of L^{1a} is a substituted or unsubstituted, C₂₋₁₀₀ heteroalkylene wherein one or more backbone carbon atoms are independently replaced with

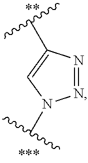 [SMILES: *c1cn(*)nn1]

wherein “**” is attached closer to the nitrogen atom labeled “#” and “***” is attached closer to either Ring A of Formula I or Ring B of Formula II. In certain embodiments, at least one instance of L^{1a} is a substituted or unsubstituted, C₂₋₁₀₀ heteroalkylene wherein one backbone carbon atom is replaced with

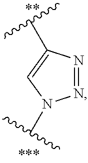 [SMILES: *c1cn(*)nn1]

wherein “**” is attached closer to the nitrogen atom labeled “#” and “***” is attached closer to either Ring A of Formula I or Ring B of Formula II.

In certain embodiments, at least one instance of L^{1a} is of the formula

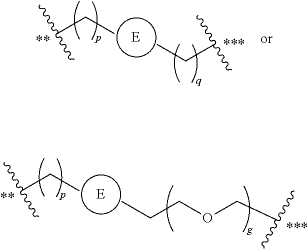

wherein:
each instance of p is independently an integer between 0 and 12 inclusive;
each instance of q is independently an integer between 0 and 12 inclusive;
each instance of Ring E is independently a substituted or unsubstituted carbocyclene, substituted or unsubstituted heterocyclene, substituted or unsubstituted arylene, or substituted or unsubstituted heteroarylene;
each instance of g is independently an integer between 0 and 12 inclusive; and
“**” is attached closer to the nitrogen atom labeled “#” and “***” is attached closer to either Ring A of Formula I or Ring B of Formula II.

In certain embodiments, at least one instance of p is 0. In certain embodiments, at least one instance of p is 1. In certain embodiments, at least one instance of p is 2. In certain embodiments, at least one instance of p is 3, 4, 5, or 6. In certain embodiments, at least one instance of p is an integer from 7 to 12, inclusive.

In certain embodiments, at least one instance of q is 0. In certain embodiments, at least one instance of q is 1. In certain embodiments, at least one instance of q is 2. In certain embodiments, at least one instance of q is 3. In certain embodiments, at least one instance of q is 4, 5, or 6. In certain embodiments, at least one instance of q is an integer from 7 to 12, inclusive.

In certain embodiments, at least one instance of Ring E is substituted or unsubstituted carbocyclene (e.g., substituted or unsubstituted, 3- to 7-membered, monocyclic carbocyclene). In certain embodiments, at least one instance of Ring E is substituted or unsubstituted heterocyclene (e.g., substituted or unsubstituted, 3- to 7-membered, monocyclic heterocyclene). In certain embodiments, at least one instance of Ring E is substituted or unsubstituted arylene. In certain embodiments, at least one instance of Ring E is substituted or unsubstituted phenylene (e.g., 1,4-phenylene). In certain embodiments, at least one instance of Ring E is substituted or unsubstituted heteroarylene (e.g., substituted or unsubstituted, 5- to 6-membered, monocyclic heteroarylene). In certain embodiments, at least one instance of Ring E is substituted or unsubstituted 1,2,3-triazolylene (e.g.,

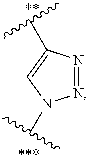 [SMILES: *c1cn(*)nn1]

wherein “**” is attached closer to the nitrogen atom labeled “#” and “***” is attached closer to either Ring A of Formula I or Ring B of Formula II.)

In certain embodiments, at least one instance of g is 0. In certain embodiments, at least one instance of g is 1. In certain embodiments, at least one instance of g is 2. In certain embodiments, at least one instance of g is 3. In certain embodiments, at least one instance of g is 3, 4, or 5. In certain embodiments, at least one instance of g is an integer from 6 to 12, inclusive.

In some embodiments, at least one instance of L^{1a} is of the formula

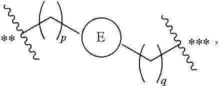

at least one instance of optionally wherein at least one instance of R in at least one instance of g E is

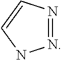

In some embodiments, at least one instance of L^{1a} is of the formula

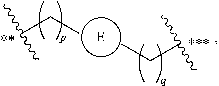

at least one instance of optionally wherein at least one instance of R in at least one instance of g E is

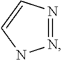

at least one instance of p is between 1 and 2 inclusive, and at least one instance of q is between 4 and 12 inclusive.

In certain embodiments, at least one instance of L^{1a} is of the formula

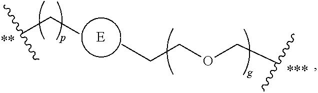

at least one instance of optionally wherein at least one instance of R in at least one instance of g E is

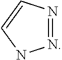

In some embodiments, at least one instance of L^{1a} is of the formula

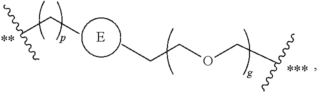

at least one instance of optionally wherein at least one instance of R in at least one instance of g E is

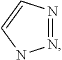

and at least one instance of p is between 1 or 2 inclusive. In some embodiments, at least one instance of L^{1a} is of the formula

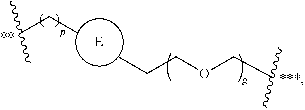

at least one instance of optionally wherein at least one instance of R in at least one instance of g E is

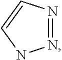

at least one instance of p is between 1 or 2 inclusive, and at least one instance of g is 3, 4, or 5.

In some embodiments, at least one instance of L^{1a} is of the formula:

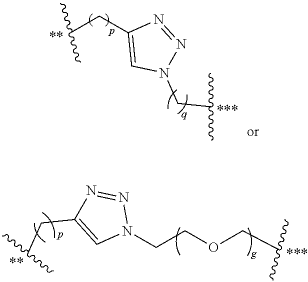

wherein:
“**” is attached closer to the nitrogen atom labeled “#” and “***” is attached closer to either Ring A of Formula I or Ring B of Formula II.

In some embodiments, at least one instance of L^{1a} is of the formula

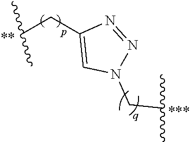

wherein p is 1 or 2. In some embodiments, at least one instance of L^{1a} is of the formula

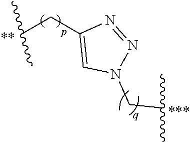

wherein p is 1 or 2 and q is between 8 and 12 inclusive. In some embodiments, at least one instance of L^{1a} is of the formula

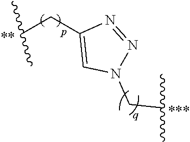

wherein p is 1 or 2 and q is between 1 and 3 inclusive. In certain embodiments, at least one instance of L^{1a} is of the formula

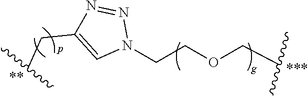

wherein p is 1 or 2. In certain embodiments, at least one instance of L^{1a} is of the formula

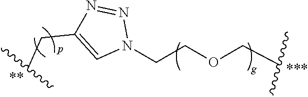

wherein p is 1 or 2 and g is 3, 4, or 5. In certain embodiments, at least one instance of L^{1a} is of the formula

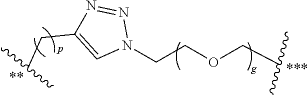

wherein p is 1 or 2 and g is 3, 4, or 5.

In certain embodiments, at least one instance of L^{1a} is of the formula:

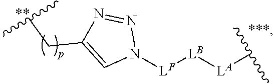

wherein:
each instance of p is independently an integer from 1 to 12, inclusive;
each instance of L^{F} is independently substituted or unsubstituted, C₂₋₁₈₀ heteroalkylene;
each instance of -L^{B}-L^{A}- is independently —C(═O)O—, —OC(═O)—, —C(═O)NR^{E}, —NR^{E}C(═O)—, or a single bond, wherein each instance of R^{E} is independently hydrogen, substituted or unsubstituted, C₁₋₆ alkyl, or a nitrogen protecting group; and
“**” is attached closer to the nitrogen atom labeled “#” and “***” is attached closer to either Ring A of Formula I or Ring B of Formula II.

In certain embodiments, at least one instance of L^{1a} is of the formula

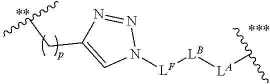

wherein L^{F} is substituted or unsubstituted, C₅₋₂₀ heteroalkylene. In certain embodiments, at least one instance of L^{1a} is of the formula

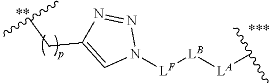

wherein L^{F} is substituted or unsubstituted, C₅₋₂₀ heteroalkylene comprising in the backbone thereof carbon and oxygen atoms. In certain embodiments, at least one instance of L^{1a} is of the formula

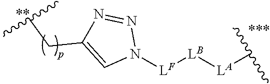

wherein p is 1 or 2, L^{F} is substituted or unsubstituted, C₅₋₂₀ heteroalkylene comprising in the backbone thereof carbon and oxygen atoms, and -L^{B}-L^{A}- is —CH₂—.

In some embodiments, at least one instance of L^{1a} is of the formula:

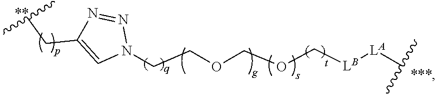

wherein:
each instance of p is independently an integer from 1 to 12, inclusive;
each instance of q is independently an integer from 1 to 12, inclusive;
each instance of g is independently an integer from 0 to 12, inclusive;
each instance of s is independently 0 or 1;
each instance of t is independently an integer from 0 to 10, inclusive;
each instance of -L^{B}-L^{A}- is independently —C(═O)O—, —OC(═O)—, —C(═O)NR^{E}—, —NR^{E}C(═O)—, or a single bond, wherein each instance of R^{E} is independently hydrogen, substituted or unsubstituted, C₁₋₆ alkyl, or a nitrogen protecting group; and
“**” is attached closer to the nitrogen atom labeled “#” and “***” is attached closer to either Ring A of Formula I or Ring B of Formula II.

In certain embodiments, at least one instance of L^{1a} is of the formula

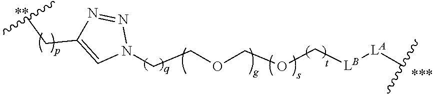

wherein p is between 1 and 2 inclusive, q is 1, g is between 2 and 10 inclusive. In certain embodiments, at least one instance of L^{1a} is of the formula

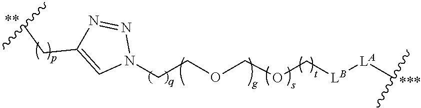

wherein p is 1, q is 1, g is between 3, 4, or 5, and s is 0. In certain embodiments, at least one instance of L¹ is of the formula

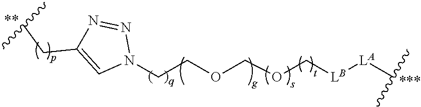

wherein p is 1 or 2 inclusive, q is 1, g is 3, 4, or 5, s is 0, and -L^{B}-L^{A}- is a bond.

In some embodiments, at least one instance of L^{1a} is of the formula:

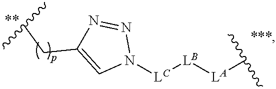

wherein:
each instance of p is independently an integer from 1 to 12, inclusive;
each instance of L^{C} is independently substituted or unsubstituted, C₁₋₁₈₀ alkylene;
each instance of -L^{B}-L^{A}- is independently —C(═O)O—, —OC(═O)—, —C(═O)NR^{E}—, —NR^{E}C(═O)—, or a single bond, wherein each instance of R^{E} is independently hydrogen, substituted or unsubstituted, C₁₋₆ alkyl, or a nitrogen protecting group; and
“**” is attached closer to the nitrogen atom labeled “#” and “***” is attached closer to either Ring A of Formula I or Ring B of Formula II.

In certain embodiments, at least one instance of L^{a} is of the formula

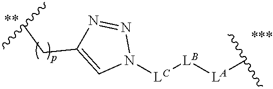

wherein L is substituted or unsubstituted, C₅₋₂₀ alkylene. In certain embodiments, at least one instance of L^{1a} is of the formula

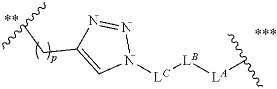

wherein L^{C} is C₅₋₂₀ alkylene substituted with at least one instance of substituted or unsubstituted phenyl or substituted or unsubstituted, C₁₋₆ alkyl. In certain embodiments, at least one instance of L^{1a} is of the formula

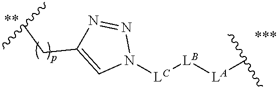

wherein L^{C} is C₅₋₂₀ alkylene substituted with at least one instance of substituted or unsubstituted phenyl. In certain embodiments, at least one instance of L^{1a} is of the formula

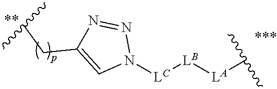

wherein L^{C} is C₅₋₂₀ alkylene substituted with at least one instance of substituted or unsubstituted, C₁₋₆ alkyl. In certain embodiments, at least one instance of L^{1a} is of the formula

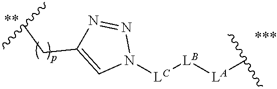

wherein p is 1 or 2. L^{C} is substituted or unsubstituted, C₅₋₂₀ alkylene, and -L^{B}-L^{A}- is —C(═O)O—.

In certain embodiments. L^{1b} is substituted, C₁₋₂₀₀ alkyl. In certain embodiments, L^{1b} is C₁₋₂₀₀ alkyl substituted at least with oxo. In some embodiments, L^{1b} is substituted, C₂₋₂₀₀ heteroalkyl. In certain embodiments, L^{1b} is substituted, C₂₋₂₀₀ heteroalkyl comprising in the backbone thereof one or more oxygen atoms. In certain embodiments, L^{1b} is substituted, C₂₋₂₀₀ heteroalkyl comprising in the backbone thereof one or more oxygen atoms. In certain embodiments. Lib comprises a polymer. In certain embodiments, L^{1b} comprises a polymer (e.g., PEG) with a weight-average molecular weight between 200 and 500, between 500 and 1,000, between 1,000 and 2,000, between 2,000 and 5,000, between 5,000 and 10,000, or between 10,000 and 50,000, inclusive, g/mol. In some embodiments, L^{1b} comprises a polymer (e.g., PEG) with the weight average molecular weight between 1,000 and 5,000, inclusive, g/mol. In some embodiments, L^{1b} comprises a polymer (e.g., PEG) with the weight average molecular weight between 2,000 and 5,000, inclusive, g/mol. In some embodiments, L^{1b} is PEG (e.g., PEG with the weight average molecular weight between 1,000 and 5,000).

In certain embodiments. L^{1b} comprises a zwitterionic unit. In certain embodiments, L^{1b} is a zwitterionic unit. In certain embodiments, L^{1b} comprises a hydrophilic moiety (e.g., a small-molecule hydrophilic moiety). In certain embodiments. L^{1b} is a hydrophilic moiety (e.g., a small-molecule hydrophilic moiety).

In certain embodiments, each R¹ is the same. In some embodiments, each R¹ is different. In some embodiments, some instances of R¹ are the same and some instances of R¹ are different. In some embodiments, each R¹ is independently substituted or unsubstituted 3- to 12-membered carbocyclyl (e.g., cyclopentyl, cyclohexyl, cyclopropyl) or substituted or unsubstituted 3- to 12-membered heterocyclyl (e.g., morpholinyl, piperdinyl, pyrrolinyl, tetrahydrofuranyl, tetrahydropyranyl, piperizinyl). In some embodiments, each R¹ is independently substituted or unsubstituted 5- to 10-membered aryl (e.g., phenyl, naphthalyl), and substituted or unsubstituted 5- to 10-membered heteroaryl (e.g., pyridinyl, pyrimidinyl).

In certain embodiments, each instance of R¹ is independently absent, hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, or substituted or unsubstituted heteroaryl, or two instances of R¹ are joined to form substituted or unsubstituted carbocyclyl or substituted or unsubstituted heterocyclyl.

In certain embodiments, no instance of R¹ is hydrogen. In some embodiments, some instances of R¹ are hydrogen.

In some embodiments, each R¹ is independently substituted or unsubstituted. C₁₋₁₂ alkyl. In certain embodiments, each R¹ is independently substituted or unsubstituted, C₁₋₆ alkyl. In some embodiments, each R¹ is independently substituted or unsubstituted methyl, substituted or unsubstituted ethyl, substituted or unsubstituted propyl, substituted or unsubstituted butyl, substituted or unsubstituted pentyl, and substituted or unsubstituted hexyl. In some embodiments, each R¹ is independently unsubstituted, C₁₋₆ alkyl (e.g., methyl, ethyl, propyl, isopropyl). In some embodiments, each R¹ is independently unsubstituted methyl. In some embodiments, each instance of R¹ is unsubstituted methyl. In some embodiments, R¹ is unsubstituted. C₁₋₆ alkyl trifluoromethyl, fluoromethyl).

In certain embodiments, two instances of R¹ are joined to form substituted or unsubstituted carbocyclyl or substituted or unsubstituted heterocyclyl. When two instances of R¹ are joined to form substituted or unsubstituted carbocyclyl or substituted or unsubstituted heterocyclyl. L¹ may be attached to Ring A, Ring B, or the carbocyclyl or heterocyclyl formed by joining two instances of R. In certain embodiments, two instances of R¹ are joined to form substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, two instances of R¹ are joined to form substituted or unsubstituted cyclohexyl. In certain embodiments, two instances of R¹ are joined to form substituted or unsubstituted, monocyclic, 3- to 7-membered heterocyclyl (e.g., substituted or unsubstituted morpholinyl, substituted or unsubstituted piperdinyl, substituted or unsubstituted pyrrolinyl, substituted or unsubstituted tetrahydrofuranyl, substituted or unsubstituted tetrahydropyranyl, or substituted or unsubstituted piperizinyl).

In some embodiments,

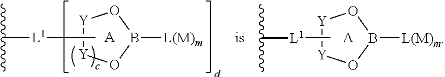

In certain embodiments,

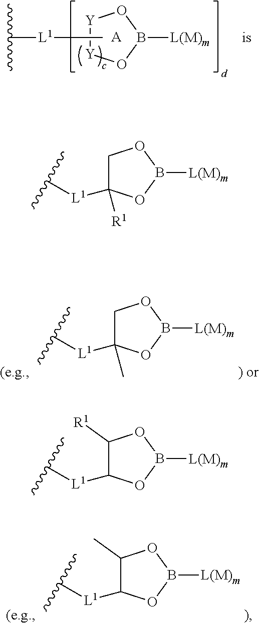

provided that R¹ is not absent or H. In certain embodiments,

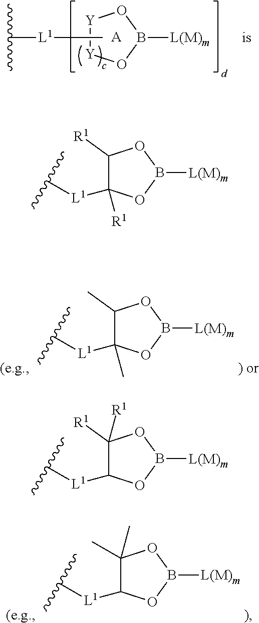

provided that each instance of R¹ is not absent or H. In certain embodiments,

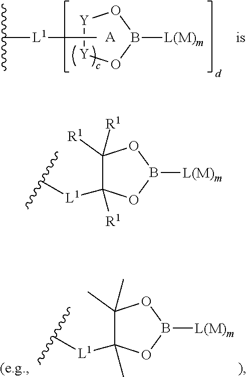

provided that each instance of R¹ is not absent or H.

In certain embodiments,

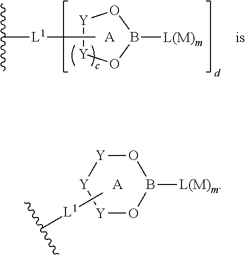

In certain embodiments,

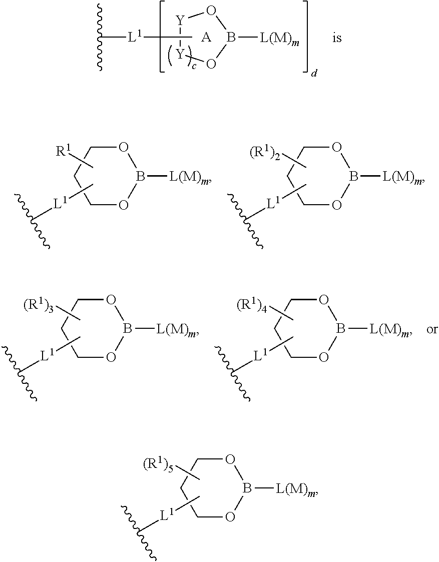

provided that each instance of R is not absent or H. In some embodiments,

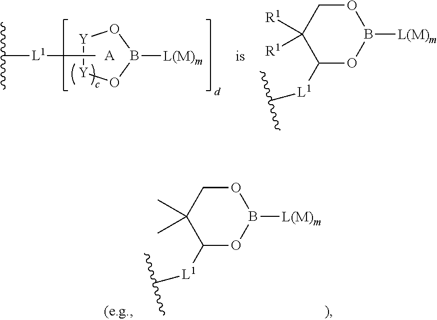

provided that each instance of R is not absent or H. In certain embodiments,

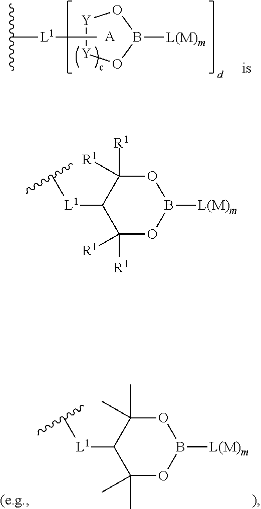

provided that each instance of R¹ is not absent or H.

In certain embodiments,

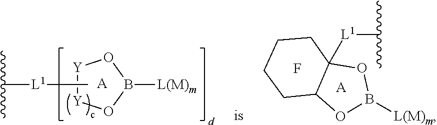

wherein Ring A and Ring F are independently substituted (e.g., independently substituted with one or more instances of substituted or unsubstituted alkyl) or unsubstituted. In certain embodiments,

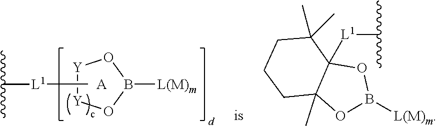

In some embodiments,

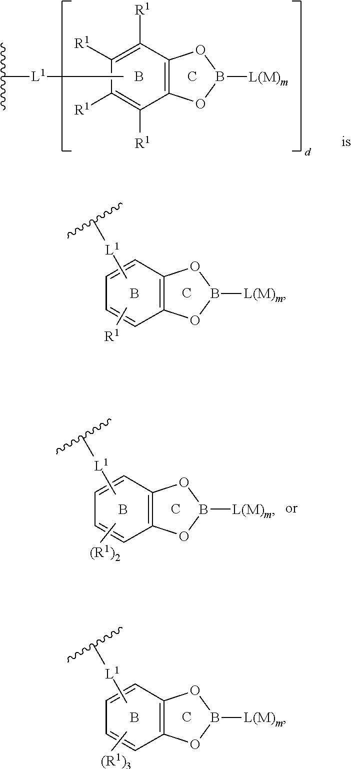

provided that no instance of R¹ is absent or hydrogen.

In certain embodiments,

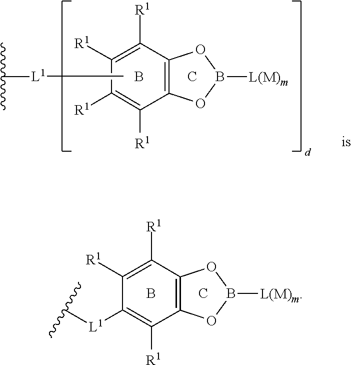

In some embodiments,

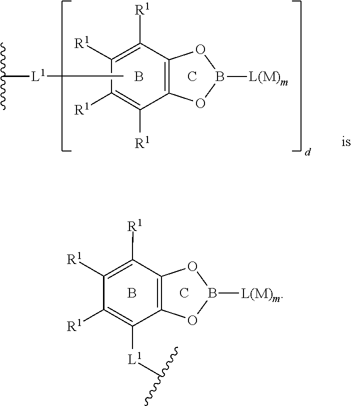

In some embodiments,

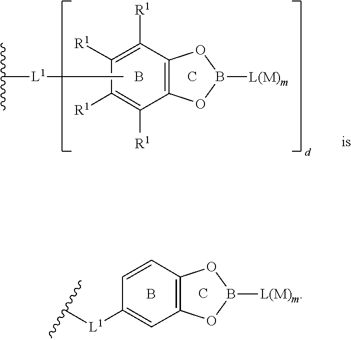

In certain embodiments,

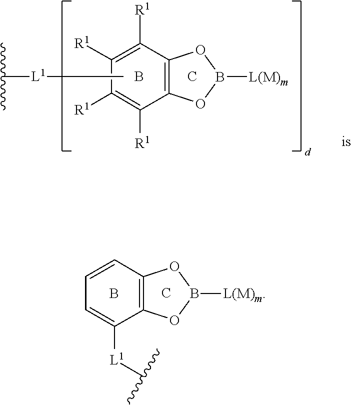

In certain embodiments, each instance of R^{a} is hydrogen. In certain embodiments, at least one instance of R^{a} is hydrogen. In certain embodiments, each instance of R^{a} is halogen. In certain embodiments, at least one instance of R^{a} is halogen. In certain embodiments, each instance of R^{a} is unsubstituted C₁₋₆ alkyl. In certain embodiments, at least one instance of R^{a} is unsubstituted C₁₋₆ alkyl. In certain embodiments, each instance of R^{a} is substituted C₁₋₆ alkyl. In certain embodiments, at least one instance of R^{a} is substituted C₁₋₆ alkyl. In certain embodiments, each instance of R^{a} is substituted methyl. In certain embodiments, at least one instance of R^{a} is substituted methyl (e.g., CF₃, CHF₂, CH₂F). In some embodiments, R^{a} is substituted or unsubstituted, C₂₋₆ alkenyl or substituted or unsubstituted, C₂₋₆ alkynyl. In certain embodiments, at least one instance of R^{a} is substituted or unsubstituted carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R^{a} is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R^{a} is substituted or unsubstituted heterocyclyl (e.g., substituted or unsubstituted, 3- to 7-membered, monocyclic heterocyclyl). In certain embodiments, at least one instance of R^{a} is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R^{a} is substituted or unsubstituted aryl. In certain embodiments, at least one instance of R^{a} is substituted or unsubstituted phenyl. In certain embodiments, at least one instance of R^{a} is substituted or unsubstituted naphthyl. In certain embodiments, at least one instance of R^{a} is substituted or unsubstituted heteroaryl. In certain embodiments, at least one instance of R^{a} is substituted or unsubstituted, 5- to 6-membered, monocyclic heteroaryl. In certain embodiments, at least one instance of R^{a} is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R^{a} is substituted or unsubstituted pyridinyl, substituted or unsubstituted pyrazinyl, substituted or unsubstituted pyrimidinyl, or substituted or unsubstituted pyridazinyl. In certain embodiments, at least one instance of R^{a} is substituted or unsubstituted, 9- to 10-membered, bicyclic heteroaryl. In certain embodiments, at least one instance of R^{a} is a nitrogen protecting group (e.g., Bn, Boc, Cbz, Fmoc, trifluoroacetyl, triphenylmethyl, acetyl, or Ts) when attached to a nitrogen atom. In certain embodiments, at least one instance of R^{a} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, two instances of R^{a} are joined to form substituted or unsubstituted heterocyclyl (e.g., substituted or unsubstituted, 3- to 7-membered, monocyclic heterocyclyl). In certain embodiments, two instances of R^{a} are joined to form substituted or unsubstituted heteroaryl (e.g., substituted or unsubstituted, 5- to 6-membered, monocyclic heteroaryl).

In certain embodiments, each L is the same. In certain embodiments, each L is different. In certain embodiments, some instances of L are the same and some instances of L are different. In some embodiments, each instance of L is a bond. In certain embodiments, some instances of L are a bond.

In certain embodiments, at least one instance of L is substituted or unsubstituted, C₂₋₃₀₀ alkenylene, substituted or unsubstituted, C₂₋₃₀₀ alkynylene, substituted or unsubstituted, C₂₋₃₀₀ heteroalkenylene, or C₂₋₃₀₀ heteroalkynylene wherein optionally one or more backbone carbon atoms in each instance of substituted or unsubstituted, C₂₋₃₀₀ alkylene, substituted or unsubstituted, C₂₋₃₀₀ alkenylene, substituted or unsubstituted, C₂₋₃₀₀ alkynylene, substituted or unsubstituted, C₂₋₃₀₀ heteroalkylene, substituted or unsubstituted, C₂₋₃₀₀ heteroalkenylene, or C₂₋₃₀₀ heteroalkynylene are independently replaced with a substituted or unsubstituted carbocyclene, substituted or unsubstituted heterocyclene, substituted or unsubstituted arylene, or substituted or unsubstituted heteroarylene.

In certain embodiments, at least one instance of L is substituted or unsubstituted, C₂₋₃₀ alkylene wherein optionally one or more backbone carbon atoms are independently replaced with a substituted or unsubstituted carbocyclene, substituted or unsubstituted heterocyclene, substituted or unsubstituted arylene, or substituted or unsubstituted heteroarylene. In certain embodiments, at least one instance of L is substituted or unsubstituted, C₂₋₃₀₀ alkylene wherein optionally one or more backbone carbon atoms are independently replaced with a substituted or unsubstituted arylene or substituted or unsubstituted heteroarylene. In certain embodiments, at least one instance of L is substituted or unsubstituted, C₂₋₃₀₀ alkylene wherein optionally one or more backbone carbon atoms are independently replaced with a substituted or unsubstituted arylene or substituted or unsubstituted heteroarylene.

In some embodiments, at least one instance of L is substituted or unsubstituted, C₂₋₃₀₀ heteroalkylene wherein optionally one or more backbone carbon atoms and/or heteroatoms are independently replaced with a substituted or unsubstituted carbocyclene, substituted or unsubstituted heterocyclene, substituted or unsubstituted arylene, or substituted or unsubstituted heteroarylene. In some embodiments, at least one instance of L is substituted or unsubstituted, C₂₋₃₀₀ heteroalkylene wherein optionally one or more backbone carbon atoms and/or heteroatoms are independently replaced with a substituted or unsubstituted arylene or substituted or unsubstituted heteroarylene. In some embodiments, at least one instance of L is substituted or unsubstituted, C₂₋₃₀₀ heteroalkylene wherein optionally one or more backbone carbon atoms and/or heteroatoms are independently replaced with a substituted or unsubstituted arylene or substituted or unsubstituted heteroarylene.

In certain embodiments, at least one instance of L comprises in the backbone thereof a polymer. In some embodiments, at least one instance of the polymer is a polyethylene glycol (PEG), a polyethylene oxide (PEO), a polypropylene glycol (PPG), a polyglycerol (PG), a poloxamine (POX), a polybutylene oxide (PBO), polylactic acid (PLA), polyglycolic acid (PGA), poly(lactic-co-glycolic acid) (PLGA), polycaprolactone (PCL), polydioxanone (PDO), a polyanhydride, a polyacrylide, a polyvinyl, or a polyorthoester. In some embodiments, at least one instance of the polymer is polyethylene glycol (PEG). In certain embodiments, the weight-average molecular weight of at least one instance of the polymer is between 200 and 500, between 500 and 1,000, between 1,000 and 2,000, between 2,000 and 5,000, between 5,000 and 10,000, or between 10,000 and 50,000, inclusive, g/mol. In some embodiments, the weight-average molecular weight of at least one instance of the polymer is between 1,000 and 5,000, inclusive, g/mol. In certain embodiments, the weight-average molecular weight of at least one instance of the polymer is between 2,000 and 5,000 inclusive, g/mol.

In certain embodiments, the agent is covalently bound to the polymer chain, through a cleavable linker (which can also be referred to herein as a “sensitive linker”). In certain embodiments, at least one instance (e.g., each instance) of L comprises a cleavable linker. In certain embodiments, at least one instance (e.g., each instance) of L is a cleavable linker. A cleavable linker is “cleaved” or “degraded” when one or more bonds of the cleavable linker are broken, e.g., resulting in release of an agent, e.g., from the conjugate or particle. Linker cleavage or agent release need not be 100%, e.g., a cleavage or release of at least 10%, 20%, 30%, 40%, 50%, 60%, 70%, 80%, 90%, 95%, or higher, e.g., over a period of seconds, minutes, hours (e.g., 6 hours, 12 hours, or 24 hours), days (e.g., 2 days or 7 days), weeks, or months is encompassed by this term.

In some embodiments, the cleavable linker is cleavable by or is sensitive to an enzyme (e.g., an esterase or a protease), pH (e.g., acidic pH, basic pH), light (e.g., ultraviolet light), a nucleophile, reduction, or oxidation. In some embodiments, the cleavable linker is cleavable by or is sensitive to an enzyme (e.g., an esterase or a protease) or pH (e.g., acidic pH, basic pH). In some embodiments, the cleavable linker is not cleavable by light (e.g., ultraviolet light).

In some embodiments, the cleavable linker comprises an ester, an acetal, a ketal, a phosphoramidite, a hydrazone, an imine, an oxime, a disulfide, or a silyl moiety, a combination of acetal or ketal with ester group, an oligo-acetal or oligo-ketal group, a combination of the oligo-ketal and silyl ether group, or a combination of the oligo-ketal and vinyl ether group. In some embodiments, the cleavable linker comprises an ester. In some embodiments, the cleavable linker comprises an acetal. In some embodiments, the cleavable linker comprises a phosphoramidite. In some embodiments, the cleavable linker comprises a hydrazine. In some embodiments, the cleavable linker comprises an imine. In some embodiments, the cleavable linker comprises an oxime. In some embodiments, the cleavable linker comprises a silyl moiety. In some embodiments, the cleavable linker comprises a disulfide.

In other embodiments, the cleavable linker is chosen from a combination of acetal or ketal with cis-aconityl, hydrazine, oxime, imidazole, or trityl groups. Any of the aforesaid groups or combination of groups can modified to enhance the pH sensitivity of the cleavable linker, e.g., as described herein.

In some embodiments, the cleavable linker is an amide, urea, carbamate, carbonate, or disulfide.

The cleavable linker may include an atom or a part of a moiety that is derived in part from the agent (e.g., a therapeutic agent).

In some embodiments, the cleavable linker is cleaved or degraded, e.g., preferentially cleaved or degraded, upon exposure to a first set of conditions relative to a second set of conditions. For example, the cleavable linker can be “preferentially cleaved” or “preferentially degraded” in a first set of conditions relative to a second set of conditions if at least 10%, 20%, 30%, 40%, 50%, 60%, 70%, 80%, 90%, 95%, or more of a bond or bonds of the cleavable linker are broken, or the agent is released, in the first set of conditions relative to the second set of conditions.

In some embodiments, the cleavable linker is degraded or hydrolyzed at physiological conditions. In some embodiments, the linker is pH sensitive or cleaved at a certain pH. In some embodiments, the linker is degraded or hydrolyzed through the action of an enzyme (e.g., a protease or esterase). For example, in some embodiments, the cleavable linker is preferentially cleaved in a tissue microenvironment, e.g., a tumor microenvironment, which is referred to herein as a “tissue microenvironment cleavable linker.” In embodiments, the tissue (e.g., tumor) microenvironment cleavable linker is preferentially cleaved or degraded upon exposure to a first desired tissue or tumor microenvironment relative to a second tissue or non-tumor tissue. A tissue (e.g., tumor) microenvironment cleavable linker can be preferentially cleaved if at least 10%, 20%, 30%, 40%, 50%, 60%, 70%, 80%, 90%, 95% or more of a bond or bonds of the linker are broken, or the agent is released, in a desired tissue or tumor microenvironment relative to another tissue or non-tumor tissue. In one embodiments, the tissue (e.g., tumor) microenvironment cleavable linker is preferentially cleaved or degraded if one or more of the bonds of the linker are broken, or the agent is released, at least 2, 5, 10, 20, 30, 40, 50, 60, 70, 80, or 100 times faster upon exposure to a first desired tissue or tumor microenvironment relative to a second tissue or non-tumor tissue. The tissue (e.g., tumor) microenvironment can have a particular set of conditions, e.g., pH, enzymes, that cause the cleavage or degradation of the linker.

In some embodiments, the cleavable linker is a peptide. In some embodiments, the linker is a peptide, and the peptide sequence is comprised of naturally occurring amino acids. In some embodiments, the linker is a peptide, and the peptide sequence comprises at least one synthetically derived amino acids, e.g., at least 2, at least 3, at least 4, at least 5, at least 8, at least 10, at least 15, at least 20, or more synthetically derived amino acids (unnatural amino acid). In some embodiments, the peptide has a linear structure. In some embodiments, the peptide has a branched structure. In some embodiments, the peptide has a branched structure with, e.g., at least 2, at least 3, at least 4, at least 5, at least 6, at least 7, or at least 8 branching points. In some embodiments, the peptide has a cyclic structure.

In some embodiments, the cleavable linker is a peptide, and the peptide sequence comprises at least 2 amino acid residues. In some embodiments, the peptide sequence comprises at least 3, at least 4, at least 5, at least 6, at least 7, at least 8, at least 9, or at least 10 amino acid residues. In some embodiments, the peptide sequence is from about 1 to about 10 amino acid residues. In some embodiments, the peptide sequence is from about 1 to about 15, about 20, about 25, about 30, about 40, about 50, about 60, about 70, about 80, about 90, or about 100 amino acid residues. In some embodiments, the peptide sequence is from about 10 to about 100 amino acid residues. In some embodiments, the peptide sequence is from about 25 to about 100 amino acid residues. In some embodiments, the peptide sequence is from about 50 to about 100 amino acid residues.

In some embodiments, the cleavable linker comprises a substrate peptide that is cleaved, e.g., activated, by a matrix metalloprotease (MMP) selected from a sequence disclosed in U.S. Patent Application No. 2015/0087810 with a publication date of Mar. 26, 2015. In some embodiments, the substrate peptide comprises a protease substrate comprising an amino acid sequence selected from the group consisting of SEQ ID NOs: 353-363, 372-375, 376-378, 395-401, 411-419, 426-433, 437-449, 454-456, 459-469, 475-482, 487-495, 318-323.325-327.330-335, 341-347, 14-33, and 159, e.g., as described in U.S. Patent Application No. 2015/0087810. In some embodiments, the linker comprises a substrate peptide derived from a sequence disclosed in U.S. Pat. No. 8,541,203, e.g., a substrate peptide chosen from an enzyme selected from the group consisting of MMP-1, MMP-2, MMP-3, MMP-8, MMP-9, MMP-14, plasmin, PSA, PSMA, CATHEPSIN D. CATHEPSIN K, CATHEPSIN S. ADAM10, ADAM12, ADAMTS, Caspase-1, Caspase-2, Caspase-3, Caspase-4, Caspase-5, Caspase-6, Caspase-7. Caspase-8, Caspase-9, Caspase-10, Caspase-11, Caspase-12, Caspase-13, Caspase-14, and TACE. In some embodiments, the linker comprises a sequence disclosed in U.S. Pat. No. 8,513,390. In some embodiments, the linker comprises a sequence disclosed in International Patent Publication No. WO2003/079972. In some embodiments, the linker comprises a sequence disclosed in U.S. Pat. No. 7,495,099. In some embodiments, the linker comprises a sequence disclosed in U.S. Pat. No. 8,580,244. In some embodiments, the linker comprises a sequence disclosed in one of the following articles: van Kempen, et al., Eur Cancer (2006) 42:728-734; Desnoyers, L. R, et al., Sci Transl Med (2013) 5:207ra144; Rice. J. J, et al., Protein Sci (2006) 15:825-836; Boulware, K. T, and Daugherty, P. S. Proc Natl Acad Sci USA (2006) 103:7583-7588; Deperthes, D. Biol Chem (2002) 383:1107-1112; Harris, J. L. Proc Natl Acad Sci USA (2000) 97:7754-7759; Salmaso S, and Caliceti, P. J Drug Deliv (2013) 2013:1-19; and Eckhard, U et al., Matrix Biol (2015) doi: 10.1016/j.matbio.2015.09.003 (epub ahead of print). The contents of any of the publications referenced herein are hereby expressly incorporated by reference.

In some embodiments, the cleavable linker comprises a substrate peptide that is cleaved, e.g., activated, by a protease, e.g., a protease present in a tumor or fibrotic microenvironment (e.g., a matrix metalloprotease (MMP), e.g., as described by Desnoyers, L. R, et al., Sci Transl Med (2013) 5:207ra144; Eckhard, U et al Matrix Biol (2015) doi: 10.1016/j.matbio.2015.09.003 (epub ahead of print); and van Kempen, et al., Eur Cancer (2006) 42:728-734. In one embodiments, the linker includes the amino acid sequence of a substrate for uPA, e.g., comprises the amino acid sequence LSGRSDNH (SEQ ID NO:1), e.g., as described in U.S. Pat. No. 8,513,390. In some embodiments, the linker sequence further includes a Gly-Ser-containing peptide linker, at either end, or both ends to the substrate peptide. Additional exemplary proteases that may be upregulated in a tumor microenvironment include, but are not limited to, urokinase-type plasminogen activator (uPA), which is upregulated in human carcinomas (S. Ulisse, et al., Curr. Cancer Drug Targets 9, 32-71 (2009)), membrane-type serine protease 1 (MT-SP/matriptase) (K. Uhland Cell. Mol. Life Sci. 63, 2968-2978 (2006): A. M. LeBeau, et al., Proc. Natl. Acad. Sci. U.S.A, 110, 93-98 (2013)), and legumain, a lysosomal protease found to be released and active in the acidic extracellular tumor microenvironment (C. Liu, et al., Cancer Res, 63, 2957-2964 (2003)). In some embodiments, the protease is produced by an inflammatory cell, e.g., a tumor infiltrating leukocyte (e.g., a leukocyte-derived MMP), e.g., as described by van Kempen, et al., Eur Cancer (2006) 42:728-734. In other embodiments, the MMP is chosen from MMP1, MMP2, MMP3, MMP7, MMP8, MMP9, MMP12, MMP13 or MMP14, e.g., as described by Eckhard. U et al., supra.

In some embodiments, the substrate peptide is derived from a CLiPS library (as described in, e.g., K. T. Boulware, P. S. Daugherty, Proc. Natl. Acad. Sci. U.S.A, 103, 7583-7588 (2006)). In other embodiments, the substrate peptide specificity is evaluated using combinatorial fluorogenic substrate libraries, e.g., as described by Harris, J. L. Proc Natl Acad Sci USA (2000) 97:7754-7759. In other embodiments, the substrate peptide is derived from a phage display library (e.g., it is a phase display substrate), e.g., as described by Deperthes, D. Biol Chem (2002) 383:1107-1112. For example, a phage display substrate is exposed to a plurality of proteases; peptides released through specific cleavage can be amplified in an expression system. In other embodiments, the substrate peptide is derived from a bacterial display library, e.g., as described by Rice, J. J, et al., Protein Sci (2006) 15:825-836.

In one embodiments, the tissue (e.g., tumor) microenvironment cleavable linker is cleavable by an enzyme. In some embodiments, the enzyme comprises an esterase or a protease. Exemplary proteases include MMP-1, MMP-2, MMP-3, MMP-8, MMP-9, MMP-14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, or TACE.

In other embodiments, the tissue microenvironment cleavable linker is cleavable at a particular pH. In some embodiments, the tissue microenvironment cleavable linker is cleavable at a pH between about 5.0 and about 7.4, between 5.0 and 7.0, between 5.0 and 6.5, between 5.0 and 5.5, or between 5.9 and 6.2. In one embodiment, the tissue microenvironment cleavable linker is cleavable at a pH between about 6.0 and about 7.0, between about 6.2 and about 6.9, between about 6.5 and about 6.8, or between about 6.5 and about 6.7. In one embodiment, the tissue microenvironment cleavable linker is cleavable at a pH between about 5.5 and about 6.5, e.g., between 5.9 and 6.2. In one embodiment, the tissue microenvironment cleavable linker is cleavable at a hypoxic pH, e.g., a pH about 6.7 to 6.9, e.g., compared to a physiological pH of about 7.4.

In some embodiments, the tissue microenvironment cleavable linker is cleavable is cleaved at a pH of no more than 7.4, no more than 7.0, no more than 6.9, no more than 6.8, no more than 6.7, no more than 6.6, no more than 6.5, no more than 6.4, no more than 6.3, no more than 6.2, no more than 6.1, no more than 6.0, no more than 5.5 or lower.

In one embodiment, the tissue microenvironment cleavable linker is preferentially cleaved or degraded upon exposure to a first pH relative to a second pH. In one embodiment, the tissue microenvironment cleavable linker is cleaved or degraded at least 2, 5, 10, 20, 30, 40, 50, 60, 70, 80, or 100 times faster upon exposure to a first pH relative to a second pH. In other embodiments, the tissue microenvironment cleavable linker shows a greater release or degradation rate at a first acidic pH (e.g., pH=6.7) relative to a second more basic pH (e.g., pH=7.4). In one embodiment, ratio of release or degradation rate of the tissue microenvironment cleavable linker at pH=6.7 relative to pH=7.4 is greater than 1, 1.2, 1.4, 1.6, 1.8, 2, 2.2, 2.4, 2.6, 2.8, 3 or higher. In one embodiment, ratio of release or degradation rate of the tissue microenvironment cleavable linker at pH=6.7 relative to pH=7.4 is greater than 2.

In one embodiment, the tissue microenvironment cleavable linker shows increased pH-sensitivity in a hypoxic microenvironment, e.g., in a tumor, or fibrotic tissue.

In some embodiments, the tissue microenvironment cleavable linker exhibits an increased release rate or increased release yield of the agent at a desired site (e.g., a tumor), e.g., relative to the release rate or release yield at another site. In one embodiment, the tissue microenvironment cleavable linker comprises an electron withdrawing group (e.g., an electron withdrawing group that enhances the cleavage rate or yield.

In certain embodiments, M is an agent. In certain embodiments, at least one instance of M is a pharmaceutical agent. In certain embodiments, all instances of M are a pharmaceutical agent. An agent can be a molecule, group of molecules, complex or substance administered to an organism for diagnostic, therapeutic, preventative medical, or veterinary purposes. In certain embodiments, the agent is a pharmaceutical agent. In certain embodiments the pharmaceutical agent is a therapeutic agent, a diagnostic agent, or a prophylactic agent.

In certain embodiments, the therapeutic agent is an immunomodulatory agent. In certain embodiments, the therapeutic agent is an immunosuppressant. In certain embodiments, the therapeutic agent is an immunoactivator. In certain embodiments, the therapeutic agent is an interleukin (e.g., IL-2, IL-7, IL-12). In certain embodiments, the therapeutic agent is a cytokine (e.g., interferon, G-CSF). In certain embodiments, the therapeutic agent is a chemokine (e.g., CCL3, CCL26, CXCL7). In certain embodiments, the therapeutic agent is an immunomodulatory imide drug. In certain embodiments, the therapeutic agent is thalidomide, lenalidomide, or pomalidomide. In certain embodiments, the therapeutic agent is cytosine phosphate-guanosine, oligodeoxynucleotide, or glucan.

In certain embodiments, the therapeutic agent is an immune checkpoint inhibitor.

An immune checkpoint inhibitor, as used herein, is an agent that inhibits or prevents the activity of an immune checkpoint molecule, e.g., by binding to the molecule. An immune checkpoint inhibitor may reduce the immune checkpoint molecule activity in a cell or organism, e.g., by at least 5%, at least 10%, at least 20%, at least 30%, at least 40%, at least 50%, at least 60%, at least 70%, at least 80%, at least 90%, or about 100%, compared to a cell or organism that has not been exposed to the immune checkpoint inhibitor. Immune checkpoint molecule activity may be interfered with by antibodies that bind selectively to and block the activity of the immune checkpoint molecule. The activity of the immune checkpoint molecule can also be inhibited or blocked by molecules other than antibodies, such as proteins, small molecules, and peptides that bind to the immune checkpoint molecule. Agents that bind to and degrade or inhibit the DNA or mRNA encoding the immune checkpoint molecule also can act an immune checkpoint inhibitor. Examples include siRNAs and antisense oligonucleotides. Non-limiting example immune checkpoint molecules include programmed cell death 1 protein (PD-1), programmed cell death 1 protein ligand 1 (PD-L1), cytotoxic T-lymphocyte-associated protein 4 (CTLA-4), T-cell immunoglobulin domain and mucin domain 3 (TIM3), lymphocyte activation gene-3 (LAG3). V-set domain-containing T-cell activation inhibitor 1 (VTCN₁ or B7-H4), cluster of differentiation 276 (CD276 or B7-H3). B and T lymphocyte attenuator (BTLA), galectin-9 (GAL9), checkpoint kinase 1 (Chk1), adenosine A2A receptor (A2AR), indoleamine 2,3-dioxygenase (IDO), killer-cell immunoglobulin-like receptor (KIR), and V-domain Ig suppressor of T cell activation (VISTA).

In some embodiments, the immune checkpoint inhibitor is an antibody, such a humanized or human antibody. As used herein, the term “antibody” refers to an immunoglobulin molecule that specifically binds to a particular antigen such as an immune checkpoint molecule (e.g., PD-L1, PD-1, or CTLA-4) and includes polyclonal, monoclonal, genetically engineered and otherwise modified forms of antibodies, including but not limited to chimeric antibodies, humanized antibodies, fully human antibodies, heteroconjugate antibodies (e.g., bispecific antibodies, diabodies, triabodies, and tetrabodies), and antigen binding fragments of antibodies, including e.g., Fab′, F(ab′)₂, Fab. Fv, rlgG, and scFv fragments. Moreover, unless otherwise indicated, the term “monoclonal antibody” is meant to include both intact molecules, as well as, antibody fragments (such as, for example. Fab and F(ab′)₂ fragments) which are capable of specifically binding to the antigen. An antibody may include an immunoglobulin constant domain from any immunoglobulin, such as IgG1, IgG2, IgG3, or IgG4 subtypes. IgA (including IgA1 and IgA2), IgE, IgD or IgM.

In some embodiments, the immune checkpoint inhibitor is an antibody (e.g., a monoclonal antibody such as a human or humanized monoclonal antibody) to an immune checkpoint molecule, such as programmed cell death 1 protein (PD-1), programmed cell death 1 protein ligand 1 (PD-L1), cytotoxic T-lymphocyte-associated protein 4 (CTLA-4), T-cell immunoglobulin domain and mucin domain 3 (TIM3), lymphocyte activation gene-3 (LAG3), V-set domain-containing T-cell activation inhibitor 1 (VTCN₁ or B7-H4), cluster of differentiation 276 (CD276 or B7-H3), B and T lymphocyte attenuator (BTLA), galectin-9 (GAL9), checkpoint kinase 1 (Chk1), adenosine A2A receptor (A2AR), indoleamine 2,3-dioxygenase (IDO), killer-cell immunoglobulin-like receptor (KIR), or V-domain Ig suppressor of T cell activation (VISTA).

In some embodiments, immune checkpoint inhibitor is a small molecule, wherein the molecular weight of the small molecule is not more than 1.500 g/mol.

In some embodiments, the immune checkpoint inhibitor is a Programmed Cell Death Ligand 1 (PD-L1) or Programmed Cell Death 1 (PD-1) inhibitor.

A PD-1 inhibitor, as used herein is an agent that inhibits or prevents PD-1 activity, e.g., by binding to PD-1. A PD-1 inhibitor may reduce PD-1 activity in a cell or organism, e.g., by at least 5%, at least 10%, at least 20%, at least 30%, at least 40%, at least 50%, at least 60%, at least 70%, at least 80%, at least 90%, or about 100%, compared to a cell or organism that has not been exposed to the PD-1 inhibitor. Human PD-1 is encoded by the gene PDCD1 (Genbank Entrez ID 5133). PD-1 functions as an immune checkpoint and negatively regulates immune responses, e.g. inhibiting the activation, expansion, and/or function of CD8⁺ T-cells and other immune cells. PD-L1 is a ligand for PD-1. PD-L1 is a type 1 transmembrane protein with immunoglobulin V-like and C-like domains. Human PD-L1 is encoded by the CD274 gene (Genbank Entrez ID 29126). PD-L1 is also a ligand for B7.1.

PD-1 activity may be interfered with by antibodies that bind selectively to and block the activity of PD-1. The activity of PD-1 can also be inhibited or blocked by molecules other than antibodies, such as proteins, small molecules, and peptides, that bind PD-1. Agents that bind to and degrade or inhibit the DNA or mRNA encoding PD-1 also can act as PD-1 inhibitor. Examples include anti-PD-1 siRNAs and anti-PD-1 antisense oligonucleotides.

A PD-L1 inhibitor, as used herein is an agent that inhibits or prevents PD-L1 activity, e.g., by binding to PD-L1. A PD-L1 inhibitor may reduce PD-L1 activity in a cell or organism, e.g., by at least 5%, at least 10%, at least 20%, at least 30%, at least 40%, at least 50%, at least 60%, at least 70%, at least 80%, at least 90%, or about 100%, compared to a cell or organism that has not been exposed to the PD-L inhibitor.

PD-L1 activity may be blocked by molecules that selectively bind to and block the activity of PD-L1, e.g. by blocking the interaction with and activation of PD-1 and/or B7-1. The activity of PD-L1 can also be inhibited or blocked by molecules other than antibodies, such as proteins, small molecules, and peptides, that bind PD-L. Agents that bind to and degrade or inhibit the DNA or mRNA encoding PD-L1 also can act as PD-L1 inhibitors. Examples include anti-PD-L1 siRNAs and anti-PD-L1 antisense oligonucleotides.

Example PD-1 inhibitors include those described in U.S. Publications 20130280265, 20130237580, 20130230514.20130109843, 20130108651.20130017199, 20120251537, and 20110271358, and in European Patent EP2170959B1, the entire disclosures of which are incorporated herein by reference.

Example PD-1 inhibitors include: nivolumab (e.g., OPDIVO® from Bristol-Myers Squibb), a fully human IgG4 monoclonal antibody that binds PD-1; pidilizumab (e.g., CT-011 from CureTech), a humanized IgG1 monoclonal antibody that binds PD-1; pembrolizumab (e.g., KEYTRUDA® from Merck), a humanized IgG4-kappa monoclonal antibody that binds PD-1; MEDI-0680 (AstraZeneca/MedImmune) a monoclonal antibody that binds PD-1; and REGN2810 (Regeneron/Sanofi) a monoclonal antibody that binds PD-1. Another exemplary PD-1 inhibitor is AMP-224 (Glaxo Smith Kline and Amplimmune), a recombinant fusion protein composed of the extracellular domain of the Programmed Cell Death Ligand 2 (PD-L2) and the Fc region of human IgG1, that binds to PD-1.

Example PD-L1 inhibitors include those described in U.S. Publications 20090055944, 20100203056, 20120039906, 20130045202, 20130309250, and 20160108123, the entire disclosures of which are incorporated herein by reference.

Example PD-L1 inhibitors include, for example: atezolizumab (also called TECENTRIQ™, Genentech/Roche), an human monoclonal antibody that binds to PD-L1; durvalumab (also called MED14736, AstraZeneca/MedImmune), a human immunoglobulin IgG1 kappa monoclonal antibody that binds to PD-L; BMS-936559 (Bristol-Meyers Squibb), a fully human IgG4 monoclonal antibody that binds to PD-L; avelumab (also called MSB 0010718C, Merck KGaA/Pfizer), a fully human IgG1 monoclonal antibody that binds to PD-L1; and CA-170 (Aurigene/Curis) a small molecule antagonist of PD-L1.

In some embodiments, the immune checkpoint inhibitor is a cytotoxic T-lymphocyte-associated protein 4 (CTLA-4) inhibitor.

A CTLA-4 inhibitor, as used herein is an agent that inhibits or prevents CTLA-4 activity, e.g., by binding to CTLA-4. A CTLA-4 inhibitor may reduce CTLA-4 activity in a cell or organism, e.g., by at least 5%, at least 10%, at least 20%, at least 30%, at least 40%, at least 50%, at least 60%, at least 70%, at least 80%, at least 90%, or about 100%, compared to a cell or organism that has not been exposed to the CTLA-4 inhibitor. Human CTLA-4 is encoded by the gene CTLA4 (Genbank Entrez ID 1493). CTLA-4 negatively regulates immune responses, e.g. by transmitting inhibitory signals to T cells.

CTLA-4 activity may be interfered with by antibodies that bind selectively to and block the activity of CTLA-4. The activity of CTLA-4 can also be inhibited or blocked by molecules other than antibodies, such as proteins, small molecules, and peptides, that bind CTLA-4. Agents that bind to and degrade or inhibit the DNA or mRNA encoding CTLA-4 also can act as CTLA-4 antagonists. Examples include anti-CTLA-4 siRNAs and anti-CTLA-4 antisense oligonucleotides.

Example CTLA-4 antagonists include those described in PCT Publication Nos. WO2001/014424. WO2012/118750, European Patent No. EP1212422B1, U.S. Pat. Nos. 5,811,097, 5,855,887, 6,051,227, 6,984,720, 7,034,121, 7,824,679, 8,017,114, 8,475,790, 8,318,916, 8,685,394. U.S. Publication Nos, 2002/0039581, 2005/0201994, and 2009/0117037, the entire disclosures of which are incorporated herein by reference.

Example CTLA-4 antagonists include: ipilimumab (YERVOY®, Bristol-Myers Squibb), which is a recombinant human IgG1 monoclonal antibody against CTLA-4, and tremelimumab (AstraZeneca; MedImmune/Pfizer), which is a human IgG2 monoclonal antibody against CTLA-4.

In some embodiments, the immune checkpoint inhibitor is a T-cell immunoglobulin domain and mucin domain 3 (TIM3) inhibitor, lymphocyte activation gene-3 (LAG3) inhibitor, V-set domain-containing T-cell activation inhibitor 1 (VTCN₁ or B7-H4) inhibitor, cluster of differentiation 276 (CD276 or B7-H3) inhibitor, B and T lymphocyte attenuator (BTLA) inhibitor, galectin-9 (GAL9) inhibitor, checkpoint kinase 1 (Chk1) inhibitor, adenosine A2A receptor (A2AR) inhibitor, indoleamine 2,3-dioxygenase (IDO) inhibitor, killer-cell immunoglobulin-like receptor (KIR) inhibitor, or V-domain Ig suppressor of T cell activation (VISTA) inhibitor.

In certain embodiments, the therapeutic agent is an anti-cancer agent. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF), and antibodies (e.g. HERCEPTIN (trastuzumab). T-DM1. AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent) docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonucleotide reductase inhibitors (e.g. hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g. 1-methyl-4-phenylpyridinium ion), cell cycle inhibitors (e.g., staurosporine), actinomycin (e.g. actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone). MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC₂₂₀, dovitinib lactate (TKI258. CHIR-258), BIBW 2992 (TOVOK®), SGX523. PF-04217903, PF-02341066, PF-299804. BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus. AP23573 (Ariad), AZD8055 (AstraZeneca), BEZ235 (Novartis). BGT226 (Norvartis), XL765 (Sanofi Aventis), PF-4691502 (Pfizer), GDC0980 (Genetech), SF1126 (Semafoe), and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In certain embodiments, the anti-cancer agent is selected from the group consisting of abiraterone acetate, ABVD, ABVE, ABVE-PC, AC, AC-T, ADE, ado-trastuzumab emtansine, afatinib dimaleate, aldesleukin, alemtuzumab, anastrozole, arsenic trioxide, asparaginase Erwinia chrysanthemi, axitinib, azacitidine. BEACOPP, belinostat, bendamustine hydrochloride, BEP, bevacizumab, bicalutamide, bleomycin, blinatumomab, bortezomib, bosutinib, brentuximab vedotin, busulfan, cabazitaxel, cabozantinib-s-malate, CAF, capecitabine, CAPOX, carboplatin, carboplatin-taxol, carfilzomibcarmustine, carmustine ceritinib, cetuximab, chlorambucil, chlorambucil-prednisone, CHOP, cisplatin, clofarabine, COPP, COPP-ABV, crizotinib. CVP, cyclophosphamide, cytarabine, dabrafenib, dacarbazine, dactinomycin, dasatinib, daunorubicin hydrochloride, decitabine, degarelix, denileukin diftitox, denosumab. Dinutuximab, docetaxel, doxorubicin hydrochloride, doxorubicin hydrochloride liposome, enzalutamide, epirubicin hydrochloride, EPOCH, erlotinib hydrochloride, etoposide, etoposide phosphate, everolimus, exemestane, FEC, fludarabine phosphate, fluorouracil, FOLFIRI, FOLFIRI-BEVACIZUMAB, FOLFIRI-CETUXIMAB, FOLFIRINOX, FOLFOX, FU-LV, fulvestrant, gefitinib, gemcitabine hydrochloride, gemcitabine-cisplatin, gemcitabine-oxaliplatin, goserelin acetate, Hyper-CVAD, ibritumomab tiuxetan, ibrutinib, ICE, idelalisib, ifosfamide, imatinib mesylate, imiquimod, ipilimumab, irinotecan hydrochloride, ixabepilone, lanreotide acetate, lapatinib ditosylate, lenalidomide, lenvatinib, letrozole, leucovorin calcium, leuprolide acetate, liposomal cytarabine, lomustine, mechlorethamine hydrochloride, megestrol acetate, mercaptopurine, methotrexate, mitomycin c, mitoxantrone hydrochloride, MOPP, nelarabine, nilotinib, nivolumab, obinutuzumab, OEPA, ofatumumab, OFF, olaparib, omacetaxine mepesuccinate, OPPA, oxaliplatin, paclitaxel, paclitaxel albumin-stabilized nanoparticle formulation. PAD, palbociclib, pamidronate disodium, panitumumab, panobinostat, pazopanib hydrochloride, pegaspargase, peginterferon alfa-2b, peginterferon alfa-2b, pembrolizumab, pemetrexed disodium, pertuzumab, plerixafor, pomalidomide, ponatinib hydrochloride, pralatrexate, prednisone, procarbazine hydrochloride, radium 223 dichloride, raloxifene hydrochloride, ramucirumab, R-CHOP, recombinant HPV bivalent vaccine, recombinant human papillomavirus, nonavalent vaccine, recombinant human papillomavirus, quadrivalent vaccine, recombinant interferon alfa-2b, regorafenib, rituximab, romidepsin, ruxolitinib phosphate, siltuximab, sipuleucel-t, sorafenib tosylate. STANFORD V, sunitinib malate, TAC, tamoxifen citrate, temozolomide, temsirolimus, thalidomide, thiotepa, topotecan hydrochloride, toremifene, tositumomab and iodine 1131, tositumomab, TPF, trametinib, trastuzumab, VAMP, vandetanib, VEIP, vemurafenib, vinblastine sulfate, vincristine sulfate, vincristine sulfate liposome, vinorelbine tartrate, vismodegib, vorinostat, XELIRI, XELOX, ziv-aflibercept, and zoledronic acid. In some embodiments, the anti-cancer agent is bortezomib.

In certain embodiments, the agent is an anti-hypertension agent. Exemplary anti-hypertension agents include, but are not limited to, amiloride, amlodipine, atenolol, azilsartan, benazepril, bendroflumethiazide, betaxolol, bisoprolol, bucindolol, bumetanide, candesartan, captopril, carteolol, carvedilol, chlorothiazide, chlorthalidone, cilnidipine, clevidipine, diltiazem, doxazosin, enalapril, epitizide, eplerenone, eprosartan, ethacrynic acid, felodipine, Fimasartan, fosinopril, furosemide, hydrochlorothiazide, indapamide, indoramin, irbesartan, isradipine, labetalol, lercanidipine, levamlodipine, lisinopril, losartan, methyclothiazide, metolazone, metoprolol, moexipril, nadolol, nebivolol, nicardipine, nifedipinc, nimodipine, nisoldipine, nitrendipine, olmesartan, oxprenolol, penbutolol, perindopril, pindolol, phenoxybenzamine, phentolamine, polythiazide, prazosin, propranolol, quinapril, ramipril, spironolactone, telmisartan, terazosin, timolol, tolazoline, torsemide, trandolapril, triamterene, valsartan, and verapamil. In certain embodiments, the anti-hypertension agent is telmisartan.

Exemplary diagnostic agents include, but are not limited to, fluorescent molecules; gases; metals; imaging agents, such as commercially available imaging agents used in positron emissions tomography (PET), computer assisted tomography (CAT), single photon emission computerized tomography, x-ray, fluoroscopy, and magnetic resonance imaging (MRI); and contrast agents, such as magnetic-resonance signal enhancing agents. X-ray attenuating agents, ultrasound scattering agent, and ultrasound frequency shifting agents. Examples of suitable materials for use as contrast agents in MRI include gadolinium chelates, as well as iron, magnesium, manganese, copper, and chromium. Examples of materials useful for CAT and x-ray imaging include iodine-based materials. In certain embodiments, the diagnostic agent is used in magnetic resonance imaging (MRI), such as iron oxide particles or gadolinium complexes. Gadolinium complexes that have been approved for clinical use include gadolinium chelates with DTPA. DTPA-BMA, DOTA and HP-DO3A which are reviewed in Aime, et al., (Chemical Society Reviews (1998), 27:19-29), the entire teachings of which are incorporated herein by reference.

In certain embodiments, the diagnostic agent is a metal, inorganic compound, organometallic compound, organic compound, or salt thereof. In certain embodiments, the imaging agent contains a metal selected from the group consisting of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, gadolinium, gallium, thallium, and barium. In certain embodiments, the diagnostic agent is an organic compound. In certain embodiments, the diagnostic agent is metal-free. In certain embodiments, the diagnostic agent is a metal-free organic compound.

In certain embodiments, the imaging agent is a magnetic resonance imaging (MRI) agent. In certain embodiments, the MRI agent is gadolinium. In certain embodiments, the MRI agent is a nitroxide radical-containing compound.

In certain embodiments, the imaging agent is a nuclear medicine imaging agent. In certain embodiments, the nuclear medicine imaging agent is selected from the group consisting of ⁶⁴Cu diacetyl-bis(N⁴-methylthiosemicarbazone) (⁶⁴Cu-ASTM), ¹⁸F-fluorodeoxyglucose (FDG). ¹⁸F-fluoride, 3′-deoxy-3′-[¹⁸F]fluorothymidine (FLT), ¹⁸F-fluoromisonidazole (FMISO), gallium, technetium-99m, and thallium.

In certain embodiments, the imaging agent is radiographic imaging agent. In certain embodiments, the radiographic imaging agent is selected from the group consisting of barium, gastrografin, and iodine contrast agent.

In certain embodiments, the imaging agent is a radical-containing compound. In certain embodiments, the imaging agent is a nitroxide radical-containing compound. In certain embodiments, the imaging agent or diagnostic agent is of the formula:

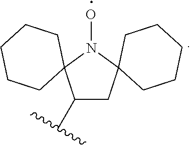

In certain embodiments, the imaging agent or diagnostic agent is an organic compound. In certain embodiments, the imaging agent is a salt of an organic compound. In certain embodiments, the imaging agent or diagnostic agent is of the formula:

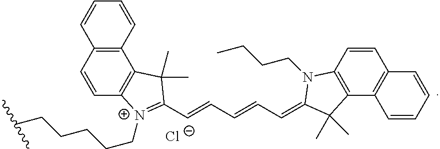 [SMILES: *CCCCC[N+]1=C(/C=C/C=C/C=C2\N(CCCC)c3ccc4ccccc4c3C2(C)C)C(C)(C)c2c1ccc1ccccc21 ; [Cl-]]

In certain embodiments, the diagnostic agent may comprise a fluorescent molecule, a metal chelate, a contrast agent, a radionuclide, or a positron emission tomography (PET) imaging agent, an infrared imaging agent, a near-IR imaging agent, a computer assisted tomography (CAT) imaging agent, a photon emission computerized tomography imaging agent, an X-ray imaging agent, or a magnetic resonance imaging (MRI) agent.

In some embodiments, the diagnostic agent is a fluorescent molecule. In some embodiments, the fluorescent molecule comprises an acridine dye, a cyanine dye, a rhodamine dye, a BODIPY dye, a fluorescein dye, a dansyl dye, an Alexa dye, an atto dye, a quantum dot, or a fluorescent protein. In some embodiments, the fluorescent molecule is a cyanine dye (e.g., Cy3, Cy 3.5, Cy5, Cy5.5, Cy7, or Cy7.5).

In some embodiments, the diagnostic agent is an MRI agent (e.g., a contrast agent). Examples of suitable materials for use as MRI agents (e.g., contrast agents) include gadolinium chelates, as well as iron, magnesium, manganese, copper, and chromium.

In some embodiments, the diagnostic agent is a CAT imaging agent or an X-ray imaging agent. Examples of materials useful for CAT and X-ray imaging include iodine-based materials.

In some embodiments, the diagnostic agent is a PET imaging agent. Examples of suitable PET imaging agents include compounds and compositions comprising the positron emitting radioisotopoes ¹⁸F, ¹⁵O, ¹³N, ¹¹C, ⁸²Rb, ⁶⁴Cu, and ⁶⁸Ga, e.g., fludeoxyglucose (¹⁸F-FDG), ⁶⁸Ga-DOTA-psuedopeptides (e.g., ⁶⁸Ga-DOTA-TOC), ¹¹C-metomidate, ¹¹C-acetate, ¹¹C-methionine, ¹¹C-choline, ¹⁸F-fluciclovine, ¹⁸F-fluorocholine, ¹⁸F-fluorodeoxysorbitol, ¹⁸F-3-fluoro-3′-deoxythymidine, ¹¹C-raclopride, and ¹⁸F-desmethoxyfallypride.

In some embodiments, the diagnostic agent is a near-IR imaging agent. Examples of near-IR imaging agents include Pz 247, DyLight 750, DyLight 800, cyanine dyes (e.g., Cy5, Cy5.5, Cy7), AlexaFluor 680, AlexaFluor 750, IRDye 680. IRDye 800CW, and Kodak X-SIGHT dyes.

In some embodiments, the agent can be a radionuclide, e.g., for use as a therapeutic, diagnostic, or prognostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming use with various embodiments of the present disclosure include, but are not limited to, ¹²³I, ¹²⁵I, ¹³⁰I, ¹³¹I, ¹³⁵I, ⁴⁷Sc, ⁷²As, ⁷²Sc, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, 6Ga, ⁶⁷Cu, ⁷⁵Br, ⁷⁷Br, ^{99m}Tc, ¹⁴C, ¹³N, ¹⁵O, ³²P, ³³P, or ¹⁸F.

Prophylactic agents that can be included in the conjugates of the disclosure include, but are not limited to, antibiotics, nutritional supplements, and vaccines. Vaccines may comprise isolated proteins or peptides, inactivated organisms and viruses, dead organisms and viruses, genetically altered organisms or viruses, and cell extracts. Prophylactic agents may be combined with interleukins, interferon, cytokines, and adjuvants such as cholera toxin, alum. Freund's adjuvant.

In certain embodiments, at least one instance of the agent comprises one or more boron-containing moieties before the agent is included in the compound, and at least one instance of the boron-containing moiety forms Ring A of Formula (I) or Ring B of Formula (II) when or after the agent is included in the compound. In certain embodiments, at least one instance of the boron-containing moieties is a boronic acid moiety (e.g., —B(OH)₂). In certain embodiments, at least one instance of the boron-containing moieties is a boronic ester moiety (e.g., —B(OH)—OR², —B(OR²)₂, wherein each instance of R² is independently substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, or an oxygen protecting group, or two instances of R² are joined to form substituted or unsubstituted heterocyclyl). In certain embodiments, the agent comprising one or more boron-containing moieties is bortezomib. In certain embodiments, the agent comprising one or more boron-containing moieties is tavaborole

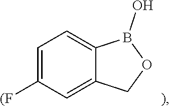

a boron-containing derivative of bocepevir

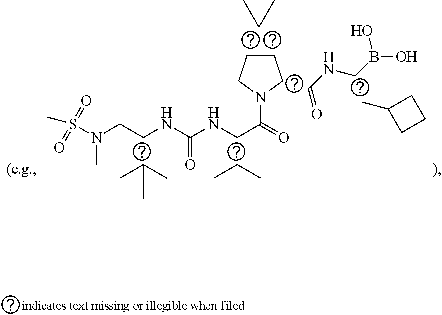

benzo[b]thiophene-2-boronic acid

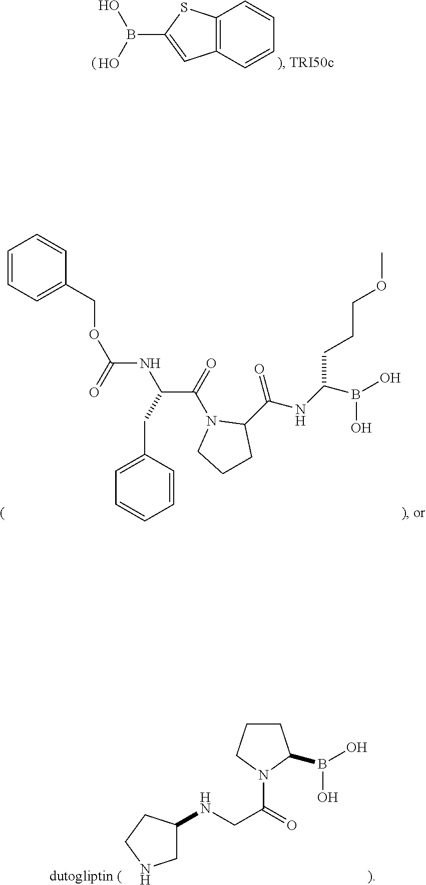 [SMILES: OB(O)c1cc2ccccc2s1 ; COCCC[C@@H](NC(=O)C1CCCN1C(=O)[C@H](Cc1ccccc1)NC(=O)OCc1ccccc1)B(O)O ; O=C(CN[C@@H]1CCNC1)N1CCC[C@H]1B(O)O]

In certain embodiments, when the agent comprising one or more boron-containing moieties, the agent corresponds to the formula:

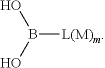

In certain embodiments, the boron atom included in Ring A of Formula (I) or in Ring C of Formula (II) is part of at least one instance of the agent, when the at least one instance of the agent comprises one or more boron-containing moieties before the agent is included in the compound.

In certain embodiments, the boron atom included in Ring A of Formula (I) or in Ring C of Formula (II) is not part of at least one instance of the agent. In certain embodiments, at least one instance of the agent does not comprise a boronic acid moiety or boronic ester moiety. In certain embodiments, at least one instance of the agent does not comprise a boron atom.

In certain embodiments, m is 1. In some embodiments, m is 2. In certain embodiments, m is 3.

In some embodiments, d is 1. In certain embodiments, d is 2. In certain embodiments d is 3.

In certain embodiments, at least two instances (e.g., two instances) of M are different from each other. In certain embodiments, at least three instances (e.g., three instances) of M are different from each other. In certain embodiments, at least four instances (e.g., four instances) of M are different from each other. In certain embodiments, at least one instance of M is an immunomoduratory agent (e.g., immunomodulatory imide drug), and at least one instance of M is an anti-cancer agent. In certain embodiments, at least one instance of M is thalidomide, lenalidomide, or pomalidomide, and at least one instance of M is bortezomib. In certain embodiments, at least one instance of M is thalidomide, at least one instance of M is pomalidomide, and at least one instance of M is bortezomib.

In certain embodiments, a compound of Formula I is of the formula:

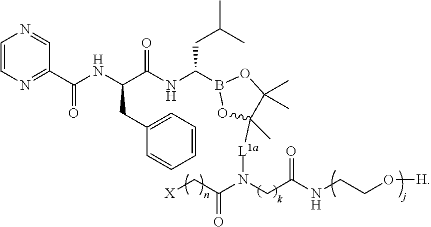

In some embodiments, a compound of Formula I is of the formula:

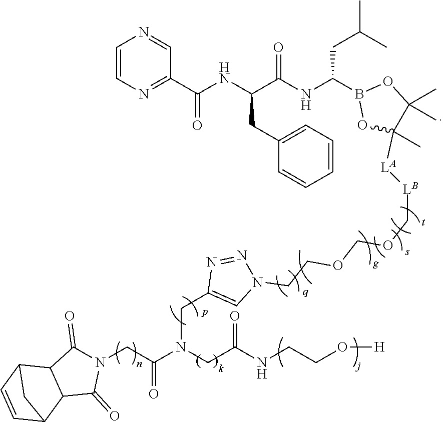

In certain embodiments, a compound of Formula I is of Formula I-1:

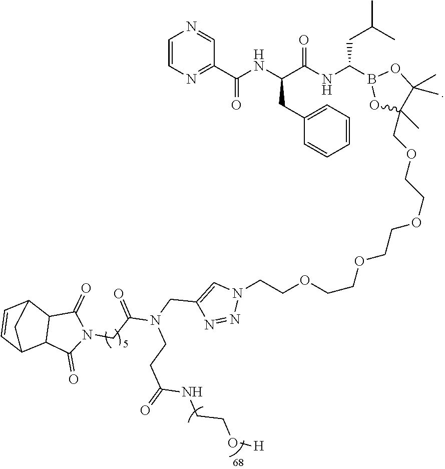

### Polymers

The present disclosure describes polymers (e.g., bottlebrush polymers (BBPs)) and materials produced from polymerizing monomers of Formula (I) and Formula (II). In certain embodiments, the polymer comprises one or more types of repeating units, wherein at least one type of the repeating units comprises a moiety of the formula:

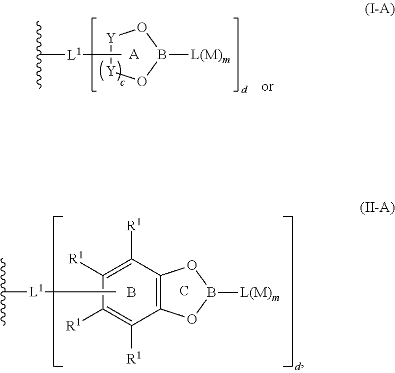

wherein:

L¹ is a substituted or unsubstituted linker, wherein the backbone of L¹ comprises two or more atoms;

each instance of Y is independently —C(R¹)₂—;

each instance of R¹ is independently absent, hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR, —CN, —SCN, —C(═NR^{a})R^{a}, —C(═NR)OR, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or two instances of R¹ are joined to form substituted or unsubstituted carbocyclyl or substituted or unsubstituted heterocyclyl;

each instance of R¹ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;

each instance of L is independently a bond or a substituted or unsubstituted linker, wherein the atom in the backbone of L attached to Ring A or Ring C is carbon;

each instance of M is independently an agent;

each instance of m is independently an integer between 1 and 10, inclusive;

each instance of c is independently an integer between 1 and 2, inclusive; and

d is an integer between 1 and 10, inclusive.

The moieties and variables in Formula (I-A) and (II-A) are as described herein.

The present disclosure also describes polymers prepared from monomers as described herein. In certain embodiments, a polymer contains a monomer as described herein. In some embodiments, a polymer comprises a first monomer and a second monomer as described herein, wherein all instances of M are the same. In some embodiments, a polymer comprises a first monomer and a second monomer as described herein, wherein at least one instance of M of the first monomer is different from at least one instance of M in the second monomer.

In certain embodiments, the polymer is a homopolymer. In certain embodiments, the polymer is a copolymer (e.g., a copolymer prepared by polymerizing two different types of monomers). In certain embodiments, the polymer is a linear polymer. In certain embodiments, the polymer is a linear copolymer (e.g., a block copolymer, alternating copolymer, periodic copolymer, statistical copolymer, stereoblock copolymer, gradient copolymer). In certain embodiments, the polymer is a branched polymer (e.g., branched copolymer). In certain embodiments, the polymer is a graft copolymer (e.g., star copolymer). In certain embodiments, the polymer is a regular copolymer. In certain embodiments, the polymer is a random copolymer. In certain embodiments, the polymer is a brush polymer. In certain embodiments, the polymer is a bottlebrush polymer. In certain embodiments, the polymer is a charged polymer. In certain embodiments, the polymer is a hydrophilic polymer. In certain embodiments, the polymer is a hydrophobic polymer.

In certain embodiments, the terms “polymer”, “conjugate”, and “particle” are used interchangeably. Exemplary conjugates or particles may be described by a number of properties, including. Mₙ=average molecular weight (kDa), D_{H}=average hydrodynamic diameter (nm), and PDI=polydispersity.

In certain embodiments, the Mₙ is determined with gel permeation chromatography, viscometry via the (Mark-Houwink equation), colligative methods (such as vapor pressure osmometry), end-group determination, or proton NMR. In certain embodiments, the M_{w} is determined with static light scattering, small angle neutron scattering. X-ray scattering, and sedimentation velocity. In some embodiments, the average molecular weight of the conjugate is between about 10 kDa and about 100 kDa, e.g., between about 15 kDa and about 85 kDa, about 20 kDa and about 60 kDa, or about 30 kDa and about 50 kDa, e.g., as determined by gel permeation chromatography. In one embodiment, the average molecular weight of the conjugate is between about 20 kDa and about 60 kDa. In one embodiment, the average molecular weight of the conjugate is between about 30 kDa and about 50 kDa.

In some embodiments, the average molecular weight of the conjugate is less than about 100 kDa (e.g., less than about 95 kDa, about 90 kDa, about 85 kDa, about 80 kDa, about 75 kDa, about 70 kDa, about 65 kDa, about 60 kDa, about 55 kDa, or about 50 kDa), e.g., as determined by gel permeation chromatography. In some embodiments, the average molecular weight of the conjugate is less than about 75 kDa (e.g., less than about 70 kDa, about 65 kDa, about 60 kDa, about 55 kDa, or about 50 kDa).

In some embodiments, the average molecular weight of the particle is between about 100 kDa and about 1,000 kDa, e.g., between about 200 kDa and about 700 kDa or about 300 kDa and about 500 kDa, e.g., as determined by gel permeation chromatography. In one embodiment, the average molecular weight of the particle is between about 2000 kDa and about 70 kDa. In one embodiment, the average molecular weight of the particle is between about 300 kDa and about 500 kDa.

In some embodiments, the average molecular weight of the particle is less than about 1,000 kDa (e.g., less than about 950 kDa, about 900 kDa, about 850 kDa, about 800 kDa, about 750 kDa, about 700 kDa, about 650 kDa, about 600 kDa, about 550 kDa, or about 500 kDa), e.g., as determined by gel permeation chromatography. In some embodiments, the average molecular weight of the particle is less than about 750 kDa (e.g., less than about 700 kDa, about 650 kDa, about 600 kDa, about 550 kDa, or about 500 kDa). In some embodiments, the average molecular weight of the particle is less than about 500 kDa (e.g., less than about 450 kDa, about 400 kDa, about 350 kDa, or 300 kDa).

In some embodiments, weight average molecular weight of the polymer is between 3,000 and 1,000,000, inclusive, g/mol. In certain embodiments, the weight average molecular weight of the polymer is between 3,000 and 300,000, inclusive, g/mol. In some embodiments, weight average molecular weight of the polymer is between 3,000 and 100,000, inclusive, g/mol. In some embodiments, weight average molecular weight of the polymer is between 3,000 and 10,000, inclusive, g/mol. In some embodiments, weight average molecular weight of the polymer is between 10,000 and 1,000,000, inclusive, g/mol. In some embodiments, weight average molecular weight of the polymer is between 10,000 and 100,000, inclusive, g/mol. In some embodiments, weight average molecular weight of the polymer is between 100,000 and 1,000,000, inclusive, g/mol. In some embodiments, weight average molecular weight of the polymer is between 10,000 and 50,000 inclusive, inclusive, g/mol. In some embodiments, weight average molecular weight of the polymer is between 20,000 and 50,000 inclusive, g/mol.

In some embodiments, the average hydrodynamic diameter of the conjugate is less than 50 nm (e.g., less than about 45 nm, about 40 nm, about 35 nm, about 25 nm, about 20 nm, about 15 nm, about 10 nm, about 7.5 nm, or less), e.g., as determined by dynamic light scattering. In some embodiments, the average hydrodynamic diameter of the conjugate is between about 1 nm and about 20 nm (e.g., between about 2.5 nm and about 17.5 nm, or about 5 nm and about 15 nm). In some embodiments, the average hydrodynamic diameter of the conjugate is between about 5 nm and about 15 nm.

In some embodiments, the average hydrodynamic diameter of the particle is less than 100 nm (e.g., less than about 90 nm, about 80 nm, about 75 nm, about 70 nm, about 65 nm, about 60 nm, about 55 nm, about 50 nm, about 45 nm, about 40 nm, about 35 nm, about 25 nm, or less), e.g., as determined by dynamic light scattering. In some embodiments, the average hydrodynamic diameter of the particle is between about 5 nm and about 100 nm (e.g., between about 7.5 nm and about 75 nm, about 10 nm and about 50 nm, about 12.5 nm and about 40 nm, or about 15 nm and about 30 nm). In some embodiments, the average hydrodynamic diameter of the particle is between about 10 nm and about 50 nm. In some embodiments, the average hydrodynamic diameter of the particle is between about 15 nm and about 30 nm.

In some embodiments, the average polydispersity of the conjugate or particle is less than about 0.5 (e.g., less than about 0.4, about 0.35, about 0.3, about 0.25, about 0.2, about 0.15, or less). In some embodiments, the average polydispersity of the conjugate or particle is less than about 0.3. In some embodiments, the average polydispersity of the conjugate or particle is less than about 0.2. In some embodiments, the conjugate or particle is monodisperse. In some embodiments, the conjugate or particle is about 50% monodisperse (e.g., about 55%, about 60%, about 65%, about 70%, about 75%, about 80%, about 85%, about 90%, about 95%, about 99%, or about 99.9% monodisperse).

In some embodiments, the conjugate or particle is substantially soluble in water (e.g., hydrophilic). In some embodiments, the conjugate or particle is substantially insoluble in water (e.g., hydrophobic). In some embodiments, the conjugate or particle is substantially insoluble in water and greater than about 10,000 parts water are required to dissolve 1 part polymer. In one embodiment, the conjugate or particle is amphiphilic. In one embodiment, the conjugate or particle comprises a segment that is hydrophobic and a segment that is hydrophilic.

The BBPs described herein may be able to deliver multiple agents ratiometrically and/or orthogonally. Different chemical and/or physical conditions may be employed to individually release the multiple agents upon delivery. The convergent synthesis of BBPs allow the attachment of different agents to the BBPs through different linkers (e.g., linkers cleavable by reduction, hydrolysis (such as esters), oxidation, and UV irradiation. The hydrolyzation, oxidation. UV irradiation, and reduction may be performed in any order and at the same time or different times.

In certain embodiments, the BBP is a polymer comprising at least 100 repeating units selected from the following formulae:

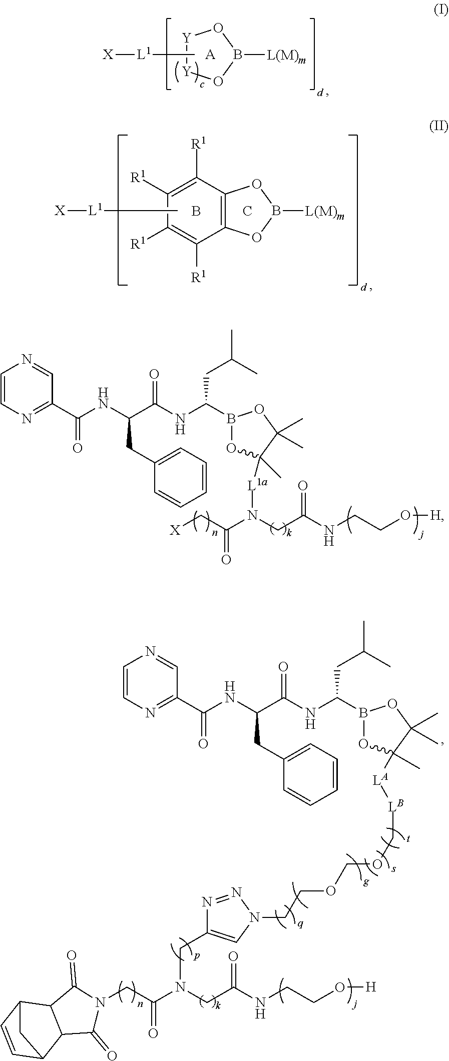

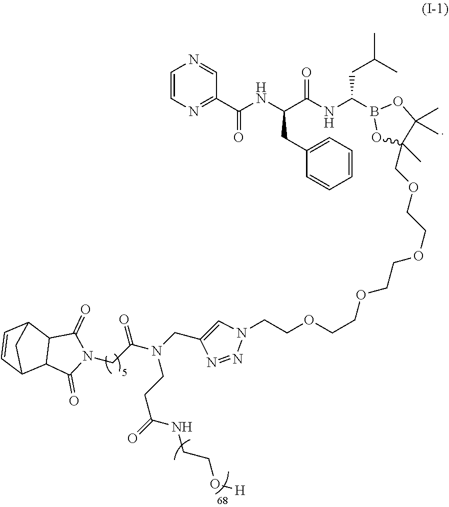

### Methods of Preparation of the Compounds and Polymers

The present disclosure describes methods of preparing polymers from monomers as described herein. In certain embodiments, the method of preparing a polymer comprises polymerizing a monomer as described herein.

The present disclosure also describes methods of preparing polymers comprising reacting an existing polymer with a compound described herein, wherein the existing polymer comprises a reaction handle able to react with X, and in the step of reacting, the reaction handle able to react with X is reacted with X. In certain embodiments, the existing polymer is a polymer described herein. In certain embodiments, the existing polymer is an addition polymer (e.g., polyethylene, poly(tetrafluoroethylene), polypropylene, polyisobutylene, polystyrene, polyacrylonitrile, poly(vinyl chloride), poly(methyl acrylate), poly(methyl methacrylate), polybutadiene, polychloroprene, poly(cis-1,4-isoprene), or poly(trans-1,4-isoprene)). In certain embodiments, the existing polymer is an condensation polymer (e.g., polyamide, polyaramide, polyester, polycarbonate, or silicone). In certain embodiments, the existing polymer is a poly(alkyl acrylate), a poly(hydroxyalkyl acrylate), poly(haloalkyl acrylate), polymethacrylate, a poly(alkyl methacrylate), a poly(hydroxyalkyl methacrylate), or a poly(haloalkyl methacrylate). In certain embodiments, the existing polymer is poly(methyl methacrylate). In certain embodiments, the existing polymer is polystyrene. In certain embodiments, the existing polymer is polyethylene. In certain embodiments, the existing polymer is a copolymer (e.g., a copolymer prepared by polymerizing two different types of monomers). In certain embodiments, the existing polymer is an alternating copolymer prepared by polymerizing two different types of substituted or unsubstituted ethenes.

In certain embodiments, the reaction handle able to react with X is a reaction handle described herein. In certain embodiments, the reaction handle able to react with X is an alkyne (e.g., —C≡CH). In certain embodiments, the reaction handle able to react with X is part (e.g., as a substituent on the backbone) of at least one type of the repeating units. In certain embodiments, at least 30%, at least 50%, at least 70%, at least 90%, at least 95%, or at least 99% of all instances of the reaction handle able to react with X is reacted with a compound described herein.

In some embodiments, the method of preparing a polymer comprises polymerizing a first monomer and a second monomer as described herein, wherein all instances of M are the same. In some embodiments, the method of preparing a polymer comprises polymerizing a first monomer and a second monomer as described herein, wherein at least one instance of M of the first monomer is different from at least one instance of M in the second monomer. In certain embodiments, an additional monomer is present in the step of polymerizing. In certain embodiments, the additional monomer is different from the monomer (e.g., the first monomer, the second monomer) described herein. In certain embodiments, substantially no additional monomer is present in the step of polymerizing.

In certain embodiments, X comprises a carboxylic acid, alcohol, and/or amine. In certain embodiments, a reagent for coupling a carboxylic acid with an alcohol or amine is N-(3-dimethylaminopropyl)-N′-ethylcarbodiimide (EDC), dicyclohexylcarbodiimide (DCC), 1-ethyl-3-(3-dimethylaminopropyl)carbodiimide hydrochloride (WSC/HCl), diphenylphosphorylazide (DPPA), carbonyldiimidazole (CDI), diethylcyanophosphonate (DEPC), benzotriazole-1-yloxy-trispyrrolidinophosphonium (DIPCI), benzotriazole-1-yloxy-trispyrrolidinophosphonium hexafluorophosphate (PyBOP), 1-hydroxybenzotriazole (HOBt), hydroxysuccinimide (HOSu), dimethylaminopyridine (DMAP), 1-hydroxy-7-azabenzotriazole (HOAt), hydroxyphthalimide (HOPht), pentafluorophenol (Pfp-OH), 2-(1H-benzotriazole-1-yl)-1,1,3,3-tetramethyluronium hexafluorophosphate (HBTU), O-(7-azabenzotriazole-1-yl)-1,1,3,3-tetramethyluronium hexafluorophosphonate (HATU), O-benzotriazole-1-yl-1,1,3,3-tetramethyluronium tetrafluoroborate (TBTU), or 3,4-dihydro-3-hydrodi-4-oxa-1,2,3-benzotriazine (Dhbt), or a salt thereof; or a combination (e.g., a combination of two) thereof. In certain embodiments, the reagent for coupling a carboxylic acid with an alcohol or amine is DCC. In certain embodiments, the reagent for coupling a carboxylic acid with an alcohol or amine is EDC, or a salt thereof.

The reagent for coupling a carboxylic acid with an alcohol or amine is used in an amount of about 1 to 20 equivalents of the compound of Formula (I) or Formula (II). In certain embodiments, the reagent for coupling a carboxylic acid with an alcohol or amine is used in an amount of about 1 to 10 equivalents. In certain embodiments, the activator is used in an amount of about 1 to 5 equivalents.

Any suitable solvent for coupling reactions can be used to perform coupling reactions described herein. Examples of useful solvents in the coupling reaction are DMSO, DMF, and methylene chloride. Additional exemplary solvents include acetonitrile, chloroform, tetrahydrofuran, and acetone.

The coupling reaction can be conducted at 0 to 50° C. In certain embodiments, the coupling reaction is conducted at room temperature for about 10 minutes to about 30 hours. In certain embodiments, the coupling reaction is conducted for about 15 minutes to about 24 hours.

In certain embodiments, the preparation of polymers as described herein comprises a conjugation reaction. For instance, EDC-NHS chemistry (1-ethyl-3-(3-dimethylaminopropyl) carbodiimide hydrochloride and N-hydroxysuccinimide), or a reaction involving a maleimide or a carboxylic acid, which can be conjugated to one end of a thiol, an amine, or a similarly functionalized polyether. The conjugation can be performed in an organic solvent, such as, but not limited to, methylene chloride, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting the agent that includes a hydroxyl, thiol, or amino group with a polymer comprising a carboxylic acid functional group. Such a reaction may occur as a single-step reaction, i.e., the conjugation is performed with or without using intermediates such as N-hydroxysuccinimide or a maleimide. The conjugation reaction between the amine-containing, thiol-containing, or hydroxyl-containing moiety and the carboxylic acid-terminated polymer may be achieved in one embodiment, by adding the amine-containing, thiol-containing, or hydroxyl-containing moiety, solubilized in an organic solvent such as, but not limited to, dichloromethane, acetonitrile, chloroform, tetrahydrofuran, acetone, formamide, dimethylformamide, pyridines, dioxane, or dimethylsulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously in some cases. Unconjugated monomers may be washed away after such reactions, and the polymer may be precipitated in solvents such as, for instance, ethyl ether, hexane, methanol, or ethanol.

In certain embodiments, the monomer contains a metathesis polymerization handle. In some embodiments, the polymer is prepared using a metathesis catalyst. In some embodiments, the metathesis catalyst is a transition metal metathesis catalyst. In certain embodiments, the methods for preparing the polymers (i.e., BBPs) described herein may involve a metathesis reaction. In certain embodiments, the metathesis reaction is a ring-opening metathesis polymerization (ROMP) (Liu et al., J. Am. Chem. Soc. 2012, 134,16337; Liu, J.; Gao, A. X.; Johnson, J. A. J Vis Exp 2013, e50874). In certain embodiments, the polymers described herein are prepared by polymerization of one or more monomers of Formula (I) and/or Formula (II) in the presence of a metathesis catalyst. The preparation methods described herein are versatile and have little limitations, e.g., in terms of the different agents that can be built into the BBPs. In certain embodiments, an agent that can be built into the BBPs includes functional groups that are compatible with ROMP.

In certain embodiments, the metathesis catalyst (e.g., ROMP catalyst) is a tungsten (W), molybdenum (Mo), or ruthenium (Ru) catalyst. In certain embodiments, the ROMP catalyst is a ruthenium catalyst. ROMP catalysts useful in the synthetic methods described herein include catalysts as depicted below, and as described in Grubbs et al., Acc. Chem. Res, 1995, 28, 446-452; U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299; the entire contents of each of which are incorporated herein by reference.

In certain embodiments, the ROMP catalyst is a Grubbs catalyst. In certain embodiments, the Grubbs catalyst is selected from the group consisting of:

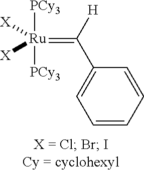

Benzylidenebis-(tricyclohexylphosphine)-dichlororuthenium (X=Cl); Benzylidenebis-(tricyclohexylphosphine)-dibromoruthenium (X=Br); Benzylidenebis-(tricyclohexylphosphine)-diiodoruthenium (X=I);

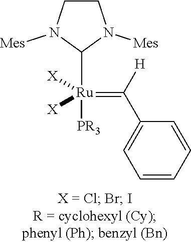

1,3-(Bis(mesityl)-2-imidazolidinylidene)dichloro-(phenylmethylene) (tricyclohexyl-phosphine)ruthenium (X=Cl; R=cyclohexyl); 1,3-(Bis(mesityl)-2-imidazolidinylidene)dibromo-(phenylmethylene) (tricyclohexyl-phosphine)ruthenium (X=Br; R=cyclohexyl); 1,3-(Bis(mesityl)-2-imidazolidinylidene)diiodo-(phenylmethylene) (tricyclohexyl-phosphine)ruthenium (X=I; R=cyclohexyl); 1,3-(Bis(mesityl)-2-imidazolidinylidene)dichloro-(phenylmethylene) (triphenylphosphine)ruthenium (X=Cl; R=phenyl); 1,3-(Bis(mesityl)-2-imidazolidinylidene)dichloro-(phenylmethylene) (tribenzylphosphine)ruthenium (X=Cl; R=benzyl);

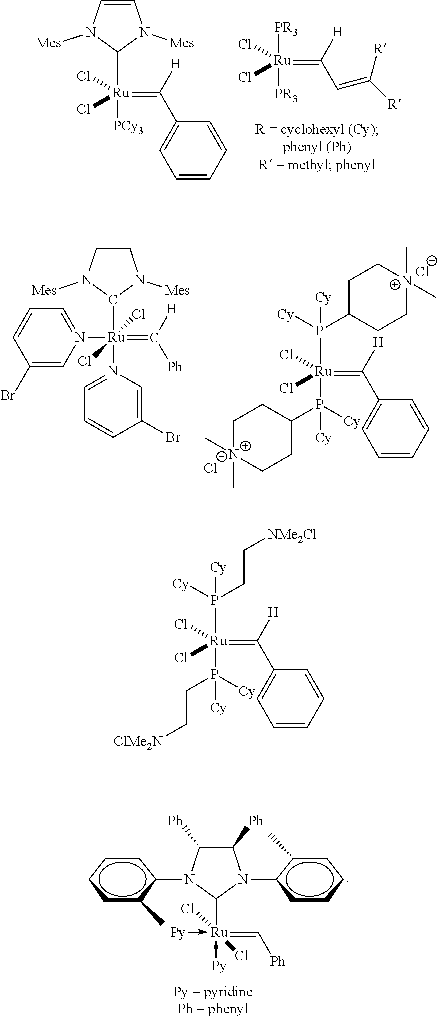

In certain embodiments, the ROMP catalyst is a Grubbs-Hoveyda catalyst. In certain embodiments, the Grubbs-Hoveyda catalyst is selected from the group consisting of:

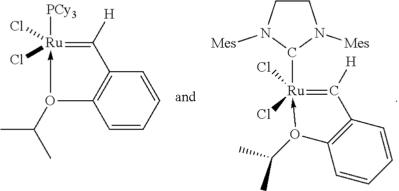

In certain embodiments, the ROMP catalyst is selected from the group consisting of:

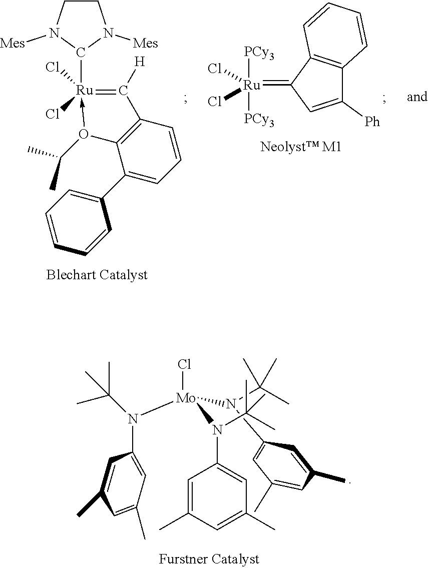

In certain embodiments, the ROMP catalyst is of the formula:

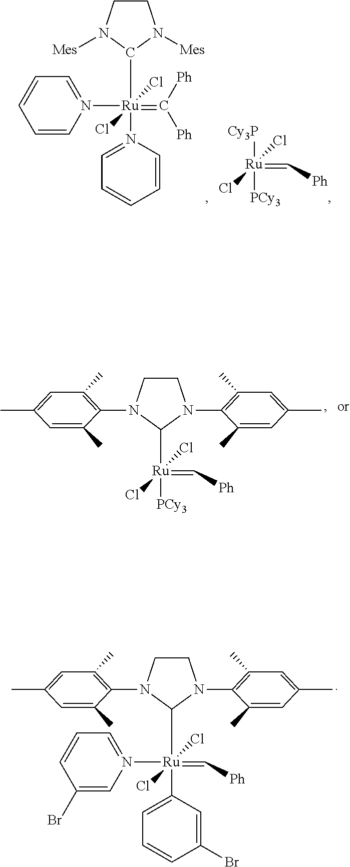

The ROMP can be conducted in one or more aprotic solvents. The term “aprotic solvent” means a non-nucleophilic solvent having a boiling point range above ambient temperature, preferably from about 25° C., to about 190° C., at atmospheric pressure. In certain embodiments, the aprotic solvent has a boiling point from about 80° C., to about 160° at atmospheric pressure. In certain embodiments, the aprotic solvent has a boiling point from about 80° C., to about 150° C., at atmospheric pressure. Examples of such solvents are methylene chloride, acetonitrile, toluene, DMF, diglyme. THE, and DMSO.

The ROMP can be quenched with a vinyl ether of the formula

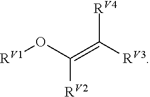

Each of R^{V1}, R^{V2}, R^{V3}, and R^{V4} is independently optionally substituted alkyl, optionally substituted alkenyl, optionally substituted alkynyl, optionally substituted carbocyclyl, optionally substituted phenyl, optionally substituted heterocyclyl, or optionally substituted heteroaryl. In certain embodiments, R^{V1} is optionally substituted alkyl, and R^{V2}, R^{V3}, and R^{V4} are hydrogen. In certain embodiments. R^{V1} is unsubstituted alkyl, and R^{V2}, R^{V3}, and R^{V4} are hydrogen. In certain embodiments, R^{V1} is substituted alkyl, and R^{V2}, R^{V3}, and R^{V4} are hydrogen. In certain embodiments, R^{V1} is methyl, and R^{V2}, R^{V3}, and R^{V4} are hydrogen. In certain embodiments, R^{V1} is ethyl, and R^{V2}, R^{V3}, and R^{V4} are hydrogen. In certain embodiments. R^{V1} is propyl, and R^{V2}, R^{V3}, and R^{V4} are hydrogen. In certain embodiments, R^{V1} is optionally substituted alkenyl, and R^{V2}, R^{V3}, and R^{V4} are hydrogen. In certain embodiments. R^{V1} is unsubstituted alkenyl, and R^{V2}, R^{V3}, and R^{V4} are hydrogen. In certain embodiments, R^{V1} is vinyl, and R^{V2}, R^{V3}, and R^{V4} are hydrogen. In certain embodiments, at least one of R^{V1}, R^{V2}, R^{V3}, and R^{V4} is conjugated with a diagnostic agent as defined above. In certain embodiments, the ROMP is quenched by ethyl vinyl ether. Excess ethyl vinyl ether can be removed from the BBPs by vacuum.

### Compositions and Kits

The present disclosure provides compositions (e.g., pharmaceutical compositions) comprising a polymer as described herein, and optionally an excipient (e.g., pharmaceutically acceptable excipient). In certain embodiments, the composition is a pharmaceutical composition. In certain embodiments, the excipient is a pharmaceutically acceptable excipient.

In certain embodiments, the pharmaceutical compositions are useful for delivering an agent (e.g., to a subject or cell). In certain embodiments, the pharmaceutical compositions are useful for treating a disease in a subject in need thereof. In certain embodiments, the pharmaceutical compositions are useful for preventing a disease in a subject. In certain embodiments, the pharmaceutical compositions are useful for diagnosing a disease in a subject.

In certain embodiments, the polymer described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a cancer in a subject in need thereof. In certain embodiments, the effective amount is an amount effective for preventing cancer in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of a protein kinase in a subject or cell. In certain embodiments, the effective amount is an amount effective for preventing a disease in a subject in need thereof. In certain embodiments, the effective amount is an amount effective for diagnosing a disease in a subject in need thereof.

In certain embodiments, the effective amount is an amount effective for delivering a pharmaceutical agent to a biological sample or cell. In certain embodiments, the cell is in vitro. In certain embodiments, the cell is in vivo. In certain embodiments, the cell is a malignant cell. In some embodiments, the cell is a premalignant cell.

Pharmaceutical compositions described herein can be prepared by any method known in the art of pharmacology. In general, such preparatory methods include bringing the polymer described herein (which may includes a therapeutic agent (the “active ingredient”)) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite. Glydant® Plus, Phenonip®, methylparaben. Germall® 115, Germaben® II. Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution. U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin. (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a polymer described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the polymer in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Polymers provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The polymers and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the polymer or pharmaceutical compositions described herein is suitable for topical administration to the eye of a subject.

The exact amount of a polymer required to achieve an effective amount will vary from subject to subject, depending, for example, on species, age, and general condition of a subject, severity of the side effects or disorder, identity of the particular polymer, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a polymer described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 pg and 1 pg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a polymer described herein. In certain embodiments, a dose described herein includes independently between 1 mg and 3 mg, inclusive, of a polymer described herein. In certain embodiments, a dose described herein includes independently between 3 mg and 10 mg, inclusive, of a polymer described herein. In certain embodiments, a dose described herein includes independently between 10 mg and 30 mg, inclusive, of a polymer described herein. In certain embodiments, a dose described herein includes independently between 30 mg and 100 mg, inclusive, of a polymer described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A polymer or composition as described herein, can be administered in combination with one or more additional pharmaceutical agents (e.g., therapeutically and/or prophylactically active agents). The polymer or composition can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in diagnosing a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a polymer described herein and an additional pharmaceutical agent shows a synergistic effect that is absent in a pharmaceutical composition including one of the polymer and the additional pharmaceutical agent, but not both.

The polymer or compositions can be administered concurrently with, prior to, or subsequent to one or more additional pharmaceutical agents, which are different from the polymer or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids. DNAs. RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the polymer or composition described herein in a single dose or administered separately in different doses. The particular combination to employ in a regimen will take into account compatibility of the polymer described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, antidiabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation.

In certain embodiments, the polymers described herein or pharmaceutical compositions can be administered in combination with an anti-cancer therapy including surgery, radiation therapy, transplantation (e.g., stem cell transplantation, bone marrow transplantation), immunotherapy, and chemotherapy. In certain embodiments, the polymers described herein or pharmaceutical compositions can be administered in combination with an additional therapy. In some embodiments, the polymers described herein or pharmaceutical compositions can be administered in combination with radiation therapy.

Also encompassed by the disclosure are kits (e.g., pharmaceutical packs). The kits provided may comprise a pharmaceutical composition or polymer described herein and instructions for use. The kits may further comprise a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or polymer described herein. In some embodiments, the pharmaceutical composition or polymer described herein provided in the first container and the second container are combined to form one unit dosage form.

In some embodiments, the percentage of the polymer that comprise an agent is between about 1 and about 100% (e.g., about 1%, about 2%, about 3%, about 4%, about 5%, about 10%, about 15%, about 20%, about 30%, about 40%, about 50%, about 60%, about 70%, about 80%, about 90%, or about 100%). In some embodiments, the percentage of the conjugates that comprise an agent is less than about 50%, e.g., less than about 40%, less than about 35%, less than about 30%, less than about 25%, less than about 20%, less than about 15%, or less than about 10%. In some embodiments, the percentage of the polymer that comprise an agent is between about 5% and about 50%, about 5% and about 40%, about 5% and about 30%, about 5% and about 25%, or about 5% and about 20%. In some embodiments, the percentage of the polymer that comprise an agent is between about 5% and 90%. In some embodiments, the percentage of the polymer that comprise an agent is between about 5% and about 75%. In the some embodiments, the polymer that comprise an agent is between about 5% and about 50%. In the some embodiments, the percentage of the polymer that comprise an agent is between about 10% and about 25%.

In some embodiments, the total amount of the agent present in the polymer is greater than about 5% (e.g., about 6%, about 7%, about 8%, about 9%, about 10%, about 12%, about 15%, about 20%, about 25%, about 30%, or more) of the total size or weight of the polymer. In some embodiments, the total amount of the agent present in the polymer is greater than about 10% (e.g., about 12%, about 15%, about 20%, about 25%, about 30%, or more) of the total size or weight of the polymer.

Without being bound by theory, the polymer disclosed herein may improve the efficiency of an agent by one or more of increasing the localization and/or release (e.g., preferential release) of the agent to a target cell (e.g., a cancer or a fibrotic cell; a cell associated with a hypoxic environment), or increasing the half life of the agent, thus resulting in a higher amount of a released agent at a target site (e.g., a tumor or liver (e.g., cirrhotic cell). According, the polymers disclosed herein can be more effective therapeutically than the free agent (e.g., due to enhanced drug uptake in the target tissue) and/or allow for a lower therapeutic dose of the agent, e.g., without substantially compromising the resulting drug concentration at a target tissue. In some embodiments, the polymers disclosed herein can reduce the adverse effect associated with systemic administration of an agent in free form (e.g., not coupled to polymer, conjugate or particle described herein).

Without being bound by theory, due to the localized delivery of the polymer (e.g., BBP) or compositions described herein, a lower dose or amount of the agent in the particles can be administered (e.g., through local sustained delivery) compared to the agent in free form. In other embodiments, the agent-containing particles are administered at a dose or amount of the agent that is less than the dose or amount of said agent in free form to have a desired effect (e.g., a desired therapeutic effect).

In some embodiments, the agent is incorporated into a polymer at a dose that is less than the dose or amount of said agent in free form to have a desired effect (e.g., a desired therapeutic effect), e.g., the standard of care dose for the intended use of the free agent. In one embodiment, the agent is incorporated into the particles at a dose or amount of the agent that is less than the standard of care dose of the agent for a desired therapy (e.g., a dose that is less than about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, or about 0.95 that of the standard of care dose of the agent).

In some embodiments, the agent is incorporated into a polymer at a dose equivalent to the dose or amount of said agent in free form to have a desired effect (e.g., a desired therapeutic effect), e.g., the standard of care dose for the intended use of the free agent. In these embodiments, the polymer produces a greater therapeutic effect and/or a less adverse effect than the free agent. In certain embodiments, the polymer increases the amount of the agent delivered to a tissue or cell in need thereof and reduces the amount of the agent exposed to a non-target tissue or cell, as compared to the free agent.

In some embodiments, the agent is incorporated into a polymer at a dose higher than the dose or amount of said agent in free form to have a desired effect (e.g., a desired therapeutic effect), e.g., the standard of care dose for the intended use of the free agent. In some embodiments, the agent is incorporated into a polymer at a dose higher than the dose or amount of said agent in free form that would produce an adverse effect by systemic administration (e.g., a reduction in blood pressure). In some embodiments, since the polymer described herein releases the agent at a target site based on pH microenvironment, other non-target sites (e.g., blood vessels) with different pH would be less likely to be exposed to the agent.

In another aspect, provided are kits including a first container comprising a polymer or pharmaceutical composition described herein. In certain embodiments, the kits are useful for delivering an agent (e.g., to a subject or cell). In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell. In certain embodiments, the kits are useful for diagnosing a disease in a subject or cell.

In certain embodiments, a kit described herein further includes instructions for using the kit. A kit described herein may also include information as required by a regulatory agency such as the U.S. Food and Drug Administration (FDA). In some embodiments, a kit comprises a polymer or composition as described herein and instructions for using the polymer or composition. In certain embodiments, the information included in the kits is prescribing information. In certain embodiments, the kits and instructions provide for delivering an agent. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods of Treatment and Uses

The present disclosure also provides methods of using the polymers described herein, or a pharmaceutical composition thereof, for delivering an agent. The present disclosure also provides methods of using the polymers described herein, or a pharmaceutical composition thereof, for the treatment, prevention, or diagnosis of a disease or condition.

The present disclosure provides methods of treating a disease in a subject in need thereof. In certain embodiments, the methods described herein comprise administering to a subject in need thereof a therapeutically effective amount of a polymer or composition. In some embodiments, the methods described herein comprise administering to a subject in need thereof a therapeutically effective amount of a polymer or composition, wherein at least one instance of M is a therapeutic agent. In certain embodiments, the disease is a proliferative disease. In some embodiments, the disease is cancer. In certain embodiments, the disease is lung cancer, head-and-neck cancer, esophagus cancer, stomach cancer, breast cancer, pancreas cancer, liver cancer, kidney cancer, prostate cancer, glioblastomas, metastatic melanomas, peritoneal or pleural mesotheliomas.

In certain embodiments, the methods described herein include administering to a subject with an effective amount of the polymers described herein, or a pharmaceutical composition thereof. In certain embodiments, the methods described herein include administering to a subject an effective amount of the polymers described herein, or a pharmaceutical composition thereof. In certain embodiments, the methods described herein comprise treating a disease or condition in a subject in need thereof by administering to in the subject a therapeutically effective amount of: a polymer described herein; or a pharmaceutical composition thereof; wherein at least one instance of M is a therapeutic agent. In certain embodiments, the methods described herein comprise preventing a disease or condition in a subject in need thereof by administering to the subject a prophylactically effective amount of: a polymer described herein; or a pharmaceutical composition thereof; wherein at least one instance of M is a prophylactic agent. In certain embodiments, the methods described herein comprise diagnosing a disease or condition in a subject in need thereof by administering to the subject a diagnostically effective amount of: a polymer described herein; or a pharmaceutical composition thereof; wherein at least one instance of M a diagnostic agent.

In certain embodiments, the disease or condition is a proliferative disease, hematological disease, neurological disease, painful condition, psychiatric disorder, metabolic disorder, or a long-term medical condition. In certain embodiments, the disease is cancer (e.g. lung cancer, large bowel cancer, pancreas cancer, biliary tract cancer, or endometrial cancer), benign neoplasm, angiogenesis, inflammatory disease, autoinflammatory disease, or autoimmune disease. In certain embodiments, the long-term medical condition is hypertension.

In some embodiments, the polymers described herein, or a pharmaceutical composition thereof are useful in treating a cancer. In some embodiments, the polymers described herein, or a pharmaceutical composition thereof, are useful to delay the onset of, slow the progression of, or ameliorate the symptoms of cancer. In some embodiments, the polymers described herein, or a pharmaceutical composition thereof, are administered in combination with other compounds, drugs, or therapeutics to treat cancer. In some embodiments, the polymers described herein, or a pharmaceutical composition thereof, are administered in combination with an additional therapy. In certain embodiments, the polymers described herein, or a pharmaceutical composition thereof, are administered in combination with an radiation therapy.

In some embodiments, the polymers described herein, or a pharmaceutical composition thereof are useful for treating a cancer including, but not limited to, acoustic neuroma, adenocarcinoma, adrenal gland cancer, anal cancer, angiosarcoma (e.g., lymphangiosarcoma, lymphangiocndothcliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL. T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL. T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides. Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM), a.k.a, myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the polymers described herein, or a pharmaceutical composition thereof, are useful in treating lung cancer, head-and-neck cancer, esophagus cancer, stomach cancer, breast cancer, pancreas cancer, liver cancer, kidney cancer, prostate cancer, glioblastomas, metastatic melanomas, peritoneal or pleural mesotheliomas. In some embodiments, the polymers described herein, or a pharmaceutical composition thereof, are used in combination with radiation therapy to treat lung cancer, head-and-neck cancer, esophagus cancer, stomach cancer, breast cancer, pancreas cancer, liver cancer, kidney cancer, prostate cancer, glioblastomas, metastatic melanomas, peritoneal or pleural mesotheliomas.

In some embodiments, the proliferative disease is a benign neoplasm. All types of benign neoplasms disclosed herein or known in the art are contemplated as being within the scope of the disclosure. In some embodiments, the proliferative disease is associated with angiogenesis. All types of angiogenesis disclosed herein or known in the art are contemplated as being within the scope of the disclosure. In certain embodiments, the proliferative disease is an inflammatory disease. All types of inflammatory diseases disclosed herein or known in the art are contemplated as being within the scope of the disclosure. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the disclosure. In some embodiments, the proliferative disease is an autoimmune disease. All types of autoimmune diseases disclosed herein or known in the art are contemplated as being within the scope of the disclosure.

In some embodiments, the polymers herein, or a pharmaceutical composition thereof contain at least one instance of M useful in treating cancer. In certain embodiments, M is a therapeutic agent. In certain embodiments, the therapeutic agent is an anti-cancer agent. In some embodiments, the anti-cancer agent is bortezomid. In some embodiments, the anti-cancer agent is selected from the group consisting of abiraterone acetate, ABVD, ABVE, ABVE-PC, AC, AC-T, ADE, ado-trastuzumab emtansine, afatinib dimaleate, aldesleukin, alemtuzumab, anastrozole, arsenic trioxide, asparaginase Erwinia chrysanthemi, axitinib, azacitidine. BEACOPP, belinostat, bendamustine hydrochloride. BEP, bevacizumab, bicalutamide, bleomycin, blinatumomab, bortezomib, bosutinib, brentuximab vedotin, busulfan, cabazitaxel, cabozantinib-s-malate, CAF, capecitabine, CAPOX, carboplatin, carboplatin-taxol, carfilzomibcarmustine, carmustine ceritinib, cetuximab, chlorambucil, chlorambucil-prednisone. CHOP, cisplatin, clofarabine, COPP, COPP-ABV, crizotinib, CVP, cyclophosphamide, cytarabine, dabrafenib, dacarbazine, dactinomycin, dasatinib, daunorubicin hydrochloride, decitabine, degarelix, denileukin diftitox, denosumab. Dinutuximab, docetaxel, doxorubicin hydrochloride, doxorubicin hydrochloride liposome, enzalutamide, epirubicin hydrochloride, EPOCH, erlotinib hydrochloride, etoposide, etoposide phosphate, everolimus, exemestane. FEC, fludarabine phosphate, fluorouracil, FOLFIRI, FOLFIRI-BEVACIZUMAB, FOLFIRI-CETUXIMAB, FOLFIRINOX, FOLFOX, FU-LV, fulvestrant, gefitinib, gemcitabine hydrochloride, gemcitabine-cisplatin, gemcitabine-oxaliplatin, goserelin acetate. Hyper-CVAD, ibritumomab tiuxetan, ibrutinib, ICE, idelalisib, ifosfamide, imatinib mesylate, imiquimod, ipilimumab, irinotecan hydrochloride, ixabepilone, lanreotide acetate, lapatinib ditosylate, lenalidomide, lenvatinib, letrozole, leucovorin calcium, leuprolide acetate, liposomal cytarabine, lomustine, mechlorethamine hydrochloride, megestrol acetate, mercaptopurine, methotrexate, mitomycin c, mitoxantrone hydrochloride, MOPP, nelarabine, nilotinib, nivolumab, obinutuzumab, OEPA, ofatumumab, OFF, olaparib, omacetaxine mepesuccinate. OPPA, oxaliplatin, paclitaxel, paclitaxel albumin-stabilized nanoparticle formulation, PAD, palbociclib, pamidronate disodium, panitumumab, panobinostat, pazopanib hydrochloride, pegaspargase, peginterferon alfa-2b, peginterferon alfa-2b, pembrolizumab, pemetrexed disodium, pertuzumab, plerixafor, pomalidomide, ponatinib hydrochloride, pralatrexate, prednisone, procarbazine hydrochloride, radium 223 dichloride, raloxifene hydrochloride, ramucirumab, R-CHOP, recombinant HPV bivalent vaccine, recombinant human papillomavirus, nonavalent vaccine, recombinant human papillomavirus, quadrivalent vaccine, recombinant interferon alfa-2b, regorafenib, rituximab, romidepsin, ruxolitinib phosphate, siltuximab, sipuleucel-t, sorafenib tosylate, STANFORD V, sunitinib malate, TAC, tamoxifen citrate, temozolomide, temsirolimus, thalidomide, thiotepa, topotecan hydrochloride, toremifene, tositumomab and iodine I 131, tositumomab, TPF, trametinib, trastuzumab, VAMP, vandetanib, VEIP, vemurafenib, vinblastine sulfate, vincristine sulfate, vincristine sulfate liposome, vinorelbine tartrate, vismodegib, vorinostat, XELIRI, XELOX, ziv-aflibercept, and zoledronic acid. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab). T-DM1, AVASTIN (bevacizumab), ERBITUX (cetuximab). VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel. Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g., 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g., mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca² ATPase inhibitors (e.g. thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®). ENMD-2076, PCI-32765, AC₂₂₀, dovitinib lactate (TK1258, CHIR-258). BIBW 2992 (TOVOK™), SGX523. PF-04217903. PF-02341066. PF-299804. BMS-777607. ABT-869, MP470. BIBF 1120 (VARGATEF®), AP24534. JNJ-26483327, MGCD265. DCC-2036, BMS-690154, CEP-11981, tivozanib (AV-951), OSI-930, MM-121, XL-184. XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca). BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the methods provided herein comprise administering a pharmaceutical agent to a subject in need thereof. In certain embodiments, the pharmaceutical agent is administered in order to deliver the pharmaceutical agent to a subject in need thereof. In some embodiments, the pharmaceutical agent is administered via a polymer or pharmaceutical composition thereof. In certain embodiments, a pharmaceutical agent is delivered to a biological sample. In some embodiments, a pharmaceutical agent is delivered to a cell. In certain embodiments, the biological sample or cell is in vitro. In some embodiments, the biological sample or cell is in vivo. In some embodiments, the cell is a malignant cell. In certain embodiments the cell is a premalignant cell. In some embodiments, the pharmaceutical agent is delivered to a biological sample or cell to treat a disease. In some embodiments, the pharmaceutical agent is delivered to a biological sample or cell to prevent a disease. In some embodiments, the pharmaceutical agent is delivered to a biological sample or cell to diagnose a disease.

### Examples

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Detailed Description of the Examples

### A Rational Design of Boronic Ester-Based Btz Prodrug.

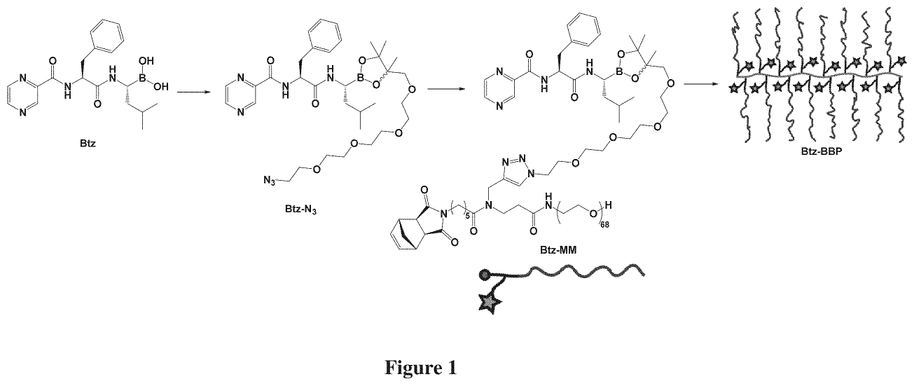

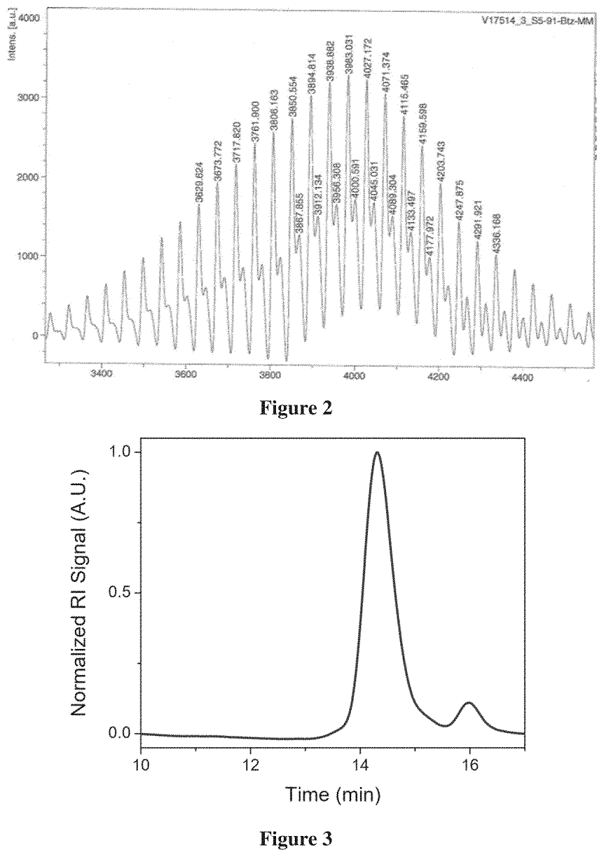

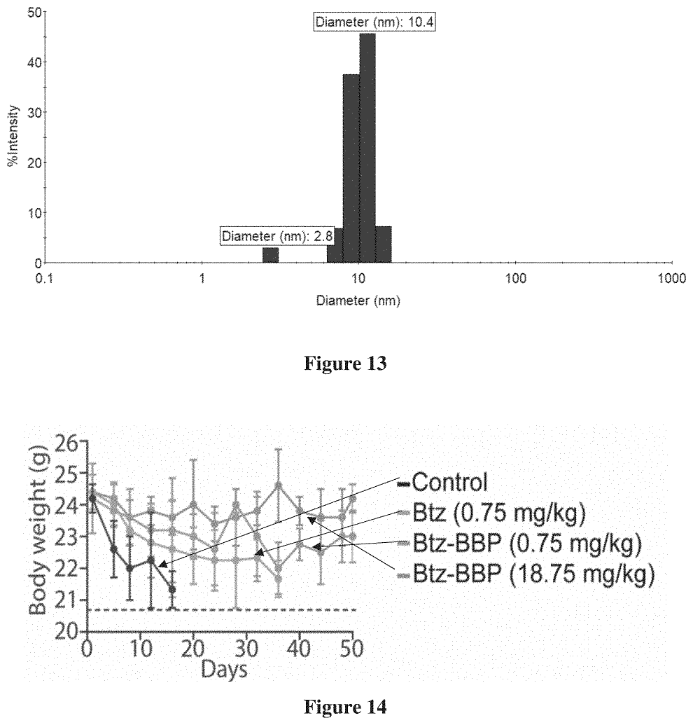

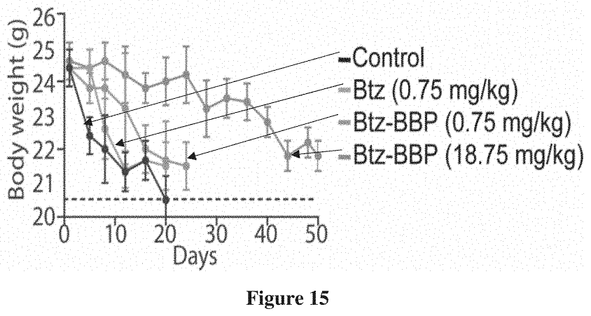

To construct a Btz-loaded BBP, linkers that would render Btz sufficiently stable and inactive during in vivo circulation, yet also enable “on-demand” release of Btz once inside the tumor microenvironment were first explored. The linker structure should be amendable to chemical modifications that would translate to tunable release rates depending on the end-application—an analogous design strategy to several of the reported drug conjugates.^{40,41} Hence, a 1,2-diol azide linker A5 was prepared (FIG. 1, full synthetic details described in the SI). A5 contains a tetraethylene glycol spacer for hydrophilicity, as well as a fully substituted diol for steric hindrance that would improve the Btz prodrug stability. Both factors were designed into the final linker via commercially available precursors (tetraethylene glycol and 2,3-dimethyl-2-butene, respectively), such that the linker structure can be conveniently modified to alter the release profile. Btz was then complexed onto A5 via boronic ester formation, resulting in the prodrug azide A6 (also known as Btz-N₃) (FIG. 1). Btz-loaded macromonomer was synthesized by copper-catalyzed alkyne-azide cycloaddition (CuAAC) “click” reaction of the azide onto the previously reported alkyne-functionalized macromonomer precursor.⁴⁵ The identities of all components were verified by ¹H and ¹³C nuclear magnetic resonance (NMR) spectroscopy and high-resolution mass spectrometry (HR-MS) and matrix-assisted laser desorption ionization time-of-flight (MALDI-ToF, FIG. 2) MS when appropriate. Btz-macromonomer was then subjected to ROMP, affording the final Btz-BBP (FIG. 1). These polymers were characterized by gel permeation chromatography (GPC, FIG. 3) and dynamic light scattering (DLS. FIG. 13), revealing efficient macromonomer-to-polymer conversion, as well as a targeted hydrodynamic diameter of 10±2 nm. For in vivo studies, a cyanine5,5-based macromonomer^{37-39,42} was also incorporated at 1% molar ratio to the Btz-macromonomer, affording a convenient way of tracking these BBPs in vivo by near-infrared fluorescence (NIRF) imaging.

### In Vitro Validation of Btz-BBP in MM Cell Lines.

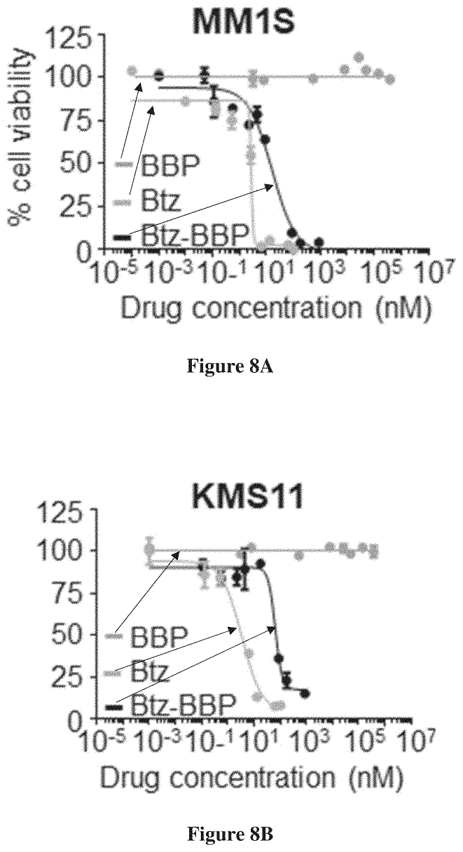

The potency of Btz-BBP was directly compared to free Btz in 2 MM cell lines (MM.1S, and KMS11). The effect on cell proliferation after 48 h of incubation with each agent was compared. Though Btz-BBP requires an additional step to release Btz from the BBP, both Btz and Btz-BBP displayed comparable effectiveness against MM.1S (IC₅₀=3.5±1.4 nM and 4.9±1.1 nM, respectively. FIG. 8A). This result implies the Btz-BBP has both high penetration and controlled release of Btz in the tumor cells. In an additional cell line, KMS11, Btz-BBP outperforms free Btz (IC₅₀=7.3+08 nM versus 29.9±1.6 nM for free Btz) (FIG. 8B). This increased efficacy could be due to the different mechanisms through which these two species are internalized into the cells: while free Btz utilizes membrane perfusion for penetration, the polymer uptake is dictated by endocytosis. With the latter, an efflux pump mechanism can be avoided, leading to higher intracellular levels of Btz.

### In Vivo Toxicity of Btz-BBP in Healthy BALB/c Mice.

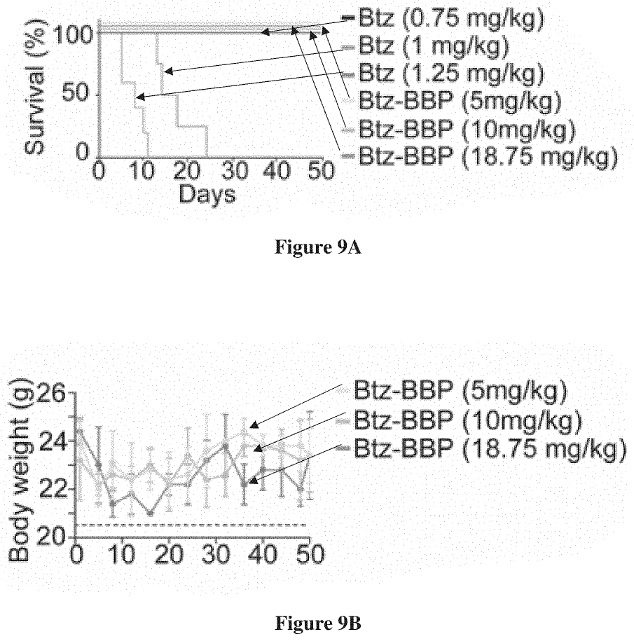

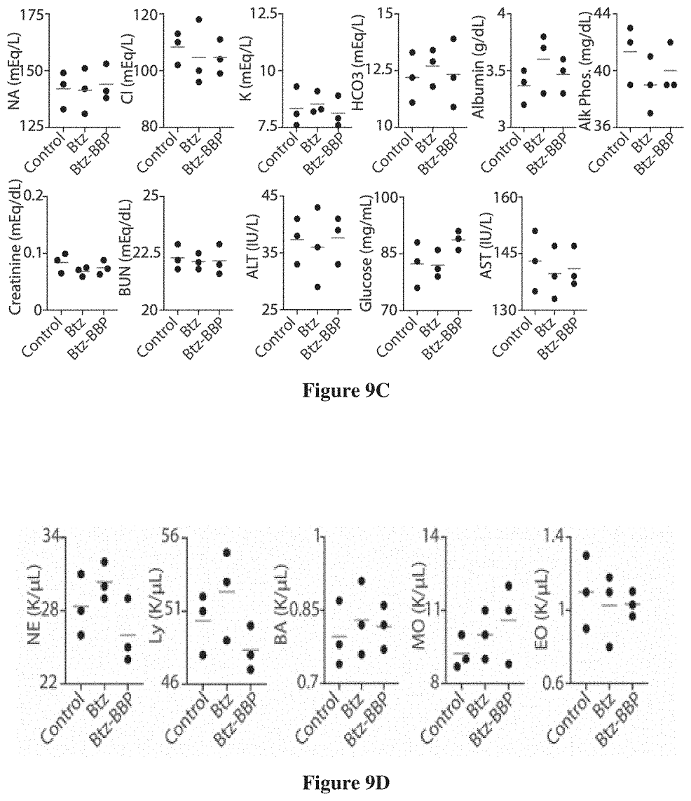

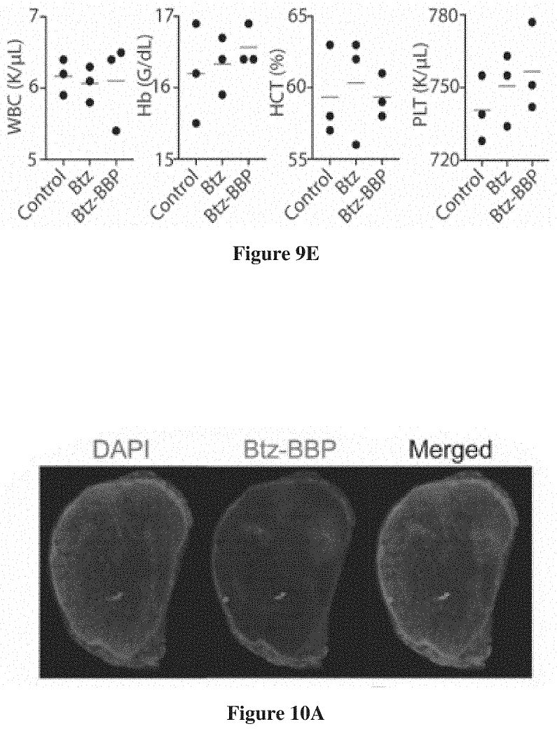

Having verified the retention of Btz activity of Btz-BBP in vitro, it was next sought to examine the capability of the BBP platform to minimize Btz toxicity in vivo by virtue of its design as discussed above. To this end, the in vivo toxicity profile of Btz (0.75, 1, 1.25 mg/kg, twice a week) and the Btz-BBP (5, 10, and 18.75 mg/kg, 2 times a week) in healthy BALB/c mice was performed (FIG. 9A). The Btz-free polymer was not examined in this study, as it has been previously established to be well-tolerated up to 2 g/kg.³⁷ For free Btz, doses of 0.75 mg/kg was previously established to be safe with for the animals, consistent with the results (FIG. 9A).¹⁶ Higher doses, though, confirm the toxicity of the drug, as reflected in a rapid decrease of the survival rates. In contrast, Btz-BBP remained well-tolerated at up to 18.75 mg/kg, a 25-fold improvement over its parent drug. Neither death, significant body weight loss (FIG. 9B), nor any signs of toxicity were observed. These results strongly suggest that the Btz remain conjugated to the BBP in the absence of the tumor microenvironment, enabling a much larger dose to be administered.

In parallel, a toxicology study was performed in BALB/c mice based on the same dosing schedule (2 injections per week over 4 weeks) to evaluate the changes induced by Btz-BBP on the metabolic profile (FIG. 9C), the complete blood counts (FIG. 9D), and the white blood cell differential counts (FIG. 9E). Mice were treated with either Btz (0.75 mg/kg) or Btz-BBP (18.75 mg/kg) at their respective MTD, and the tests were performed 2 weeks post-treatment. Compared to the PBS controls, mice exposed to Btz-BBP did not present any signs of toxicity (two-tailed student t-test, P>0.05). Altogether, it was concluded that Btz-BBP compound is well-tolerated, with a 25-fold enhancement in MTD compared to its parent Btz.

### Btz-BBP Improves Therapeutic Outcomes in MM Mouse Models

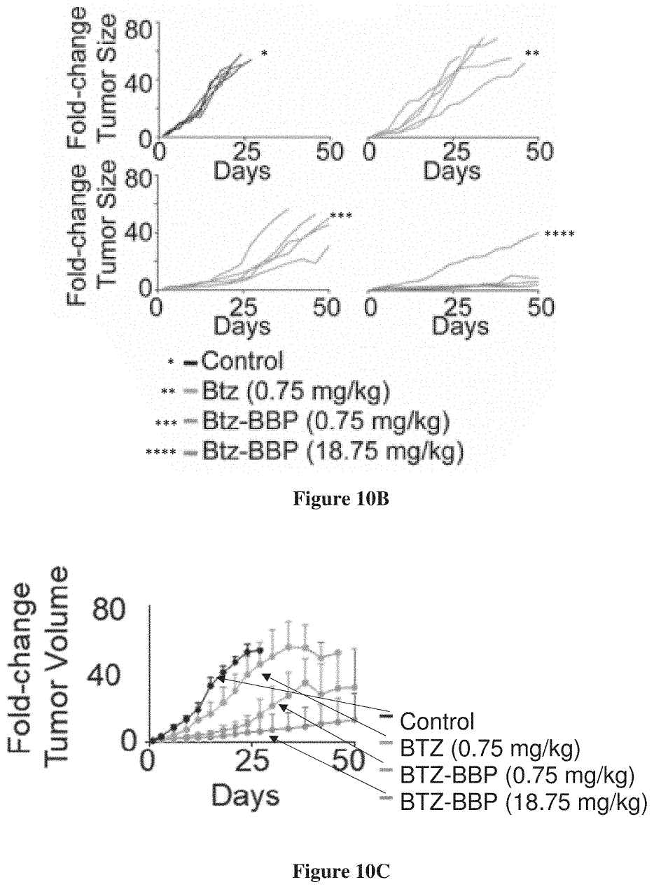

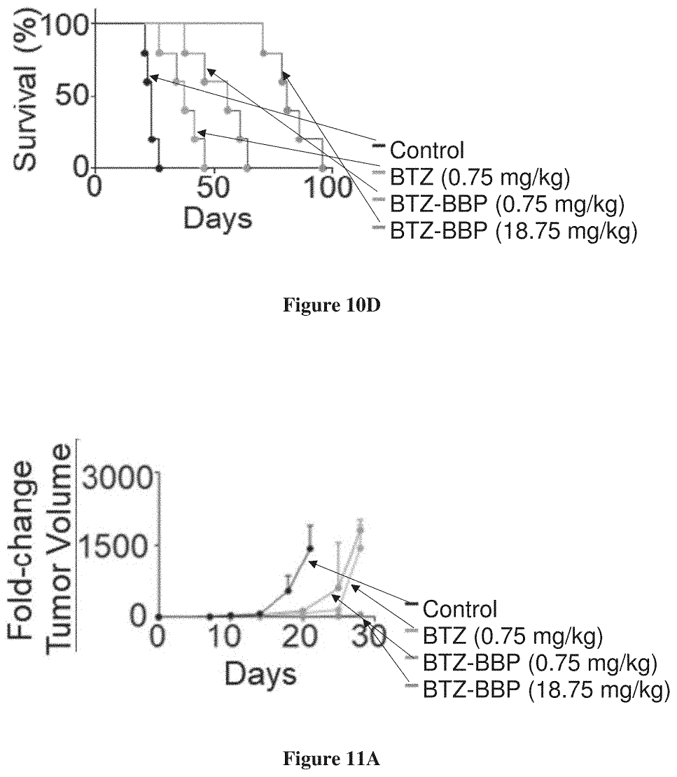

It was hypothesized that when released at the tumor site. Btz-BBP can provide a large enhancement in term of efficacy, constituting an advancement in Btz's TI that would merit further translations. An efficacy comparison between Btz and Btz-BBP was performed in two distinct animal models of MM. First, in a subcutaneous model of KMS11 (FIGS. 10A to 10D), Btz-BBP was administered to evaluate its ability to accumulate at the tumor tissue via passive uptake. At 1 hour post-injection, the tumors were harvested and examined under fluorescence microscopy, revealing Btz-BBP accumulation as well as deep penetrations into the tumor tissue (FIG. 10A).

Encouraged by this result, an efficacy study using this subcutaneous model was set up, which was composed of mice (n=5) treated with either PBS. Btz at MTD (0.0.75 mg/kg twice a week, intraperitoneal injection), Btz-BBP at dose-match (0.75 mg/kg, intravenous injection), and at Btz-BBP's MTD (18.75 mg/kg, intravenous injection). Treatments were administered over a period of 50 days, after which tumor were left to freely progress. Compared to the control, both Btz, dose-match Btz-BBP, and high dose of Btz-BBP demonstrated an improved therapeutic outcome. However, free Btz, even at its MTD, provided little efficacy with slight retardations of tumor progression, and the improved mean survival time that follows (42±6 days compared to the control group of 22±5 days) (FIGS. 10B to 10D). Interestingly, even at the same dose, Btz-BBP afforded therapeutic outcomes that outperformed its parent drug, reflected in both tumor progressions and enhanced survival rate (mean survival time of 61±9 days) (FIGS. 10B to 10D). This is due to the improved pharmacokinetics and tumor accumulation brought forth by the DDS, allowing a higher Btz concentration at the tumor site over longer exposure times. Furthermore, at 18.75 mg/kg, Btz-BBP afforded an almost complete response in all mice, as well as a survival benefit (mean survival time of 84±13 days, P<0.01).

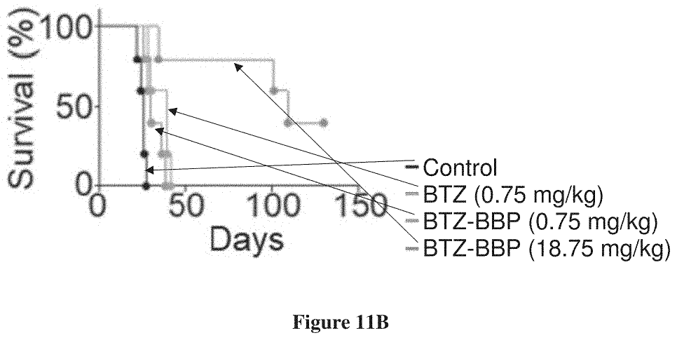

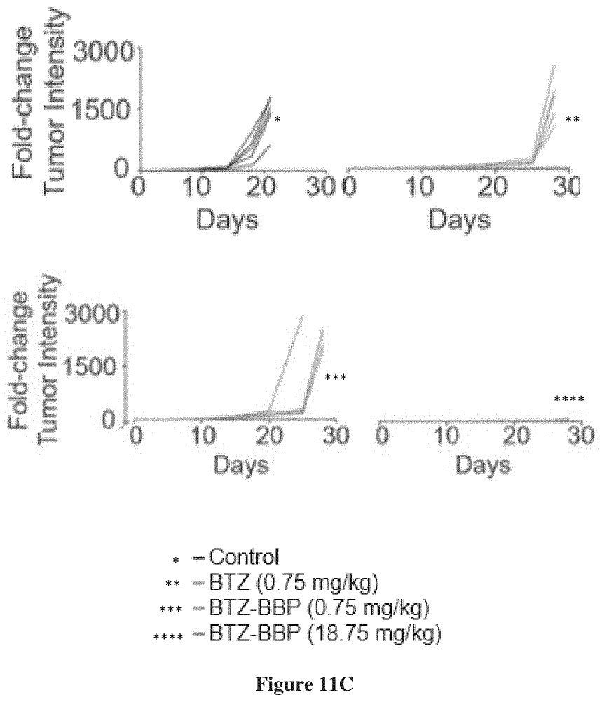

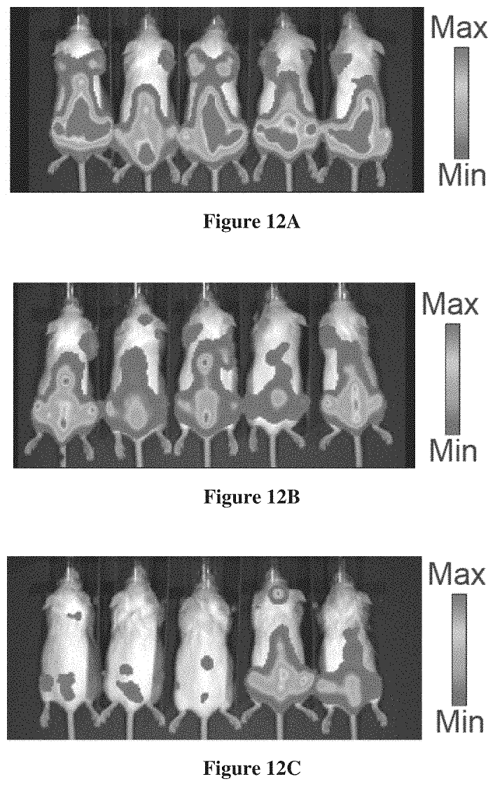

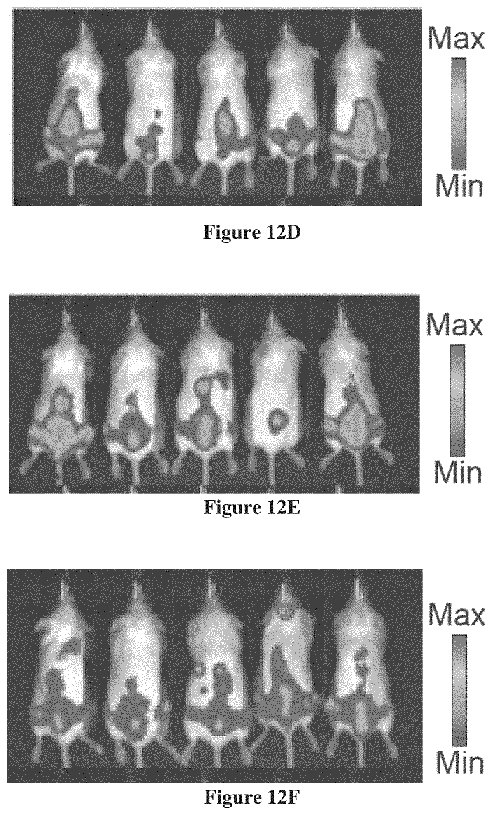

Motivated by these promising results, the next aim was to validate the DDS in an orthotopic model of MM, which primarily develops in the bone marrow of the animal. The tumors were induced via intravenous injection of MM.1S_{Luc+/GFP+} cells, and tumor progression was quantified by bioluminescence via the luciferase signal expressed by the MM.1S cells (FIGS. 12A to 12F). The study endpoint was reached once the animal exhibited hind limb paralysis or loss of >20% of their initial body weight. Mice (n=5) were treated for 30 days with the same treatment groups and dosing schedule described in the previous tumor model. Further, diminished improvements were observed for both free Btz MTD and Btz-BBP at dose-match (FIGS. 11A to 11C). While these groups treated with free drug still showed minor therapeutic efficacy values (FIGS. 11A to 11C), one can observe here the case where the TI had become exceedingly narrow that side effects became too problematic prior to reaching an effective dose which would result in an appreciable treatment outcome—a problem that is unfortunately not uncommon in the field for many free drugs. In stark contrast, at Btz-BBP's 25-fold higher MTD, tumor suppression and survival benefits (mean survival time of 108±11 days compared to the control group of 24±4 days) was demonstrate with complete response observed in 2 out of 5 mice (40%) (FIGS. 11A to 11C). In both models, the utilization of the BBP platform resulted in therapeutic outcomes that outperformed the standard Btz, the current first-line treatment in the clinic.

### Conclusion

In conclusion, the synthesis, characterization, in vitro and in vivo evaluation of a Btz-conjugated DDS has been reported. By leveraging the boronic acid of Btz, a boronic ester-based macromolecular prodrug was synthesized, namely Btz-BBP. Leveraging its unique designs and nanoarchitecture, Btz-BBP afforded improved stability, pharmacokinetics and tumor accumulation, as well as “on-demand” release in the tumor microenvironment. These advantages in turn afforded a much higher tolerability, and a 20-fold enhancement in MTD. Consequently, this lowered toxicity enabled a dose escalation that demonstrated therapeutic benefits over its parent drug, constituting a marked improvement in TI. Altogether, these results demonstrated the potential of Btz, a powerful PI plagued with adverse side effects; its incorporation onto the BBP platform therefore afforded a glimpse into how this class of therapeutics could be further leveraged as well as the benefits of a markedly improved TI.

### Materials/General Methods/Instrumentation

All reagents were purchased from commercial suppliers and used without further purification unless otherwise stated. Grubbs 3^{rd} generation bispyridyl catalyst G3-cat,⁴⁶ linker precursor A2,³⁶ macromonomer precursor yne-MM,⁴⁵ Cy-MM,³⁷ and A1⁴⁷ were prepared as previously reported.

Liquid chromatography mass spectrometry (LC/MS) was performed on an Agilent 1260 LC system equipped with a Zorbax SB-C₁₈ rapid resolution HT column using a binary solvent system (MeCN and H₂O with 0.1% CH₃COOH). Recycling preparative HPLC was performed on a LaboACE system (Japan Analytical Industry) using a JAIGEL-2 HR JAIGEL-2.5HR column in series. Gel permeation chromatography (GPC) analyses were performed on an Agilent 1260 Infinity setup with two Agilent PL1110-6500 columns in tandem and a 0.025 M LiBr DMF mobile phase run at 60° C. The differential refractive index (dRI) of each compound was monitored using a Wyatt Optilab T-rEX detector; and, the light scattering (LS) signal was acquired with a Wyatt Dawn Heleos-I detector. Column chromatography was carried out on silica gel 60F (EMD Millipore, 0.040-0.063 mm) or on aluminum oxide (Sigma-Aldrich, activated, neutral, Brockmann Activity I).

Nuclear magnetic resonance (NMR) spectra were recorded on Bruker AVANCE III-400 spectrometer, with working frequencies of 400 (¹H), and 100 (¹³C) MHz, or AVANCE-600 spectrometer with working frequencies of 600 (¹H), and 151 (¹³C) MHz. Chemical shifts are reported in ppm relative to the signals corresponding to the residual non-deuterated solvents: CDCl₃: δH=7.26 ppm and δC=77.16 ppm. High-resolution mass spectra (HRMS) were measured on a JEOL AccuTOF LC-Plus 4G with an IonSense DART. Matrix-assisted laser desorption/ionization time-of-flight (MALDI-TOF) analyses were collected on a Bruker OmniFlex instrument using sinapinic acid as the matrix.

Dynamic light scattering (DLS) measurements were performed on a Wyatt Technology Mobius DLS instrument. Nanoparticle suspensions were prepared in a solution of nanopure water (MilliQ). PBS buffer, or 5% v/v glucose/nanopore water (1 mg/mL). The resulting suspensions were passed through a 0.45 μm Nalgene filter (PES membrane) into disposable polystyrene cuvettes, which were pre-cleaned with compressed air. Measurements were made in sets of 10 acquisitions; and, the average hydrodynamic diameters were calculated using the DLS correlation function via a regularization fitting method (Dynamics 7.4.0.72 software package from Wyatt Technology).

Cell Lines.

KMS11 and MM.1S cells were provided by ATCC (Manassas, Va., USA).

Both cell lines were cultured in RPMI media (ThermoFisher Scientific) supplemented with 10% FBS (VWR), 1% Penicillin/Streptomycin (ThermoFisher Scientific), and 1% glutamine (ThermoFisher Scientific). MM.1S_{Luc}_{⁺}_{/GFP}_{⁺} cells were generated by retroviral transduction and authenticated by short tandem repeat DNA profiling. All cell lines were confirmed to be mycoplasma negative using the kit MycoAlert Mycoplasma (Lonza). Cell lines were housed in 5% CO₂ and 37° C., incubators.

In Vitro Assay.

Cells were plated at 10,000 cells/well in a 96 well plate overnight and then treated for 48 hours with either bortezomib (Btz) or Btz-bottlebrush (BBP) at various concentrations. Cell viability assay was performed using the Celltiter glo reagent kit (Promega).

Animal Usage.

All experiments involving animals were reviewed and approved by the Dana-Farber Cancer Institute Committee for Animal Care.

In Vivo Toxicity Evaluation.

Healthy BALB/c mice (n=5/group) were treated with either PBS, free Btz (0.75, 1, 1.25 mg/kg), or Btz-BBP (5, 10, 18.75 mg/kg) twice a week, 4 weeks in total. Drugs were injected subcutaneously for Btz and intravenously for Btz-BBP. Mice were euthanized if their body weight reached a 20% loss.

Metabolic profile, complete blood counts, and white blood cell differential counts were performed on mice (n=3/group) treated over 2-week period (2 injections per week) and left untreated for 2 or more weeks. Blood was collected by retro-orbital bleeding.

Animal models and survival study. The subcutaneous model of MM was generated with an injection of 3 million KMS11 cells in the hind flank of NCR nude mice. Cells were cultured as previously described. Tumor growth was monitor by caliper measurements (tumor measurement. TM). Once the tumor reached 1 cm in diameter, mice were randomly attributed to a treatment group. Btz free drug injection was administered subcutaneously, and Btz-BBP was injected intravenously. Drug was injected twice a week for a period of 4 weeks (8 injections in total) after mice were enrolled in the study. Mice body weight were tracked till day 50 (body weight measurement, BWM). Animals were sacrificed once the tumor reached 2 cm in the longest axis.

The orthotopic model of MM was obtained by injecting intravenously 1.5 million MM.1S_{Luc}_{⁺}_{/GFP}_{⁺} cells in the tail vain of SCID/beige mice. Tumor dissemination was assessed by IVIS imaging (IVIS Spectrum. Perkin Elmer, bioluminescence imaging, BLI) once a week. As soon as a signal was observed in the spine of the animal, the mice were randomly attributed to the treatment group stated above. Drug was injected twice a week for a period of 2 weeks (4 injections in total). Tumor burden response was obtained by IVIS imaging performed once a week. After 25 days, treatment was stopped. Endpoint criteria of the study was hind limb paralysis or loss of >20% of their initial body weight.

Statistical Analysis.

All statistical analysis was performed with GraphPad Prism Software (V.8.1.0)

### Synthetic Procedures: ROMP

Synthesis of A1

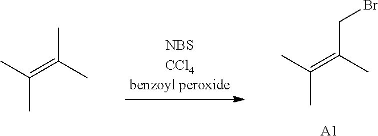

To an ice bath cooled solution of 2,3-dimethyl-2-butene (5.0 g, 59.4 mmol) in CCl₄ (200 mL) was added N-Bromosuccinimide (10.6 g, 59.4 mmol) and benzoyl peroxide (432 mg, 1.8 mmol). The reaction was allowed to warm up to room temperature and left to react overnight. After overnight reaction, the white powder was filtered out of solution and the collected solution was carefully rotovaped to remove CCl₄. The remaining crude mixture was then distilled to purify the desired CCl₄ was carefully removed by rotovap, and the bromoalkene was purified by distillation and obtained with 36% yield (3.45 g, 21.2 mmol, 36%). ¹H NMR (500 MHz, CDCl₃) δ (ppm) 4.08, 1.81-1.74, 1.71.

Synthesis of A3

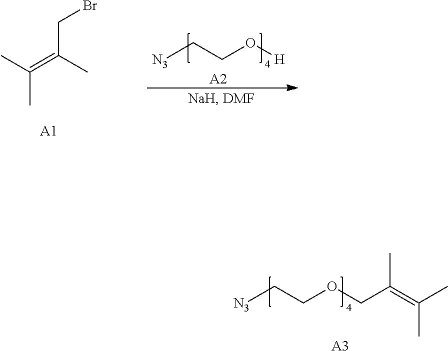

A1 (1.5 g, 9.1 mmol) was added to a solution of A2 (1.13 g, 4.55 mmol) in 10 mL of anhydrous DMF under N₂. The solution was cooled in an ice bath and then NaH (227 mg, 5.46 mmol) was added to the reaction mixture portionwise. Reaction was left stirring overnight, 5 mL of MeOH was added to quench the reaction. EtOAc was added and the solution was extracted 3 times with 5% LiCl solution. Column chromatography resulted in 80% yield of the product A3 (1.12 g, 3.7 mmol). ¹H NMR (500 MHz, CDCl₃) δ (ppm) 4.02, 3.69-3.64, 3.53, 3.40, 1.73, 1.70, 1.69.

Alternative Synthesis of A3

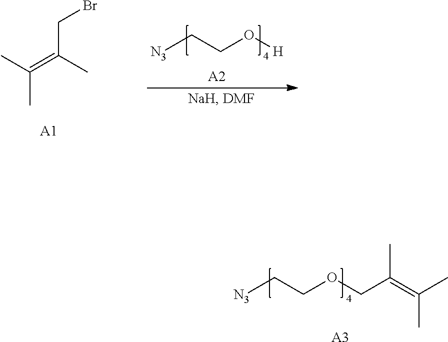

A1 (1.5 g, 9.1 mmol), obtained through a literature procedure,⁵ was added to a solution of A2 (1.13 g, 4.55 mmol) in anhydrous dimethylformamide (DMF, 20 mL) under N₂. The solution was cooled in an ice bath and then sodium hydride (NaH, 227 mg, 5.46 mmol) was added to the reaction mixture portionwise. The reaction mixture was left stirring overnight, 5 mL of MeOH was then added to quench the reaction. Ethyl acetate (EtOAc, 200 mL) was added and the solution was extracted 1 time with 200 mL of water. The organic layer was collected, dried over Na₂SO₄, and concentrated under vacuum. The crude mixture was then subjected to column chromatography (50/50 DCM/Hexanes to 1% MeOH in DCM), affording A3 (1.12 g, 3.7 mmol, 80% yield). HRMS-DART: Calcd for C₁₄H₂₈N₃O₄: m/z=302.2074 [M+H]⁺; Found: 302.2102. ¹H NMR (500 MHz, CDCl₃) δ (ppm) 4.01 (s, 2H), 3.69-3.63 (overlap, 12H), 3.53 (t, J=4.5 Hz, 2H), 3.39 (t, J=4.5 Hz, 2H), 1.72 (s, 3H), 1.70 (s, 3H), 1.69 (s, 3H). ¹³C NMR (125 MHz, CDCl₃) δ (ppm) 130.2, 125.0, 71.8, 70.8, 70.8, 70.7, 70.7, 70.1, 68.8, 50.8, 20.9, 20.2, 16.7.

Synthesis of the A4

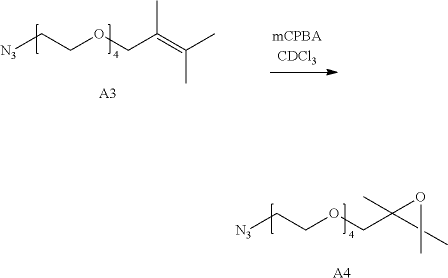

A3 (1.1 g, 3.58 mmol) was dissolved in 5 mL of CHCl₃ and the solution was cooled in an ice bath, mCPBA (1.20 g, 5.37 mmol) was added and the reaction was slowly warmed to 45° C. After overnight reaction, the solution was filtered to remove the white precipitate. The solution was concentrated and the crude A4 mixture used without further purification for the next step. ¹H NMR (500 MHz, CDCl₃) δ (ppm) 3.69-3.66, 3.61, 3.53, 3.40, 1.39, 1.35, 1.34.

Synthesis of A5

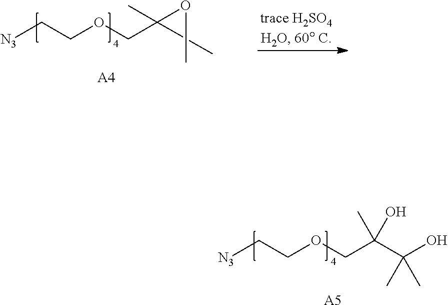

To crude A4 from the previous step, 10 mL of water and a trace amount of H₂SO₄ was added to the solution. The solution was heated to 60 degrees and left to react overnight. Water was then removed by rotary evaporation and the product was purified by column chromatography (0% to 3% MeOH in DCM) to obtain A5 (910 mg, 2.7 mmol) in 76% yield over 2 steps. HRMS-DART: Calcd for C₄H₃₀N₃O₆: m/z=336.2129 [M+H]⁺; Found: 336.2228. ¹H NMR (500 MHz, CDCl₃) δ (ppm) 3.74 (d. J=10.0 Hz. H), 3.68-3.64 (overlap, 13H), 3.46 (t. J=10.5 Hz, 2H), 3.39 (t, J=4.5 Hz, 2H), 1.24 (s, 3H), 1.19 (s, 3H), 1.07 (s, 3H).¹³C NMR (125 MHz, CDCl₃) δ (ppm) 129.2, 128.3, 77.9, 75.3, 75.0, 71.1, 70.8, 70.8, 70.7, 70.3, 70.1, 50.8, 25.4, 24.3, 20.4.

Synthesis of A6

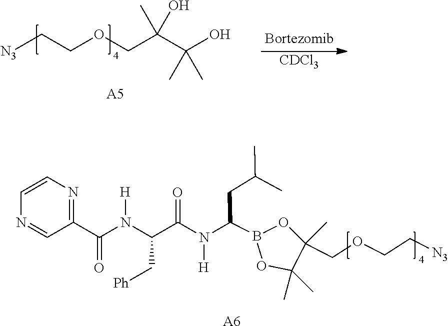

To a solution of A5 (200 mg, 0.60 mmol) dissolved in 6 mL of CDCl₃ was added bortezomib (Btz) (253 mg, 0.66 mmol). The reaction was then left to stir overnight and completion of the reaction was confirmed by ¹H NMR. CDCl₃ was evaporated under reduced pressure and CHCl₃ with ethanol as a stabilizer was added and the solution was filtered with a syringe filter. The product A6 was purified by preparatory GPC in 70% yield (287 mg, 0.42 mmol).¹H NMR (500 MHz, CDCl₃) δ (ppm) 9.35, 8.76, 8.54, 8.33, 7.30, 7.24, 6.08, 6.04, 4.83, 3.69-3.58, 3.51, 3.45, 3.43, 3.39, 3.24-3.16, 3.09-3.02, 1.43, 1.35, 1.31, 1.29, 1.27, 0.85, 0.84, 0.83, 0.82. ¹³C NMR (125 MHz, CDCl₃) δ (ppm) 171, 163, 148, 144, 144, 143, 136, 129, 128, 127.84, 83, 78, 76, 71, 71, 70, 70, 70, 53, 51, 40, 38, 37, 26, 25, 24, 23, 22, 20, 20.

Alternative Synthesis of A6

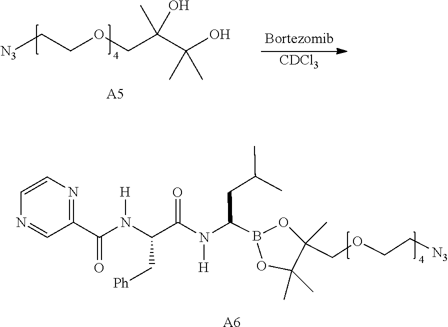

To a solution of A5 (200 mg, 0.60 mmol) in CDCl₃ (6 mL). Btz (253 mg, 0.66 mmol) was added. The reaction was then left to stir overnight and its completion was confirmed by ¹H NMR. The CDCl₃ solution was then concentrated under vacuum, redissolved in CHCl₃, filtered through a 0.45 μm filter (Nalgene), and subjected to recycling preparative HPLC. The fractions containing the pure product were collected, concentrated under vacuum and dried overnight, affording A6 (287 mg, 0.42 mmol, 70% yield). HRMS-DART: Calcd for C₁₄H₃₀N₃O₆: m/z=684.3887 [M+H]⁺; Found: 684.4183. ¹H NMR (500 MHz, CDCl₃) δ (ppm) 9.35 (s, 1H), 8.75 (overlap, 1H), 8.54 (overlap, 1H), 8.34-8.31 (overlap, 1H), 7.30-7.22 (overlap, 6H), 6.06-6.03 (overlap, 1H), 4.81 (q. J=8.5 Hz), 3.68-3.57 (overlap, 14H), 3.52-3.49 (overlap, 1H), 3.45-3.42 (overlap, 1H), 3.38 (t, J=5.0 Hz, 2H), 3.24-3.15 (overlap, 2H), 3.09-3.01 (overlap, 1H), 1.42 (sept, J=6.5 Hz, 1H), 1.34 (t, J=7.5 Hz, 2H), 1.38 (s, 3H), 1.28 (s, 3H), 1.27 (s, 3H), 0.85-0.81 (overlap 6H). ³C NMR (125 MHz, CDCl₃) δ (ppm) 171.2, 163.0, 147.6, 144.4, 144.1, 144.1, 142.9, 136.5, 129.6, 128.8, 128.8, 127.2, 127.2, 83.80, 83.7, 83.2, 83.1, 77.4, 75.6, 75.6, 71.0, 70.9, 70.8, 70.8, 70.6, 70.6, 70.2, 53.8, 50.8, 39.9, 38.5, 38.4, 25.8, 25.6, 24.3, 24.3, 23.2, 22.0, 20.8, 20.7.

Synthesis of Btz-MM

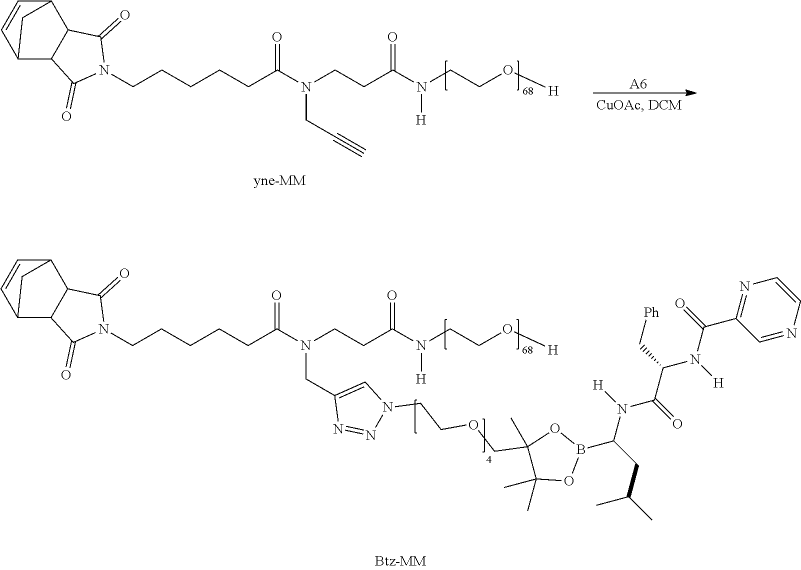

To a vial, yne-MM (513.3 mg, 0.152 mmol, 1.0 eq), A6 (125.0 mg, 0.183 mmol, 1.20 eq), and DCM (19.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the pure product as a solid (504.3 mg, 82% yield). ¹H NMR (0.500 MHz, CDCl₃) δ (ppm) 9.31, 8.73, 8.51, 8.33-8.28, 7.21, 6.57, 6.45, 6.26-6.16, 4.81, 4.59, 4.51, 4.47, 3.84-3.78, 3.71-3.34, 3.24, 3.17, 3.01, 2.64, 2.54, 2.45, 2.34, 2.26, 1.65-1.37, 1.36-1.18, 0.80.

Synthesis of Btz-BBP

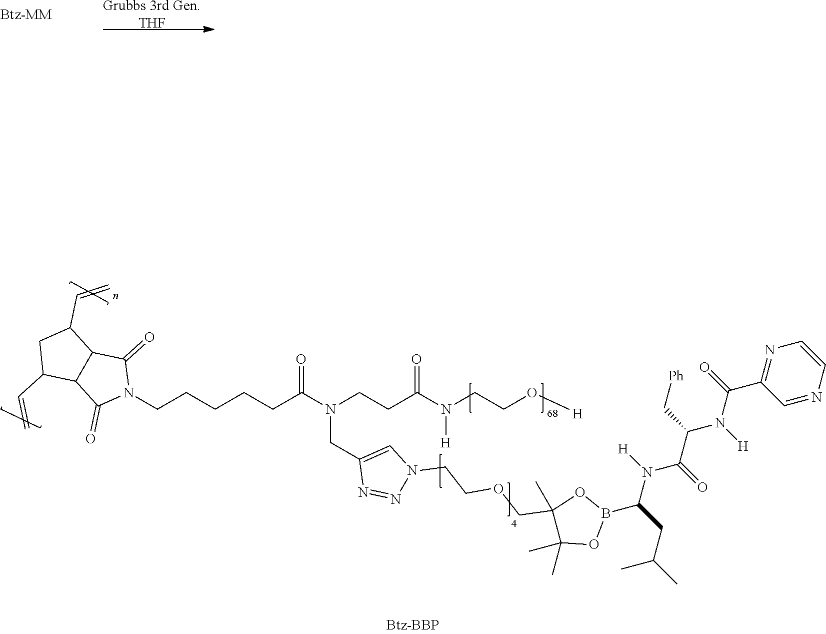

To a vial containing a stir bar. Btz-MM (80.5 mg, 19.9 μmol, 10.0 eq) was added. To another vial, a solution of 3^{rd} generation Grubbs catalyst (Ru, 0.02 M in THF) was freshly prepared. THF (397.3 μL) was then added to the vial containing Btz-MM, followed by the addition of Ru solution (99.3 μL, 1.98 μmol, 1.0 eq) to give the desired DP of 10, while achieving a total Btz-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL x 3, solvent exchange every 6 h). The dialyzed solution of Btz-BBP was then concentrated to the desired concentration via centrifugation with a filter tube. Alternatively, Btz-BBP can also be acquired by lyophilization. A mass spectrum using MALDI appears in FIG. 2 and a resulting GPC trace appears in FIG. 3 for Btz-BBP.

Synthesis of A7

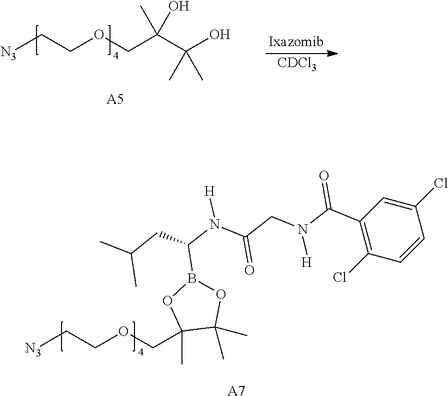

To a solution of A5 (20 mg, 0.06 mmol) dissolved in 0.6 mL of CDCl₃ was added ixazomib (Ixa) (25 mg, 0.069 mmol). The reaction was then left to stir overnight and completion of the reaction was confirmed by H NMR. CDCl₃ was evaporated under reduced pressure and CHCl₃ with ethanol as a stabilizer was added and the solution was filtered with a syringe filter. The product A6 was purified by preparatory GPC in 67% yield (32 mg, 0.040 mmol).¹H NMR (500 MHz, CDCl₃) δ (ppm) 7.64, 7.35, 6.60, 6.54, 4.21-4.10, 3.67-3.54, 3.48-3.42, 3.38, 3.28-3.21, 1.62, 1.48-1.40, 1.29, 1.26, 1.23, 0.92, 0.91, 0.91, 0.90.

Synthesis of Ixa-MM

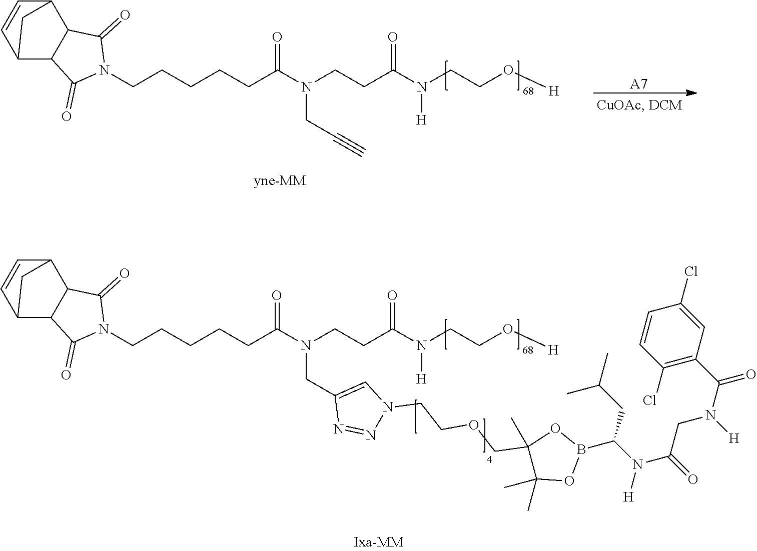

To a vial, yne-MM (170.5 mg, 0.051 mmol, 1.0 eq), A7 (41.0 mg, 0.058 mmol, 1.15 eq), and DCM (5.0 mL) were added. CuOAc (a pinch) was then added and the reaction mixture was stirred under N₂ atmosphere. The reaction was complete in -1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the pure product as a solid (185.5 mg, 90% yield). ¹H NMR (500 MHz, CDCl₃) δ (ppm) 7.71, 7.68, 7.62, 7.44-7.38, 7.33, 6.94-6.90, 6.83, 6.70, 6.57, 6.26, 4.58, 4.51, 4.47, 4.20-4.10, 3.84, 3.74-3.33, 3.23, 3.13, 2.68, 2.64, 2.53, 2.44, 2.33, 2.27, 1.63-1.48, 1.40, 1.31-1.16, 0.88.

Synthesis of Ixa-BBP

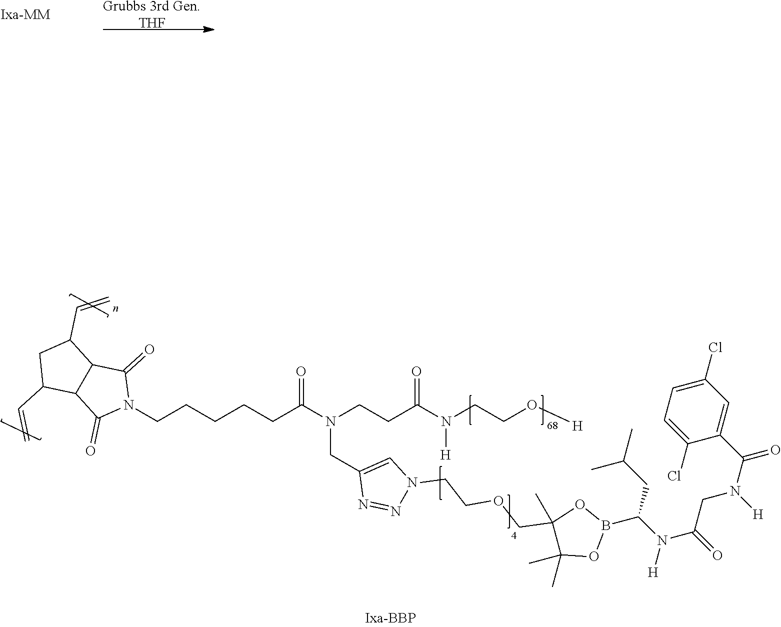

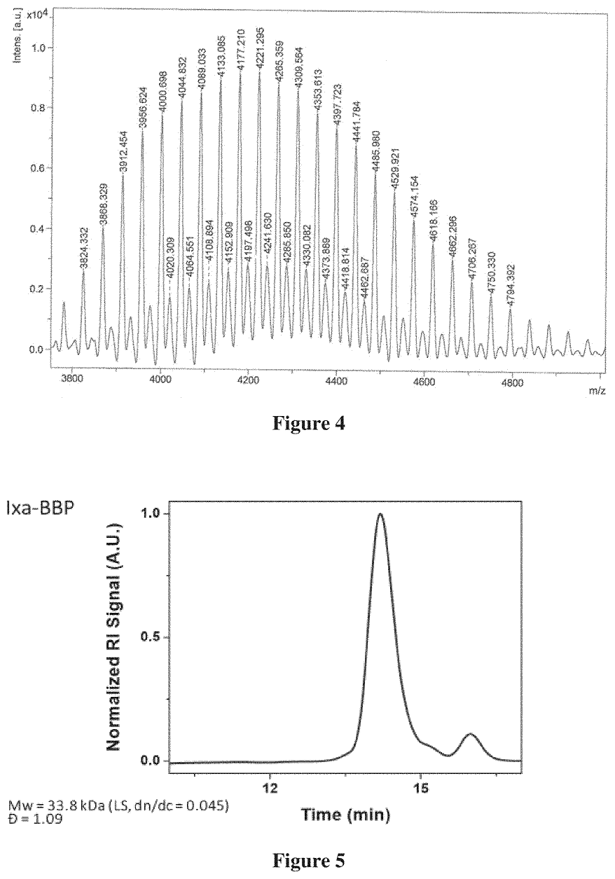

Ixa-BBP was synthesized in a similar manner to Btz-BBP. A mass spectrum using MALDI appears in FIG. 4 and a resulting GPC trace appears in FIG. 5 for Ixa-BBP.

Synthesis of B1

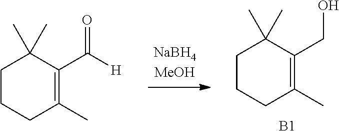

To an ice bath cooled solution of β-Cyclocitral (1.0 g, 6.6 mmol) in MeOH (6 mL) was added NaBH₄ (249 mg, 6.6 mmol). The reaction was stirred and allowed to warm up to room temperature. After 1 hour. EtOAc was added and the solution was extracted 3 times with H₂O. The organic layer was dried over Na₂SO₄ and concentrated under reduced pressure resulting in B1 (955 mg, 6.2 mmol, 94%) which was used without further purification. ¹H NMR (500 MHz, CDCl₃) δ (ppm) 4.14, 1.98, 1.75, 1.60, 1.46, 1.04.

Synthesis of B2

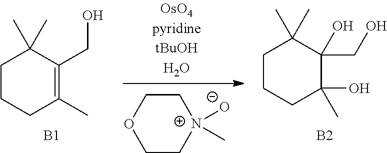

B1 (900 mg, 5.8 mmol) was dissolved in a mixture of tBuOH (10 mL), pyridine (1 mL), and H₂O (1.5 mL). OsO₄ (30 mg, 0.12 mmol) and (814 mg, 7.0 mmol) were then added to the solution. The solution was stirred and warmed to 55° C., and left to react for one day. The reaction was cooled and 4 mL of a 20% aqueous sodium bisulfite solution was added and the solution was allowed to stir for half an hour. EtOAc was added and the reaction was extracted with brine twice. The organic layer was dried over Na₂SO₄ and concentrated under reduced pressure. The crude mixture was then purified by column chromatography to yield B2 (580 mg, 3.1 mmol) in 53% yield. ¹H NMR (500 MHz, CDCl₃) δ (ppm) 4.08, 3.79, 1.79-1.74, 1.66-1.47, 1.22, 1.05, 0.97.

Synthesis of the B3

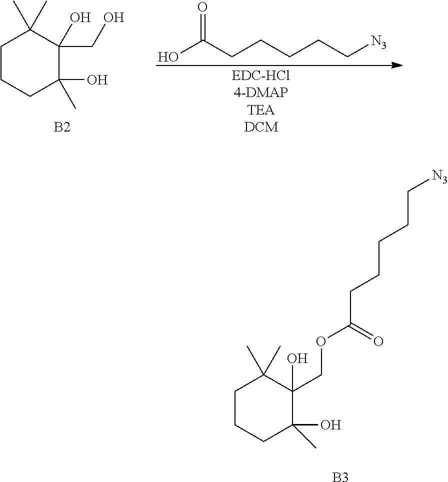

B2 (400 mg, 2.1 mmol), 6-azidohexanoic acid (330 mg, 2.1 mmol), 4-DMAP (134 mg, 1.1 mmol), and TEA (303 mg, 3.0 mmol) were dissolved in 10 mL of DCM and the solution was cooled in an ice bath. EDC-HCl (455 mg, 2.3 mmol) was added to the solution and the mixture was left to stir overnight. The solution was concentrated under reduced pressure and the crude mixture was then purified by column chromatography to obtain B3 (0.589 mg, 1.8 mmol) in 86% yield. ¹H NMR (500 MHz, CDCl₃) δ (ppm) 4.41, 4.34, 3.31, 3.30, 3.28, 3.27, 2.64, 2.38, 1.80-1.47, 1.46-1.41, 1.38, 1.13-1.06, 1.02.

Synthesis of B4

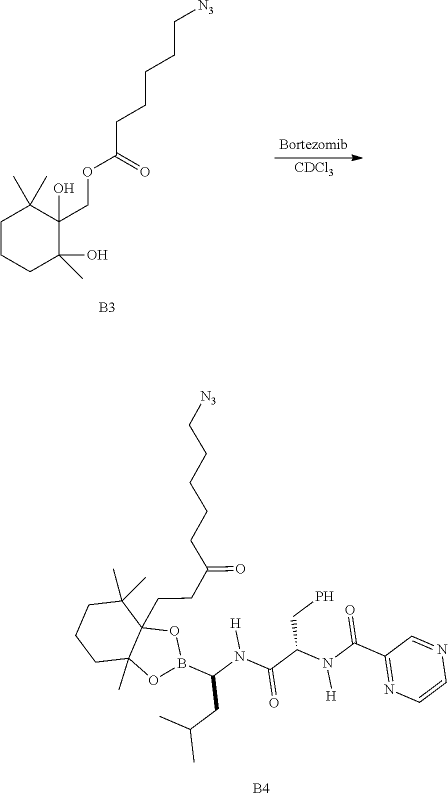

B3 (300 mg, 0.90 mmol) and Bortezomib (384 mg, 1.0 mmol) were dissolved in 5 mL of CDCl₃. The solution was heated to 45° C., and left to stir overnight. Completion of the reaction was confirmed by ¹H NMR. CDCl₃ was evaporated under reduced pressure and CHCl₃ with ethanol as a stabilizer was added and the solution was filtered with a syringe filter. The product B4 was purified by preparatory GPC in 95% yield (580 mg, 0.86 mmol).¹H NMR (500 MHz, CDCl₃) δ (ppm) 9.35, 8.75, 8.53, 8.32, 7.30, 7.22, 6.00, 5.95, 4.81, 4.30, 4.28, 4.17, 4.08, 3.35, 3.26, 3.20, 2.31, 1.82, 1.71-1.33, 1.33-1.26, 1.16, 1.08, 0.98, 0.96, 0.85-0.81.

Synthesis of BtzC-MM

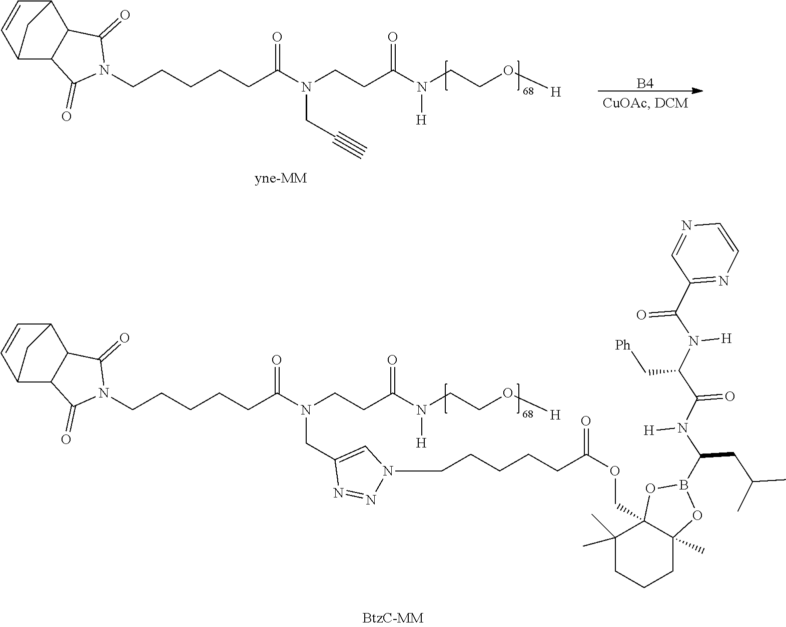

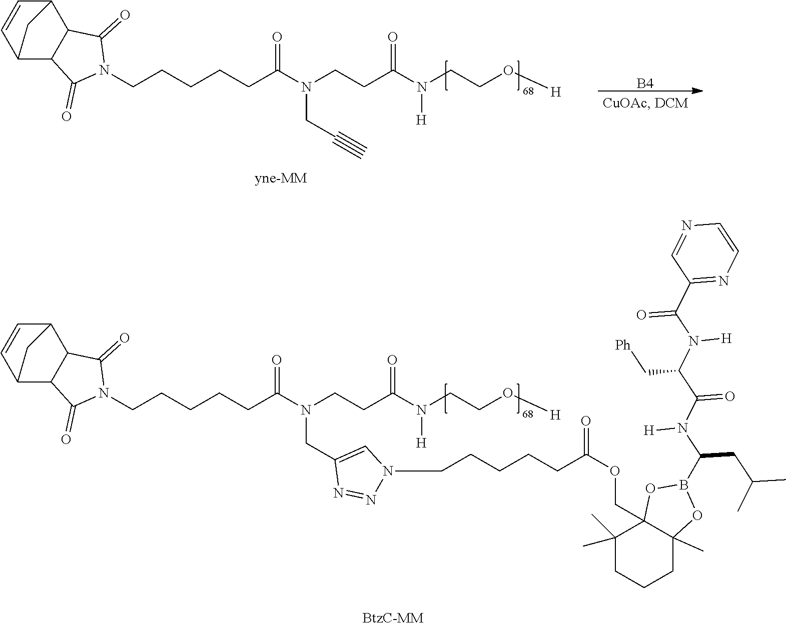

To a vial, yne-MM (204.7 mg, 0.061 mmol, 1.0 eq), B4 (48.2 mg, 0.070 mmol, 1.15 eq), and DCM (12.0 mL) were added. CuOAc (a pinch) was then added and the reaction mixture was stirred under N₂ atmosphere. The reaction was complete in -1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the pure product as a solid (232.2 mg, 94% yield). ¹H NMR (500 MHz, CDCl₃) δ (ppm) 9.30, 8.72, 8.50, 8.30, 7.55, 7.49, 7.21, 6.53, 6.41, 6.31-6.21, 4.82, 4.57, 4.49, 4.31-4.24, 4.13, 4.04, 3.78, 3.69-3.38, 3.33, 3.22, 3.15, 2.63, 2.59, 2.52, 2.48, 2.39, 2.30, 2.25, 1.88-1.46, 1.40-1.12, 1.05, 0.94, 0.92, 0.79.

Synthesis of BtzC-BBP

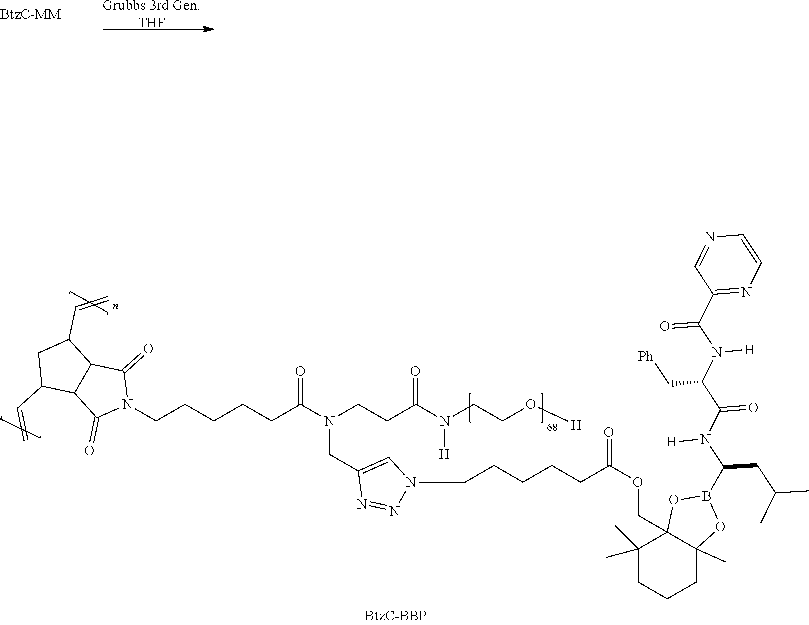

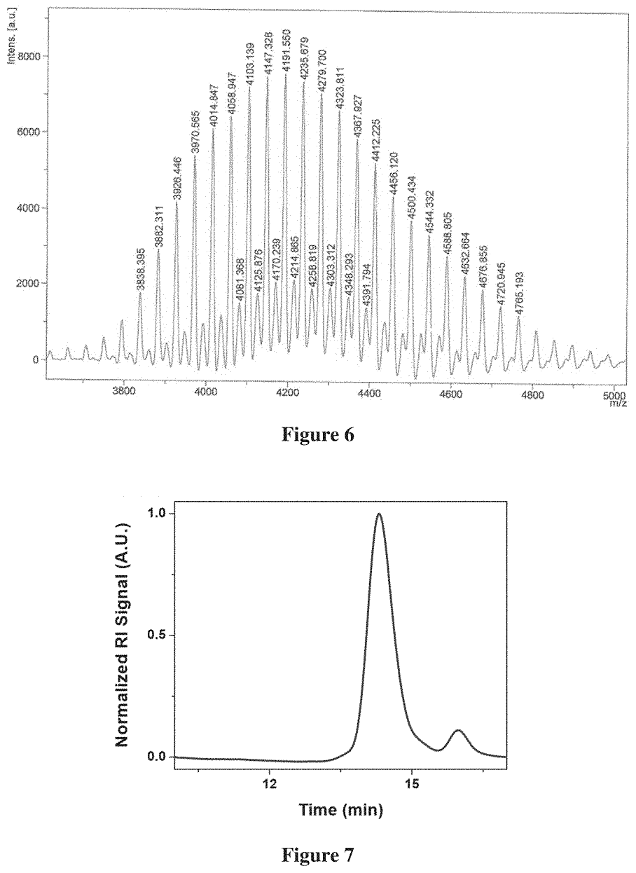

BtzC-BBP was synthesized in a similar manner to Btz-BBP. A mass spectrum using MALDI appears in FIG. 6 and a resulting GPC trace appears in FIG. 7 for BtzC-BBP.

### In Vitro and In Vivo Testing

After synthesis, Btz-BBP was tested in a variety of biological experiments in order to study its toxicity and therapeutic efficacy. Toxicological studies were carried out including in vitro cell viability experiments and in vivo survivability studies (FIGS. 8A to 8B and FIG. 9A, respectively). The in vitro cell viability studies were carried out in both MM S model (for experimental details see Manier, S, et al., Science Translational Medicine 2017, 9, 389) and KMS11 flank cancer model. These studies showed that the BBP not loaded with Btz proved to be safe with 100% cell viability even at greater than 10⁵ nM (FIGS. 8A and 8B). Additionally, when Btz was loaded onto the BBP. Btz-BBP resulted in higher cell viability compared to the free Btz (FIGS. 8A and 8B). For the maximum tolerated dose (MTD) studies, animals (n=3-5) were dosed 4 times, with the exception that the 20 mg/kg group was dosed three times. This toxicity study established 0.75 mg/kg as the maximum tolerated dose for free Btz (FIG. 9A). Moreover, Btz-BBP showed completely healthy animals even at >25-fold the MTD, or 20-fold higher than 1 mg/kg. This suggests a much wider therapeutic index, as well as a much more stable Btz prodrug compared to prior examples.

Therapeutic efficacy studies were also undertaken. Studies treating SCIB KMS11 and MM1S subcutaneous mouse models with dosing once per week showed the therapeutic efficacy of Btz-BBP (FIGS. 10C to 11B). These studies showed that Btz-BBP performed comparably to free Btz at the MTD (0.75 mg/kg), suggesting efficient cleavage of the boronate linker, releasing free Btz at tumor sites. Furthermore, an equivalent 18.75 mg/kg Btz (25-fold the free Btz MTD) could be dosed when utilizing Btz-BBP, resulting in dramatic enhancements in efficacy (FIGS. 11A and 11B). Imaging of tumor size was monitored by bioluminescence and showed that mice treated with Btz-BBP had smaller tumors compared to those dosed with Btz (imaging at day 20 in FIGS. 12A to 12C).

### Synthetic Procedures: Radical Polymerization

Synthesis of C₁

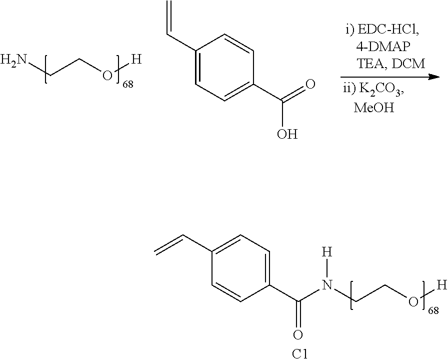

Vinylbenzoic acid (400 mg, 2.67 mmol) and NH₂PEG3kOH (2 g, 0.67 mmol) were dissolved in 5 mL of DCM. EDC-HCl (530 mg, 2.67 mmol), 4-DMAP (158 mg, 1.3 mmol), and TEA (370 μL, 2.67 mmol) were added to the solution. The reaction was left to stir overnight. After completion. DCM was removed under reduced pressure and K₂CO₃ (690 mg, 5 mmol) and MeOH (20 mL) were added. The solution was heated to 50° C., and left to stir for 3 hours. MeOH was removed under reduced pressure. DCM (100 mL) was added to the mixture and the organic solution was extracted with 100 mL of 50% brine two times. The organic layer was dried with Na₂SO₄ and concentrated under reduced pressure. The crude product was purified with preparatory GPC to obtain C1 in 70% yield (1.4 g, 0.46 mmol). ¹H NMR (500 MHz, CDCl₃) δ (ppm) 7.78, 7.77, 7.46, 7.45, 6.83, 6.76, 6.74, 6.73, 6.71, 5.84, 5.81, 5.35, 5.34, 3.77, 3.76, 3.75, 3.75, 3.73, 3.72, 3.72, 3.69, 3.68, 3.68, 3.68, 3.67, 3.67, 3.66, 3.66, 3.65, 3.64, 3.64, 3.63, 3.62, 3.62, 3.61, 3.60, 3.53, 3.52, 3.52, 3.51.

Synthesis of Alternating Styrene-Co-Maleimide Polymers C₂ and C₃

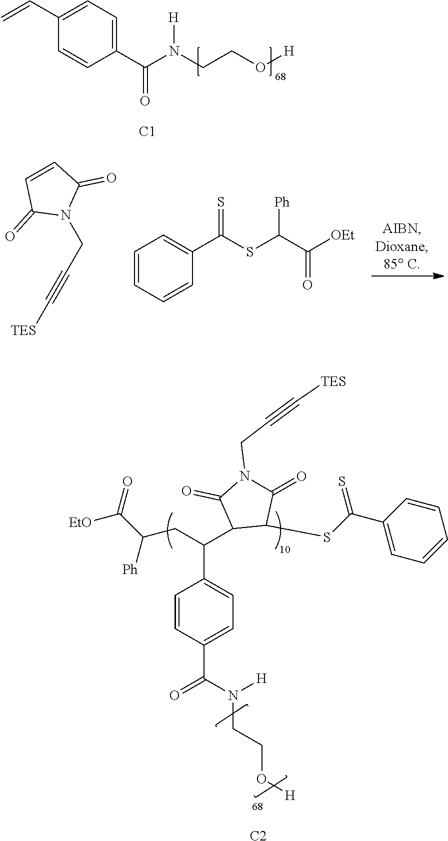

C1 (350 mg, 0.117 mmol),1-(3-(triethylsilyl)prop-2-ynyl)-1Hpyrrole-2,5-dione (29 mg, 0.117 mmol), Ethyl 2-(phenylcarbonothioylthio)-2-phenylacetate (3 mg, 0.0097 mmol), and Azobisisobutyronitrile (0.7 mg, 0.0042 mmol) were dissolved in 600 μL of 1,4-Dioxane. The solution was added to a 5 mL ampule and put under 3 freeze-pump-thaw cycles afterwhich the ampule was flame sealed under vacuum. The reaction was heated to 85° C., and left to react for 48 hours. The solution was then dissolved in chloroform and the polymer was purified with a preparatory GPC to obtain C2 in 82% yield (290 mg, —0.0085 mmol). ¹H NMR (500 MHz, CDCl₃) δ (ppm) 7.75-6.25 (broad), 4.31-4.00 (broad), 3.78, 3.77, 3.76, 3.73, 3.72, 3.72, 3.71, 3.70, 3.70, 3.68, 3.67, 3.66, 3.65, 3.64, 3.63, 3.63, 3.62, 3.60, 3.60, 3.60, 3.59, 3.50, 3.49, 3.48, 1.94-1.55 (broad), 0.97, 0.97, 0.97, 0.96, 0.96, 0.95, 0.95, 0.94, 0.93, 0.93, 0.91, 0.90, 0.60, 0.59, 0.58, 0.58, 0.57, 0.57, 0.56, 0.55, 0.55, 0.54, 0.53.

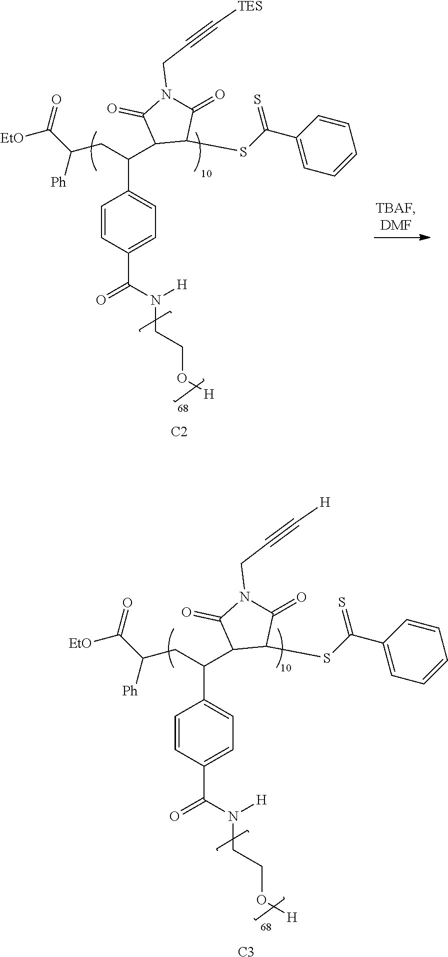

C2 (200 mg, 0.0059 mmol) was dissolved in 2 mL of DMF and TBAF (100 μL, 1M in THF) was added dropwise. The reaction was stirred for 1 hour after which chloroform was added and the polymer was purified with preparatory GPC to obtain C3 in 91% yield (172 mg, 0.0054 mmol). ¹H NMR (500 MHz, CDCl₃) δ (ppm) 7.75-6.25 (broad), 4.31-4.00 (broad), 3.78, 3.77, 3.76, 3.73, 3.72, 3.72, 3.71, 3.70, 3.70, 3.68, 3.67, 3.66, 3.65, 3.64, 3.63, 3.63, 3.62, 3.60, 3.60, 3.60, 3.59, 3.50, 3.49, 3.48, 2.55-2.34 (broad), 1.94-1.55 (broad).

Synthesis of C₄ (C₃ polymer loaded with Bortezomib)

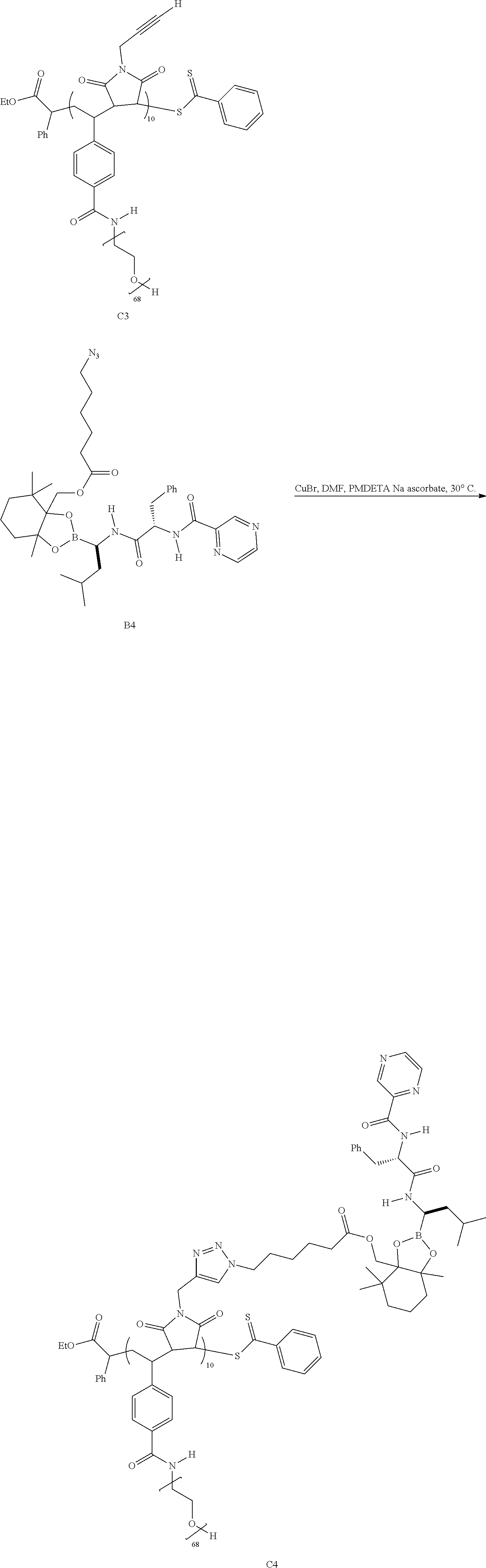

C3 (100 mg, 0.0059 mmol), B4 (200 mg, 0.3 mmol), CuBr (43 mg, 0.3 mmol), PMDETA (78 mg, 0.45 mmol), and Na ascorbate (120 mg, 0.6 mmol) were dissolved in 5 mL DMF. The reaction was heated to 30° C., and left to react for 1 day. The reaction mixture was put into 6k MWCO regenerated cellulose dialysis tubing and dialyzed against MeOH and H₂O after which the solution in the dialysis tubing was concentrated under reduced pressure to obtain C4 as an off-white solid in 69% yield (160 mg, 0.0041 mmol). ¹H NMR (500 MHz, CDCl₃) δ (ppm) 9.36, 8.79, 8.54, 8.32, 7.79, 7.78, 7.75-6.25 (broad), 7.38, 7.37, 7.01, 7.00, 6.82, 6.77, 6.75, 6.74, 6.72, 4.91, 4.84, 4.81, 4.33, 4.33, 4.32, 4.29, 4.26, 3.78, 3.77, 3.76, 3.73, 3.72, 3.72, 3.71, 3.70, 3.70, 3.68, 3.67, 3.66, 3.65, 3.64, 3.63, 3.63, 3.62, 3.60, 3.60, 3.60, 3.59, 3.50, 3.49, 3.48, 3.32, 3.20, 2.12, 2.12, 2.08, 2.05, 2.05, 2.02, 1.99, 1.81, 1.78, 1.76, 1.68, 1.62, 1.57, 1.56, 1.49, 1.48, 1.47, 1.43, 1.40, 1.39, 1.37, 1.36, 1.35, 1.32, 1.31, 1.30, 1.29, 1.28, 1.26, 1.26, 1.25, 1.25, 1.24, 1.23, 1.22, 1.22, 1.21, 1.18, 1.17, 1.16, 1.16, 1.15, 1.14, 1.13, 1.12, 1.11, 1.10, 1.09, 1.04, 1.03, 1.02, 0.98, 0.97, 0.96, 0.95, 0.93, 0.91, 0.90, 0.90, 0.83.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### REFERENCES

1) Palumbo, A.; Anderson. K. Multiple myeloma. N. Engl. J. Med, 2011, 364, 1046-1060.
2) Rollig, C.; Knop, S.; Bornhauser. M. Multiple myeloma. Lancet 2015, 385, 2197-2208.
3) Ghobrial, I. M.; Detappe, A.; Anderson, K. C.; Steensma. D. P. The bone-marrow niche in MDS and MGUS: implications for AML and MM. Nat. Rev. Clin. Oncol, 2018, 15, 219-233.
4) Bustoros, M.; Mouhieddine, T. H.; Detappe, A.; Ghobrial, I. M. Established and novel prognostic biomarkers in multiple myeloma. Am Soc Clin Oncol Educ Book 2017, 37.548-560.
5) Attal. M.; Lauwers-Cances, V.; Hulin. C.; Leleu, X.; Caillot. D.; Escoffre, M.; Arnulf, B.; Macro, M.; Belhadj, K.; Garderet. L.; Roussel, M.; Payen, C.; Mathiot, C.; Fermand, J. P.; Meuleman, N.; Rollet, S.; Maglio. M. E.; Zeytoonjian. A. A.; Weller. E. A.; Munshi, N.; Anderson, K. C.; Richardson, P. G.; Facon, T.; Avet-Loiseau, H.; Harousseau, J. L.; Moreau, P.; Study, I. F. M. Lenalidomide, bortezomib, and dexamethasone with transplantation for myeloma. N. Engl. J. Med, 2017, 376, 1311-1320.
6) Nooka, A. K.; Kaufman. J. L.; Muppidi, S.; Langston, A. Heffner, L. T.; Gleason, C.; Casbourne, D.; Saxe. D.; Boise, L. H.; Lonial, S. Consolidation and maintenance therapy with lenalidomide, bortezomib and dexamethasone (RVD) in high-risk myeloma patients. Leukemia 2014, 28, 690-693.
7) Tsvetkov, P.; Detappe, A.; Cai, K.; Keys. H. R.; Brune. Z.; Ying, W.; Thiru, P.; Reidy, M.. Kugener, G.; Rossen. J.; Kocak, M.; Kory. N.; Tshcmiak, A. Santagata, S.; Whitesell, L.; Ghobrial. I. M.; Markley, J. L.; Lindquist. S.; Golub. T. R. Mitochondrial metabolism promotes adaptation to proteotoxic stress. Nat. Chem. Biol, 2019, 15, 681-689.
8) Manasanch, E. E.; Orlowski, R. Z. Proteasome inhibitors in cancer therapy. Nat. Rev. Clin. Oncol, 2017, 14, 417-433.
9) Kisselev, A. F.; Goldberg. A. L. Proteasome inhibitors: from research tools to drug candidates. Chem. Biol, 2001, 8, 739-758.
10) Richardson, P. G.; Hideshima. T.; Anderson. K. C. Bortezomib (PS-341): a novel, first-in-class proteasome inhibitor for the treatment of multiple myeloma and other cancers. Cancer Control 2003, 10, 361-369.
11) San Miguel, J.; Blade, J.; Boccadoro. M.; Cavenagh, J.; Glasmacher. A.; Jagannath, S.; Lonial, S.: Orlowski, R. Z.; Sonneveld. P.; Ludwig, H. A practical update on the use of bortezomib in the management of multiple myeloma. Oncologist 2006, 11, 51-61.
12) Demo. S. D.; Kirk, C. J.; Aujay, M. A.; Buchholz. T. J.; Dajee, M.: Ho. M. N. Jiang, J.; Laidig. G. J.; Lewis, E. R.; Parlati, F.: Shenk. K. D.: Smyth, M. S.; Sun. C. M. Vallone, M. K.; Woo, T. M.; Molineaux, C. J.; Bennett, M. K., Antitumor activity of PR-171, a novel irreversible inhibitor of the proteasome. Cancer Res, 2007, 67, 6383-6391.
13) Kim. S. H.: Kramer, I. Physicochemical stability of carfilzomib (Kyprolis®) containing solutions in glass vials, ready-to-administer plastic syringes and infusion bags over a 28-day storage period. J. Oncol. Pharm. Pract, 2017, 339-350.
14) Gavazzoni. M.; Vizzardi. E.; Gorga, E.; Bonadei. I.; Rossi. L.; Belotti. A.; Rossi, G.; Ribolla, R.; Metra. M.; Raddino, R., Mechanism of cardiovascular toxicity by proteasome inhibitors: New paradigm derived from clinical and pre-clinical evidence. Eur. J. Pharmacol, 2018, 828, 80-88.
15) lannaccone, A.; Bruno, G.; Ravera, A.; Gay. F.; Salvini, M.; Bringhen. S.; Sabia, L.; Avenatti, E.; Veglio, F.; Milan. A. Evaluation of cardiovascular toxicity associated with treatments containing proteasome inhibitors in multiple myeloma therapy. High Blood Press. Cardiovasc. Prev. 2018.25, 209-218.
16) Swami. A.; Reagan. M. R.; Basto, P.; Mishima. Y.; Kamaly. N.; Glavey, S.; Zhang. S.; Moschetta, M.; Seevaratnam, D.; Zhang, Y.; Liu, J.; Memarzadeh, M.; Wu, J.; Manier, S.; Shi, J.; Bertrand, N.; Lu, Z. N.; Nagano, K.; Baron, R.; Sacco, A.; Roccaro. A. M.; Farokhzad, O. C.; Ghobrial, I. M. Engineered nanomedicine for myeloma and bone microenvironment targeting. Proc. Natl. Acad. Sci. U.S.A. 2014, 111, 10287-10292.
17) Shen. S.; Du. X. J.; Liu, J.; Sun. R.; Zhu. Y. H.; Wang. J. Delivery of bortezomib with nanoparticles for basal-like triple-negative breast cancer therapy. J. Control. Release 2015, 208, 14-24.
18) Thamake, S. I.; Raut, S. L.; Gryczynski, Z.; Ranjan, A. P.; Vishwanatha, J. K. Alendronate coated poly-lactic-co-glycolic acid (PLGA) nanoparticles for active targeting of metastatic breast cancer. Biomaterials 2012, 7164-7173.
19) Frasco, M. F.; Almeida, G. M.; Santos-Silva. F.; Pereira Mdo C.: Coelho, M. A. Transferrin surface-modified PLGA nanoparticles-mediated delivery of a proteasome inhibitor to human pancreatic cancer cells. J. Biomed. Mater. Res. A 2015, 1476-1484.
20) Shen, J.; Song. G.; An, M.; Li, X.; Wu. N.; Ruan, K.: Hu, J.; Hu, R. The use of hollow mesoporous silica nanospheres to encapsulate bortezomib and improve efficacy for non-small cell lung cancer therapy. Biomaterials 2014, 35, 316-326.
21) Zuccari, G.; Milelli, A.; Pastorino, F.; Loi, M.; Petretto, A.; Parise, A.; Marchetti, C.; Minarini, A.; Cilli, M.; Emionite, L.: Di Paolo, D.; Brignole, C.; Piaggio. F.; Perri, P.; Tumiatti, V.; Pistoia, V.; Pagnan, G.; Ponzoni, M. Tumor vascular targeted liposomal-bortezomib minimizes side effects and increases therapeutic activity in human neuroblastoma. J Control. Release 2015, 211, 44-52.
22) Ashley, J. D.; Stefanick. J. F.; Schroeder, V. A.; Suckow, M. A.; Kiziltepe, T.; Bilgicer, B. Liposomal bortezomib nanoparticles via boronic ester prodrug formulation for improved therapeutic efficacy in vivo. J. Med. Chem, 2014, 57, 5282-5292.
23) Wu. K.; Cheng. R.; Zhang, J.; Meng, F.; Deng, C.; Zhong, Z. Micellar nanoformulation of lipophilizcd bortezomib: high drug loading, improved tolerability and targeted treatment of triple negative breast cancer. J. Mater. Chem. B 2017, S, 5658-5667.
24) Su. J.; Chen. F.; Cryns, V. L.; Messersmith, P. B. Catechol polymers for pH-responsive, targeted drug delivery to cancer cells. J. Am. Chem. Soc. 2011, 133, 11850-11853.
25) Xu. W.; Ding, J.; Li, L.; Xiao, C.; Zhuang, X.; Chen, X. Acid-labile boronate-bridged dextran-bortezomib conjugate with up-regulated hypoxic tumor suppression. Chem. Commun. 2015, 51, 6812-6815.
26) Lu, X.; Chai. Z.; Lu, L.; Ruan, H.; Wang. R.; Zhan. C.; Xie, C.; Pan. J.; Liu. M.; Wang, H.; Lu, W. Bortezomib dendrimer prodrug-based nanoparticle system. Adv. Funct. Mater, 2019, 29, 1807941.
27) Wu. S.; Qi, R.; Kuang, H.; Wei, Y.; Jing, X.; Meng, F.; Huang, Y, pH-responsive drug delivery by amphiphilic copolymer through boronate-catechol complexation. Chem Plus Chem 2013, 78, 175-184.
28) Min, J.; Moon, H.; Yang, H. J.; Shin, H. H.; Hong, S. Y.; Kang, S. Development of P22 viral capsid nanocomposites as anti-cancer drug, bortezomib (BTZ), delivery nanoplatforms. Macromol. Biosci. 2014, 14, 557-564.
29) Zhu. J.; Huo. Q.; Xu, M.; Yang, F.; Li, Y.; Shi, H.; Niu, Y.; Liu, Y. Bortezomib-catechol conjugated prodrug micelles: combining bone targeting and aryl boronate-based pH-responsive drug release for cancer bone-metastasis therapy. Nanoscale 2018, 10, 18387-18397.
30) Detappe, A.; Bustoros, M.; Mouhieddine, T. H.; Ghoroghchian, P. P., Advancements in nanomedicine for multiple myeloma. Trends. Mol. Med, 2018, 24, 560-574.
31) Mura, S.; Nicolas, J.; Couvreur, P. Stimuli-responsive nanocarriers for drug delivery. Nat. Mater, 2013, 12, 991-1003.
32) Shi, J.; Xiao, Z.; Kamaly, N.; Farokhzad, O. C. Self-assembled targeted nanoparticles: evolution of technologies and bench to bedside translation. Acc. Chem. Res, 2011, 44, 1123-1134.
33) Smith, B. R.; Gambhir. S. S. Nanomaterials for in vivo imaging. Chem. Rev, 2017, 117, 901-986.
34) Johnson, J. A.; Lu, Y. Y.; Burls. A. O.; Xia, Y.; Durrell. A. C.; Tirrell, D. A.; Grubbs. R. H. Drug-loaded, bivalent-bottle-brush polymers by graft-through ROMP. Macromolecules 2010, 43, 10326-10335.
35) Liu, J.; Burts, A. O.; Li, Y.; Zhukhovitskiy, A. Z.; Ottaviani, M. F. Turro, N. J.; Johnson, J. A. “Brush-first” method for the parallel synthesis of photocleavable, nitroxide-labeled PEG star polymers. J. Am. Chem. Soc. 2012, 134, 16337-16344.
36) Liao, L.; Liu, J.; Dreaden, E. C.; Morton, S. W.; Shopsowitz, K. E.; Hammond, P. T.; Johnson, J. A. A convergent synthetic platform for single-nanoparticle combination cancer therapy: ratiometric loading and controlled released of cisplatin, doxorubicin, and camptothecin. J. Am. Chem. Soc. 2014, 136, 5896-5899.
37) Sowers, M. A.; McCombs. J. R.; Wang, Y.; Paletta, J. T.; Morton, S. W.; Dreaden. E. C. Boska, M. D.; Ottaviani, M. F.; Hammond, P. T.; Rajca, A.; Johnson, J. A. Redox-responsive branched-bottlebrush polymers for in vivo MRI and fluorescence imaging. Nat. Commun. 2014, 5, 5460.
38) Barnes. J. C.; Bruno, P. M.; Nguyen. H. V.-T.; Liao, L.; Liu, J.; Hemann, M. T.; Johnson, J. A. Using an RNAi signature assay to guide the design of three-drug conjugated nanoparticles with validated mechanisms, in vivo efficacy, and low toxicity. J. Am. Chem. Soc. 2016, 138, 12494-12501.
39) Nguyen, H. V.-T.; Chen, Q.; Paletta, J. T.; Harvey, P.; Jiang. Y.; Zhang, H.; Boska, M. D.; Ottaviani, M. F.; Jasanoff, A. Rajca, A.; Johnson, J. A. Nitroxide-based macromolecular contrast agents with unprecedented transverse relaxivity and stability for magnetic resonance imaging of tumors. ACS Cent. Sci. 2017, 3, 800-811.
40) Golder, M. R.; Liu, J.; Andersen. J. N. Shipitsin, M. V.; Vohidov, F.; Nguyen, H. V.-T.; Ehrlich. D. C.; Huh, S. J.; Vangamudi, B.; Economides, K. D.; Neenan, A. M.; Ackley. J. C.; Baddour, J.; Paramasivan, S.; Brady. S. W.; Held, E. J.; Reiter, L. A.; Saucier-Sawyer, J. K.; Kopesky, P. W.; Chickering, D. E.; Blume-Jensen, P.; Johnson, J. A. Reduction of liver fibrosis by rationally designed macromolecular telmisartan prodrugs. Nat. Biomed. Eng. 2018, 2, 822-830.
41) Nguyen, H. V.-T.; Detappe, A.: Harvey, P.; Gallagher, N.: Mathieu, C.: Agius, M. P.: Zavidij, O.; Wang, W.; Jiang, Y., Rajca, A.; Jasanoff, A.; Ghobrial, I. M.; Ghoroghchian, P. P.; Johnson, J. A. Stimuli-responsive organic radical contrast agents for real time MRI-based tracking of prodrug activation in biological systems. Manuscript submitted for publication.
42)Nguyen, H. V.-T.; Detappe, A.: Gallagher. N. M.; Zhang, H.; Harvey. P.; Yan, C.; Mathieu, C.; Golder, M. R.; Jiang, Y.; Ottaviani, M. F.; Jasanoff, A.; Rajca, A.; Ghobrial, I. M.; Ghoroghchian, P. P.; Johnson. J. A. Triply loaded nitroxide brush-arm star polymers enable metal-free millimetric tumor detection by magnetic resonance imaging. ACS Nano 2018, 12, 11343-11354.
43) Golder, M. R.; Nguyen. H. V.-T.; Oldenhuis, N. J.; Grundler. J.; Park. E. J.; Johnson, J. A. Brush-first and ROMP-out with functional (macro)monomers: method development, structural investigations, and applications of an expanded brush-arm star polymer platform. Macromolecules 2018, 51, 9861-9870.
44) Detappe A.; Reidy, M.; Mathieu. C.; Nguyen. H. V.-T.; Coroller, T. P.; Lam. F.; Jarolim, P.; Harvey, P.; Protti. A.; Nguyen. Q. D.; Johnson, J. A.; Cremilleux. Y.; Tillement, O.; Ghobrial. I. M.; Ghoroghchian, P. P. Antibody-targeting of ultra-small nanoparticles enhances imaging sensitivity and enbales longitudinal tracking of multiple myeloma. Nanoscale 2019, 11(43): 20485-20496.
45) Nguyen. H. V.-T.; Gallagher. N. M.; Vohidov, F.; Jiang. Y.; Kawamoto. K.; Zhang, H.; Park, J. V.; Huang, Z.; Ottaviani, M. F.; Rajca, A.; Johnson, J. A. Scalable synthesis of multivalent macromonomers for ROMP. ACS Macro Lett. 2018, 7, 472-476.
46) Love, J. A.; Morgan, J. P.; Trnka, T. M.; Grubbs. R. H. A Practical and Highly Active Ruthenium-Based Catalyst that Effects the Cross Metathesis of Acrylonitrile. Angew. Chem. Int. Ed. 2002, 41, 4035-4037.
47) Chen. X.; Clennan, E. L. Reactions of an Allylic Sulfide, Sulfoxide, and Sulfone with Singlet Oxygen. The Observation of a Remarkable Diastereoselective Oxidation. J. Am. Chem. Soc. 1989, 111, 5787-5792.

1. A compound of Formula (I) or (II): 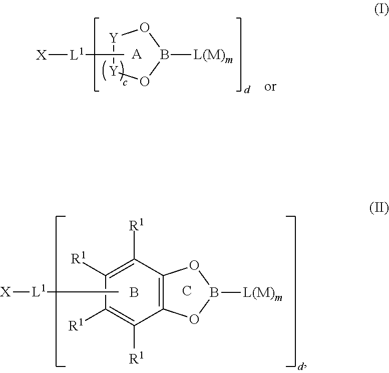
or a salt thereof, wherein:
X is a reaction handle;
L¹ is a substituted or unsubstituted linker, wherein the backbone of L¹ comprises two or more atoms;
each instance of Y is independently —C(R¹)₂—;
each instance of R is independently absent, hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or two instances of R are joined to form substituted or unsubstituted carbocyclyl or substituted or unsubstituted heterocyclyl;
each instance of R^{a} is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;
each instance of L is independently a bond or a substituted or unsubstituted linker, wherein the atom in the backbone of L attached to Ring A or Ring C is carbon;
each instance of M is independently an agent;
each instance of m is independently an integer between 1 and 10, inclusive;
each instance of c is independently an integer between 1 and 2, inclusive; and
d is an integer between 1 and 10, inclusive.

2. A polymer comprising one or more types of repeating units, wherein at least one type of the repeating units comprises a moiety of the formula: 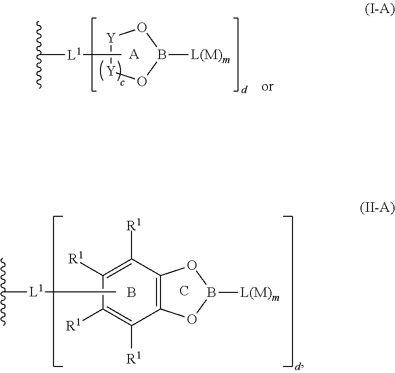
wherein:
L¹ is a substituted or unsubstituted linker, wherein the backbone of L¹ comprises two or more atoms;
each instance of Y is independently —C(R¹)₂—;
each instance of R is independently absent, hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂, or two instances of R are joined to form substituted or unsubstituted carbocyclyl or substituted or unsubstituted heterocyclyl;
each instance of R^{a} is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;
each instance of L is independently a bond or a substituted or unsubstituted linker, wherein the atom in the backbone of L attached to Ring A or Ring C is carbon;
each instance of M is independently an agent;
each instance of m is independently an integer between 1 and 10, inclusive;
each instance of c is independently an integer between 1 and 2, inclusive; and
d is an integer between 1 and 10, inclusive.

3. (canceled)

4. The compound of claim 1, or a salt thereof,
wherein X is —N₃.

5-22. (canceled)

23. The compound of claim 1, or salt thereof, wherein X is of the formula:

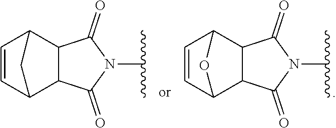

24-50. (canceled)

51. The compound of claim 1, or salt thereof, wherein

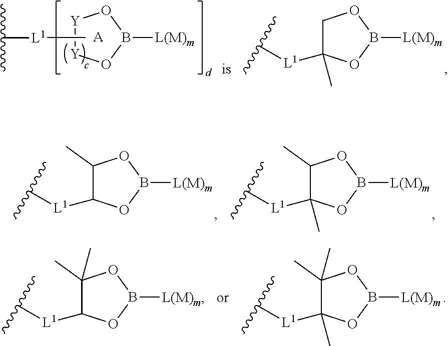

52-68. (canceled)

69. The compound of claim 1, or a salt thereof, wherein at least one instance of the agent is bortezomib.

70-73. (canceled)

74. The compound of claim 1, or salt thereof, wherein Formula I is of the formula:

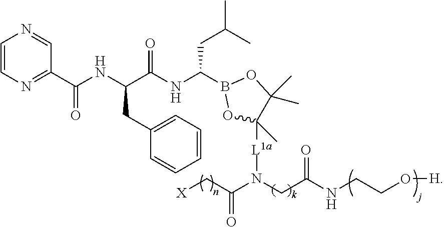

75. The compound of claim 1, or salt thereof, wherein Formula I is of the formula:

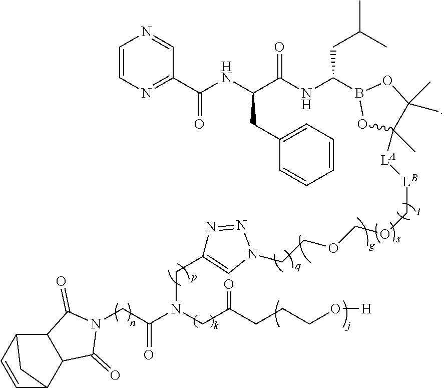

76. The compound of claim 1, or salt thereof, wherein Formula I is of the Formula I-1:

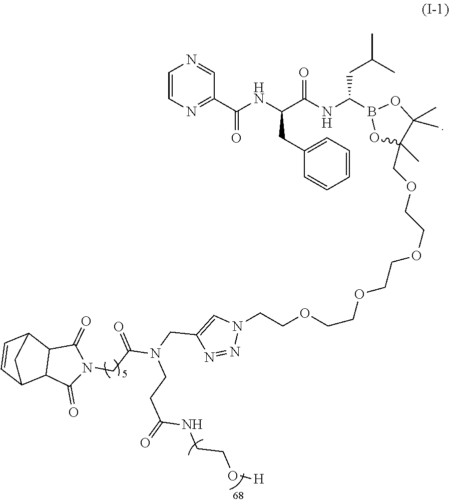

77. A method of preparing a polymer comprising polymerizing a compound claim 1, or a salt thereof, wherein X is a polymerization handle.

78-82. (canceled)

83. A method of preparing a polymer comprising reacting an existing polymer with a compound of claim 1, or salt thereof, wherein the existing polymer comprises a reaction handle able to react with X, and in the step of reacting, the reaction handle able to react with X is reacted with X.

84-85. (canceled)

86. A polymer prepared by a method of claim 77.

87. A composition comprising:
a polymer of claim 2; and
optionally an excipient.

88-89. (canceled)

90. A kit comprising:
a polymer of claim 2; and
instructions for using the polymer.

91. A method of delivering an agent to a biological sample, tissue, or cell, the method comprising contacting the biological sample, tissue, or cell with a polymer of claim 2.

92-93. (canceled)

94. A method of delivering an agent to a subject in need thereof, the method comprising administering to the subject in need thereof a polymer of claim 2.

95. (canceled)

96. A method of treating a disease in a subject in need thereof, the method comprising administering to the subject in need thereof a therapeutically effective amount of a polymer of claim 2, wherein at least one instance of M is a therapeutic agent.

97-102. (canceled)

103. A method of preventing a disease in a subject in need thereof, the method comprising administering to the subject in need thereof a therapeutically effective amount of a polymer of claim 2, wherein at least one instance of M is a prophylactic agent.

104. A method of diagnosing a disease in a subject in need thereof, the method comprising administering to the subject in need thereof a therapeutically effective amount of a polymer of claim 2, wherein at least one instance of M is a diagnostic agent.

105. (canceled)

106. A polymer prepared by a method of claim 83.
